(19) United States
(12) Patent Application Publication
(10) Pub. No.: US 2013/0142861 A1
(43) Pub. Date: Jun. 6, 2013

(54) Compositions And Method For Detecting And Treating Abnormal Liver Homeostasis And Hepatocarcinogenesis

(71) Applicant: National Yang Ming University, Taipei (TW)

(72) Inventors: Ann-Ping Tsou, Taipei (TW); Wei-Chih Tsai, Taipei (TW); Chu-Sui Hsu, Taipei (TW)

(73) Assignee: NATIONAL YANG MING UNIVERSITY, Taipei (TW)

(21) Appl. No.: 13/689,700

(22) Filed: Nov. 29, 2012

Related U.S. Application Data
(60) Provisional application No. 61/567,038, filed on Dec. 5, 2011.

(51) Int. Cl. A61K 48/00; A61K 45/06; C12Q 1/68

(52) U.S. Cl. CPC A61K 48/005; C12Q 1/6876; A61K 45/06
USPC 424/450; 514/44 R; 506/9

(57) ABSTRACT

Novel compositions and methods for detecting and preventing and/or treating abnormal liver homeostasis and hepatocarcinoma as well as conditions that may be regulated by microRNA-122 are provided. A transgenic knockout non-human animal comprising a disruption in the endogenous mir-122 gene is also provided.

### CROSS-REFERENCE TO RELATED APPLICATION

This application claims the priority benefit of U.S. Provisional Application No. 61/567,038, filed Dec. 5, 2011, which is incorporated herein by reference in its entirety.

### GOVERNMENT SPONSORED RESEARCH

This disclosure was made with Taiwan government support under Grant Nos. 98-3112-B-010-002 and NSC99-3112-B-010-010, awarded by National Science Council and Grant No. 98A-C-T503, awarded by the Ministry of Education, Aim for the Top University Plan.

### TECHNICAL FIELD

The present disclosure relates to novel compositions and methods for detecting and preventing and/or treating abnormal liver homeostasis and hepatocarcinoma as well as conditions that may be regulated by microRNA-122. The present disclosure also relates to a transgenic knockout non-human animal comprising a disruption in the endogenous mir-122 gene.

### BACKGROUND

Hepatocellular carcinoma (HCC) is one of the most common human malignancies; this disease shows exceptional heterogeneity in cause and outcome. Despite successful local therapies such as surgery or transcatheter arterial chemoembolization, patients with HCC develop a high rate of recurrence due to local invasion and intrahepatic metastasis. Liver cancer is a complex disease involving epigenetic instability, chromosomal instability and expression abnormalities of both coding and noncoding genes; the latter includes microRNAs (miRNAs).

The capacity to fine-tune cellular gene activities via miRNAs is central to normal development, differentiation and human diseases. The strong association between miRNAs and lipid or glucose metabolism has highlighted the importance of miRNAs in the regulation of metabolic homeostasis. Many studies have supported the pivotal role of liver-specific mir-122 in lipid metabolism, HCV replication and hepatocarcinogenesis. However, mir-122's intrinsic functions remain largely undetermined.

Accordingly, there is a need to elucidate the role of mir-122, in particular in liver associated disorders or other conditions that may be regulated by mir-122.

### SUMMARY

The present disclosure provides transgenic non-human animals that comprise a disruption in the endogenous mir-122 gene. The present disclosure provides that such transgenic animals exhibit characteristics associated with liver associated disorders and is therefore useful as a model for liver associated disorders. Moreover, the present disclosure provides novel compositions and therapeutics comprising the mir-122 gene and methods of use in detecting and preventing and/or treating abnormal liver homeostasis and hepatocarcinoma as well as conditions that may be regulated by mir-122.

Accordingly, the present disclosure provides a transgenic knockout non-human animal whose genome comprises a disruption in the endogenous mir-122 gene.

In some embodiments of the present disclosure, transgenic knockout non-human animal comprises a disruption that is introduced into the genome by homologous recombination. In some embodiments of the present disclosure, the transgenic knockout non-human animal comprises a homozygous disruption of the mir-122 gene. In some embodiments of the present disclosure, the transgenic knockout non-human animal comprises a disruption that prevents the expression of a functional mir-122 RNA in the animal.

In some embodiments of the present disclosure, the transgenic knockout non-human animal comprises a global or tissue-specific disruption of the mir-122 gene. In some embodiments of the present disclosure, the transgenic knockout non-human animal comprises a germ-line deletion of the mir-122 gene. In some embodiments of the present disclosure, the transgenic knockout non-human animal comprises a tissue-specific deletion of the mir-122 gene.

In some embodiments of the present disclosure, the transgenic knockout non-human animal comprises a disruption that results from deletion of a portion of the mir-122 gene. In some embodiments of the disclosure, the transgenic knockout non-human animal comprises a disruption that results from deletion of the entire mir-122 gene.

In some embodiments of the present disclosure, the transgenic knockout non-human animal is a mouse.

In some embodiments of the present disclosure, the transgenic knockout non-human animal comprises a disruption that results in an altered phenotype compared to an animal having a wild-type mir-122 gene, wherein the altered phenotype is selected from the group consisting of hepatic steatosis, hepatitis, liver fibrosis, hepatocyte proliferation, and hepatocellular carcinoma.

The present disclosure also provides a cell or cell line isolated or derived from the transgenic knockout non-human animals whose genome comprises a disruption in the endogenous mir-122 gene.

In some embodiments of the present disclosure, the cell or cell line comprises a disruption that has been introduced into the genome by homologous recombination.

In some embodiments of the present disclosure, the cell or cell line is an undifferentiated cell selected from the group consisting of stem cell, embryonic stem cell, oocyte and embryonic cell.

The present disclosure also provides a method of generating a homozygous transgenic knockout non-human mouse whose genome comprises a disruption in the endogenous mir-122 gene, the method comprising the steps of: deleting the mir-122 gene by homologous recombination in mouse embryonic stem cells; introducing the embryonic stem cells into a mouse blastocysts and transplanting the blastocyst into a pseudopregnant mouse; allowing the blastocyst to develop into a chimeric mouse; breeding the chimeric mouse to produce offspring; and screening the offspring to identify homozygous transgenic knockout mouse whose genome comprises a deletion of the mir-122 gene.

The present disclosure also provides a method of generating a transgenic knockout non-human animal whose genome comprises a disruption in the endogenous mir-122 gene.

In some embodiments of the present disclosure, the method comprises generating the transgenic knockout non-human animal with a disruption that has been introduced into the genome by homologous recombination. In some embodiments of the present disclosure, the method comprises generating the transgenic knockout non-human animal with a disruption of the mir-122 gene that prevents the expression of a functional mir-122 RNA.

In some embodiments of the present disclosure, the method comprises generating the transgenic knockout non-human animal with a disruption that results from deletion of a portion of the mir-122 gene. In some embodiments of the present disclosure, the method comprises generating the transgenic knockout non-human animal with a disruption that results from deletion of the entire mir-122 gene.

In some embodiments of the present disclosure, the method comprises generating a transgenic knockout non-human mouse.

The present disclosure further provides a progeny of the transgenic knockout non-human animal whose genome comprises a disruption in the endogenous mir-122 gene.

In some embodiments of the present disclosure, the progeny is a mouse.

The present disclosure also provides a mir-122 knockout construct comprising a selectable marker sequence flanked by DNA sequences homologous to the mir-122 gene of a non-human animal, wherein the construct is introduced into the animal at an embryonic stage, and wherein the selectable marker sequence disrupts the mir-122 gene in the animal.

The present disclosure also provides a vector comprising the mir-122 DNA knockout construct.

The present disclosure also provides an animal model of liver associated disorders, wherein the genome of the animal model comprises a disruption in the endogenous mir-122 gene.

In some embodiments of the present disclosure, the animal model comprises a disruption that is introduced into the genome by homologous recombination. In some embodiments of the present disclosure, the animal model comprises a homozygous disruption of the mir-122 gene.

In some embodiments of the present disclosure, the animal model comprises a disruption that prevents the expression of a functional mir-122 RNA in the animal.

In some embodiments of the present disclosure, the animal model has a liver associated disorder selected from the group consisting of hepatic steatosis, hepatitis, liver fibrosis, hepatocyte proliferation, and hepatocellular carcinoma.

The present disclosure further provides a therapeutic for treating and/or preventing liver associated disorders, the therapeutic comprising a delivery vehicle carrying a mir-122 gene.

In some embodiments of the present disclosure, the therapeutic comprises a mir-122 gene that is selected from the group consisting of human mir-122 gene and murine mir-122 gene.

In some embodiments of the present disclosure, the therapeutic comprises a delivery vehicle that is a vector, a liposome, a polymer, a pharmaceutically acceptable composition, or a device which facilitates delivery of such delivery vehicle.

In some embodiments of the present disclosure, the vector is selected from the group consisting of adenovirus vectors, retrovirus vectors, adeno-associated virus vectors, herpes simplex virus vectors, SV40 vectors, polyoma virus vectors, papilloma virus vectors, picarnovirus vectors, vaccinia virus vectors, lentiviral vectors, alphaviral vectors, a helper-dependent adenovirus, and a plasmid.

In some embodiments of the present disclosure, the therapeutic is useful for treating liver associated disorders. In other embodiments of the present disclosure, the therapeutic is useful in preventing liver associated disorders. In some embodiments of the present disclosure, the liver associated disorder is selected from the group consisting of hepatic steatosis, hepatitis, liver fibrosis, hepatocyte proliferation, and hepatocellular carcinoma.

The present disclosure further provides a method of preventing and/or treating a liver associated disorder comprising administering to a subject in need thereof a therapeutically effective amount of the mir-122 gene.

In some embodiments of the present disclosure, the method relates to preventing and/or treating a liver associated disorder selected from the group consisting of hepatic steatosis, hepatitis, liver fibrosis, hepatocyte proliferation, and hepatocellular carcinoma.

In some embodiments of the present disclosure, the method comprises an administering step using a delivery vehicle. In some embodiments of the present disclosure, the delivery vehicle is a vector, a liposome, a polymer, a pharmaceutically acceptable composition, or a device which facilitates delivery of such delivery vehicle. In some embodiments of the present disclosure, the vector is selected from the group consisting of adenovirus vectors, retrovirus vectors, adeno-associated virus vectors, herpes simplex virus vectors, SV40 vectors, polyoma virus vectors, papilloma virus vectors, picarnovirus vectors, vaccinia virus vectors, lentiviral vectors, alphaviral vectors, a helper-dependent adenovirus, and a plasmid.

In some embodiments of the present disclosure, the method includes administering in a manner selected from the group consisting of intravenous administration, subcutaneous administration, intra-bone marrow administration, intra-arterial administration, intra-cardiac administration, intracerebral administration, intraspinal administration, intra-peritoneal administration, intra-muscular administration, parenteral administration, intra-rectal administration, intra-tracheal injection, intra-nasal administration, intradermal administration, epidermal administration, oral administration and combinations thereof.

In some embodiments of the present disclosure, the method includes administering to the mammal in need of treatment multiple therapeutically effective amounts of the mir-122 gene.

In some embodiments of the present disclosure, the method includes administering the mir-122 gene in combination with another therapeutic, such as other anticancer therapeutics or therapies.

In some embodiments of the present disclosure, the subject is a human.

The present disclosure also provides a method for detecting the presence or a predisposition to a liver associated disorder in a subject, comprising the steps of: obtaining a test sample from the subject; determining the level of mir-122 expression in the test sample; comparing the mir-122 expression level from the test sample to the expression level present in a control sample known not to have, or not to be predisposed to a liver associated disorder, wherein an alteration in the level of mir-122 expression in the test sample as compared to the control sample indicates the presence or predisposition to a liver associated disorder.

In some embodiments of the present disclosure, the liver associated disorder is selected from the group consisting of hepatic steatosis, hepatitis, liver fibrosis, hepatocyte proliferation, and hepatocellular carcinoma.

In some embodiments of the present disclosure, the method for detecting the presence or a predisposition to a liver associated disorder in a subject involves detecting a decreased level of mir-122 expression in the test sample as compared to the control sample.

The present disclosure also provides a method for screening a candidate agent for the ability to treat and/or prevent liver associated disorder comprising: providing a transgenic knock-out non-human animal whose genome comprises a disruption in the endogenous mir-122 gene, wherein the animal exhibits an altered phenotype selected from the group consisting of hepatic steatosis, hepatitis, liver fibrosis, hepatocyte proliferation, and hepatocellular carcinoma; administering to the animal the candidate agent, and evaluating the animal to determine whether the candidate agent affects and/or ameliorates at least one of the altered phenotypes.

In some embodiments of the present disclosure, the candidate agent is a mir-122 target gene. In some embodiments of the present disclosure, the target gene is selected from the group consisting of AlpI, Cs, Ctgf, Igf2, Jun, Klf6, Prom1 and Sox4.

These and other features, aspects and advantages of the present disclosure will become better understood with reference the following description, examples and appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Generation of mir-122 deletion mice. a. Strategy to generate mir-122 deletion mice by homologous recombination. The BAC clone bMQ-418A13 (chr18:65269984-65437465) containing the entire mmu-mir-122 locus was purchased from Geneservice (Cambridge, UK). A genomic fragment of 13 kb encompassing 7.8 kb upstream and 5.1 kb downstream of pre-mir-122 was cloned to PL253 in bacteria strain EL350 by recombineering-based method (Liu, P. et al., Genome Res 13, 476-84 (2003)). The genomic fragment of mir-122 constructed in PL253 was used to replace the wild-type allele of mir-122 in 129Sv mouse embryonic stem cells (MESC). MESC clones containing the targeted allele were identified by Southern blot analysis. Several clones were isolated and transfected with a vector encoding the Cre recombinase to delete a fragment of 1544 bp containing the entire pre-mir-122. Clones with the mir-122 knockout allele were identified by Southern blot analysis and were injected into C57BL/6J blastocysts. Germline transmission of the mir-122^{-/-} allele was achieved by crossing the chimeric mice with normal C57BL/6 mice. The homozygous mir-122^{-/-} mice were generated with littermates from the intercross of the heterozygous mice. b. Genotyping of F1 and successive progenies was performed by Southern blotting with Scal digested DNA. WT, 9667 bp; homozygous deletion of mir-122, 8123 bp. c. Genotyping with genomic PCR. WT, 429 bp; mir-122^{-/-}, 825 bp.

FIG. 2. Pathophysiological features of mir-122^{-/-} mice. a. Total serum cholesterol, fasting triglyceride (TG), alkaline phosphatase (ALP) and alanine aminotransferase (ALT) were measured enzymatically on a DRI-CHEM3500S autoanalyzer (FUJIFILM). n=20 mice per group. [symbol] , mir-122^{+/+}; [symbol] , mir-122^{-/-}. b. mir-122^{-/-} livers exhibited progressive accumulation of lipid (Oil Red O) and reduced glycogen storage (Periodic acid—Schiff, PAS). n=6. c. Progressive increase in the numbers of Kupffer cells in mir-122^{-/-} livers (F4/80 antibody). Activation of hepatic stellate cells near the portal regions (Sirius Red and anti-desmin antibody). Bar on the histological sections, 100 μm. n=6. d. The number of Kupffer cells (anti-F4/80) per high-power field was counted. n=10 microscopic fields at 200×. e. Quantitative real-time polymerase chain reaction (qRT-PCR) for two markers of fibrosis (Tgfb1 and Ctgf). n=5 for Tgfb1 and n=8 for Ctgf to normalize the individual variation. f. Western blot analysis of Desmin expression. Data shown are representative of five independent experiments. *p<0.05, **p<0.01.

FIG. 3. Liver damage in mir-122^{-/-} mice is reversible. a. Serum levels of lipoproteins (Hydragel K20). C: normal human serum; WT: miR-122^{+/+} (1, 2, 3); KO: mir-122^{-/-} (4, 5, 6). b. Western blot analysis of the serum apoproteins, apoB-100, apoB-48 and apoE. c. qRT-PCR analysis of the genes involved in lipogenesis, bile metabolism, VLDL export and transcription regulation. All values were normalized relative to the level of β₂-microglobulin mRNA. Gene expression as fold change was plotted relative to the level of WT mice. n=5. d. Western blot analysis of hepatic proteins. Fasn, fatty acid synthase; Mttp, microsomal triglyceride transfer protein; apoE. Data shown are representative of three independent experiments. d. ¹H NMR spectra of hepatic lipid contents. All values were represented as mg/g liver tissue. f-i. Twenty microgram of endotoxin-free pCMV6-Neo was delivered into the tail vein of WT mice (WT-pCMV6, [symbol] ), pCMV6-Neo to mir-122^{-/-} mice (KO-pCMV6, ▪) and pCMV6-Mttp to mir-122^{-/-} mice (KO-Mttp, [symbol] ) by the hydrodynamic injection protocol. f. Serum indexes for cholesterol and TG, serum levels of lipoproteins analyzed at one-month. Each group included five mice of 3-month old. g. Restoration of Mttp at one-month resulted in a drastic reduction in fatty accumulation (Oil Red O), in inflammation (F4/80 IHC, i) and in collagen deposition (Sirius Red). j-m. Twenty microgram of pcDNA3.1-HA (HA) was delivered into the tail vein of WT mice (HA, [symbol] ), pcDNA3.1-HA to mir-122^{-/-} mice (HA, ▪) and pcDNA3.1-HA-miR-122 to mir 122^{-/-} mice (122, [symbol] ) by hydrodynamic injection. j. Serum indexes for cholesterol, TG, ALP and ALT analyzed at 14-days. n=5. While blood ALT level was reduced in mice received miR-122 construct, the differences did not reach statistical significance due to high variability among the individuals. k. Restoration of mir-122 at one-month resulted in a drastic reduction in fatty accumulation (Oil Red O) and in collagen accumulation and activation of stellate cell (Sirius Red and anti-Desmin). Moderate increase in glycogen storage was noticed (PAS). Bar on the histological sections, 100 μm. n=5. h, l. qRT-PCR assay of lipid metabolic genes (Acyl, Fasn, Pklr, and Mttp). n=3. m. qRT-PCR assay of markers of fibrosis (Ctgf, Klf6 and Tgfb1). n=3 for Tgfb1 and Klf6. n=6 for Ctgf to normalize the individual variation. Asterisks indicate significant differences for vehicle control-injected miR-122^{-/-} mice (*p<0.05, **p<0.01, ***p<0.001; Student's t test) relative to vehicle control-injected WT mice. # indicate significant differences (## p<0.01, ### p<0.001; Student's t test) of gene-restored miR-122^{-/-} mice relative to vehicle control-injected miR-122^{-/-} mice.

FIG. 4. 1H NMR spectra of lipid extracts from liver of (A) wild type (WT) and (B) mir-122−/− mice (mir-122K0). Identified peak: 1. total cholesterol C-18, CH₃; 2. total cholesterol C-26, CH₃/C-27, CH₃; 3. Fatty acyl chain CH₃(CH₂)ₙ; 4. Total cholesterol C-21, CH₃; 5. free cholesterol C-19, CH₃; 6. Esterified cholesterol C-19, CH₃; 7. multiple cholesterol protons; 8. fatty acyl chain (CH₂)ₙ; 9. Multiple cholesterol protons; 10. fatty acyl chain —CH₂CH₂CO; 11. multiple cholesterol protons; 12. Fatty acyl chain —CH₂CH═; 13. fatty acyl chain —CH₂CO; 14. fatty acyl chain ═CHCH₂CH═; 15. Sphingomyelin and choline N(CH₃)₃; 16. free cholesterol C-3, CH; 17. phosphatidylcholine N—CH₂; 18. glycerophospholipid backbone C-3, CH₂; 19. glycerol backbone C-1, CH₂; 20. glycerol backbone C-3, CH₂; 21. phosphatidylcholine PO—CH₂; 22. esterified cholesterol C-3, CH; 23. Glycerolphospholipid backbone C-2, CH; 24. fatty acyl chain —HC═CH—.

FIG. 5. Restoration of mir-122 in mir-122^{-/-} mice by hydrodynamic injection. Twenty μg plasmid DNA of endotoxin-free pcDNA3.1-HA (HA) or pcDNA3.1-HA-miR-122 (122) was delivered in tail vein of WT (HA, [symbol] ) or mir-122^{-/-} (122, [symbol] ) mice by hydrodynamic injection. a. Genotyping with genomic PCR. WT, 429 bp; mir-122^{-/-}, 825 bp. b. Expression of mir-122 in mir-122^{-/-} livers was detected a month after hydrodynamic injection by qRT-PCR.

FIG. 6. Loss of mir-122 leads to abnormal glucose metabolism. a. mir-122^{-/-} (KO) livers exhibit low level of glycogen storage shown by PAS staining. P78, postnatal 78 days; P180, postnatal 180 days. b. Both phosphorylation and the protein level of hepatic glycogen synthase (Gys2) were reduced in mir-122^{-/-} livers. c. mir-122^{-/-} mice had slightly higher glucose levels as shown in the glucose tolerance test. *P<0.05, **P<0.01.

FIG. 7. Mir-122^{-/-} mice develop liver tumors. a. Summary of the tumor incidence in male (left) and in female (right) mir-122^{-/-} mice. b. Liver lesions and liver tumors in male mice of 11-month and 14-month old, respectively. The representative liver of mir-122^{-/-} mouse at 11-month reveals a small round-shaped solid tumor (yellow arrow, approximately 3 mm in diameter). Three representative livers of 14-month mir-122^{-/-} mice show multiple larger tumors with sizes ranges from 6 mm to 12 mm in diameters. n=6. Bar on the histological sections, 3 mm (Front), 2 mm (HE, 0.5×) and 100 μm (HE, 10×; anti-Pcna). Inset: a representative 400× magnification field. The dotted lines show the edges of normal liver area (N) and tumor area (T). Note that the tumors have invasive edges. c. qRT-PCR assay of onco-fetal genes (Afp, Igf2, Src) and tumor-initiating cell markers (Prom1, Thy1 and Epcam). [symbol] , WT; ▪, tumor adjacent normal tissues; [symbol] , tumor. n=3. d. Expression of E-cadherin is down-regulated and that of vimentin is up-regulated in mir-122^{-/-} tumor tissues. Periportal distribution of E-cadherin protein in normal liver. n=5. Bar on the histological sections, 100 μm. e. Left, qRT-PCR assay of Cdh1 and Vim. [symbol] , WT; ▪, tumor adjacent normal tissues; [symbol] , tumor. n=3. *p<0.05, **p<0.01, ***p<0.001; Student's t test relative to WT mice. Right, Western blot analysis of E-cadherin and vimentin. Gapdh is the loading control. WT: normal liver; T: tumors. Data shown are representative of four independent experiments. f. The livers of 14-months old mice were isolated and examined by immunoblot analysis to detect Pten, p-Akt, Akt, p-craf, c-raf, p-Mek1/2, Mek1/2, p-Erk and Erk. Gapdh is the loading control. WT: normal liver; N: tumor adjacent normal tissues; T: tumors. Data shown are representative of three independent experiments. g. Long-term restoration of mir-122 resulted in a drastic reduction in tumor incidence and tumor sizes of mir-122^{-/-} mice. Twenty microgram of pcDNA3.1-HA (HA) was delivered into the tail vein of WT mice (WT-HA), pcDNA3.1-HA to mir-122^{-/-} mice (KO-HA) and pcDNA3.1-HA-miR-122 to mir-122^{-/-} mice (KO-122) by hydrodynamic injection for a period of 8 months. A small tumor depicted in KO-122 mouse. The dotted lines show the edges of normal liver area (N) and tumor area (T). Note that the tumor in KO-122 mouse has smooth edge while tumors of KO-HA mouse have invasive edges. h. Summary of the tumor incidence.

FIG. 8. Development of HCC in female mir-122^{-/-} mice. a. Serum profile of female mir-122^{-/-} mice. Total serum cholesterol, fasting triglyceride (TG), alkaline phosphatase (ALP) and alanine aminotransferase (ALT) were measured enzymatically on a DRI-CHEM3500S autoanalyzer (FUJIFILM). n=20 mice per group. Female mir-122 KO mice exhibited similar serum profiles (low cholesterol/triglyceride and high ALP/ALT) as found in the male mir-122 KO mice (FIG. 2a). b. Female mir-122^{-/-} mice developed hepatic fibrosis (Sirius Red), inflammation (F4/89 for Kupffer cells) and accumulated less glycogen (PAS staining) as seen in the male mutant mice (FIG. 2b). c. Summary of tumor incidence. d. Serum level of estrasdiol was measured by RIA. n=20 mice per group. There was a slight but not significant reduction in the serum estradiol in the older mice. e. Serum levels of 116 were measured by ELISA. n=5 mice per group. Serum 116 was not detected in the normal female mice. Significant increase of serum 116 was detected in older female miR-122 KO mice. [symbol] , mir-122^{+/+}; [symbol] , mir-122⁻⁻.

FIG. 9. Blood vessel distributions of the tumors as revealed by immunohistochemistry staining using the Cd31 antibody. Twenty micrograms of pcDNA3.1-HA (HA) were delivered into the tail veins of WT mice (122^{+/+}-HA, [symbol] ), pcDNA3.1-HA to the mir-122^{-/-} mice (122^{-/-}-HA, ▪) and pcDNA3.1-HA-miR-122 to the mir-122^{-/-} mice (122^{-/-}-122, [symbol] ) by hydrodynamic injection for a period of 8 months. Left, Immunohistochemistry. The dotted lines show the edges of the normal liver area (N) and tumor area (T). Bar on the histological sections, 100 μm. Right, Bar chart shows comparisons of the mean Cd31 positive vessel numbers per high power field of the various tissue sections. Significant reductions in the number of Cd31 positive blood vessels were found for the tumors of the 122-restored mutant mice groups ( [symbol] ) compared to the tumors of the control HA-plasmid injected mutant mice group (▪). Microvessels that stained positively with the anti-Cd31 antibody were counted in 10 different microscopic fields at 200× using the Aperio Positive Pixel Count v9 software. Due to the small size of the mass, only 2 to 4 fields were counted for the 122^{-/-}-122 tumors. The mean value of the fields was calculated to provide a mean microvessel density. n=3 for 122^{+/+}-HA; n=2 for 122^{-/-}-HA and 122^{-/-}-122.

FIG. 10. Mir-122 deletion changes the global gene expression and the novel target genes contributing to liver fibrosis can be identified. a. GSEA (Gene set enrichment analysis) of liver tissues from 2-month-old mice and tumor tissues from 11-month- and 14-month-old male mir-122^{-/-} mice. Notable gene sets are displayed with normalized enrichment score for each comparison. NES, normalized enrichment score with positive and negative scores indicating enrichment and de-enrichment in mir-122^{-/-}, respectively. FDR, false detection rate. p, nominal p value. * p-value<0.05 or FDR q-value<0.25, ** p-value<0.05 and FDR q-value<0.25. b. Heat map of 91 genes in the KEGG “pathways in cancer” differentially expressed in livers of 2-month-old mice and tumor tissues from 11-month- and 14-month-old male mir-122^{-/-} mice (cutoff 1.5). The heat scale on the side of the map represents changes on a linear scale. Red and blue colors denote up-regulated and down-regulated expressions, respectively. Relative expression levels of genes in KEGG “Pathway in cancer” gene set are listed in Supplementary Table 4. c. A 3′UTR reporter assay was used to verify novel targets that were predicted (Supplementary Table 5). Eight 3′UTR constructs demonstrated a significant reduction in luciferase activity in HEK293T cells overexpressing miR-122 (293T-122). 3′UTR constructs of Aldoa and B2m are the positive and the negative controls, respectively. d. Expression of Klf6 is increased at both the mRNA (left) and protein level (right) in mir-122^{-/-} livers. e. Diagram depicting the 3′UTR reporter assays with two binding site mutations (mu1 and mu2) within the 3′UTR of the Klf6 transcript. f. Reduction of luciferase activity driven by Klf6-3′UTR construct was observed in cells expressing wild type miR-122 (293T-122) but not in cells overexpressing mutant miR-122 (293T-122M). 293T-GFP acts as the control in 293T cells. g. Reporter constructs containing the single miR-122 binding site mutation (Klf6-mu1 or -mu2) suppress luciferase activity less efficiently compared to Klf6-WT. The construct containing double mutations (Klf6-mu1+mu2) failed to suppress luciferase activity.

FIG. 11. Mir-122 deletion changes the global gene expression. a. Heat map of the 886 genes that were differentially expressed in the livers of 2-month-old male mir-122^{-/-} and WT mice (cutoff 1.5). The heat scale at the bottom of the map represents changes on a linear scale. Red and blue colors denote up-regulated and down-regulated expressions, respectively. b. GSEA (Gene set enrichment analysis). Enrichment plots of the top three pathways significantly de-enriched in the mir-122^{-/-} mice. Enrichment plots of the significantly up-regulated pathways in the mir-122^{-/-} mice, cell communication (Focal adhesion, Gap junction, Tight junction), cell-cell interaction (Cell adhesion molecules, ECM-receptor interaction), fibrogenic pathways (Liver fibrosis and TGF-beta signaling), signal transduction (MAPK signaling) and major cancer-related phenotypes. NES, normalized enrichment score with the positive and negative scores indicating enrichment and de-enrichment in mir-122^{-/-}, respectively. FDR, false detection rate. p, nominal p value. The complete results of the GSEA analysis are listed in Supplementary Table 2.

FIG. 12. An enlarged version of FIG. 10b that shows the gene symbols of the differentially expressed genes. The relative expression levels of the genes in the KEGG “Pathway in cancer” gene set are described in Supplementary Table 4. Expression patterns of genes in the KEGG “pathways in cancer” display age-dependent change patterns in the tissues from the mir-122^{-/-} mice.

FIG. 13. siRNA-mediated knockdown of Ctgf and Klf6 led to a decrease in hepatic fibrogenesis in the mir-122^{-/-} mice. The hydrodynamic injection of shCtgf reduced the expression of Ctgf as shown by western blotting (a) and IHC (b). The hydrodynamic injection of shKlf6 reduced the expression of Klf6 as shown by western blotting (c). Reduced collagen deposition (Sirius Red staining) was seen in the mir-122^{-/-} mice that received either shCtgf (b) or shKlf6 (d) but not in mice that received shLuc, which was the control shRNA against the Luciferase gene. n=3 mice per group. mir-122^{+/+}: wild-type mice; mir-122^{-/-}: mir-122 KO mice.

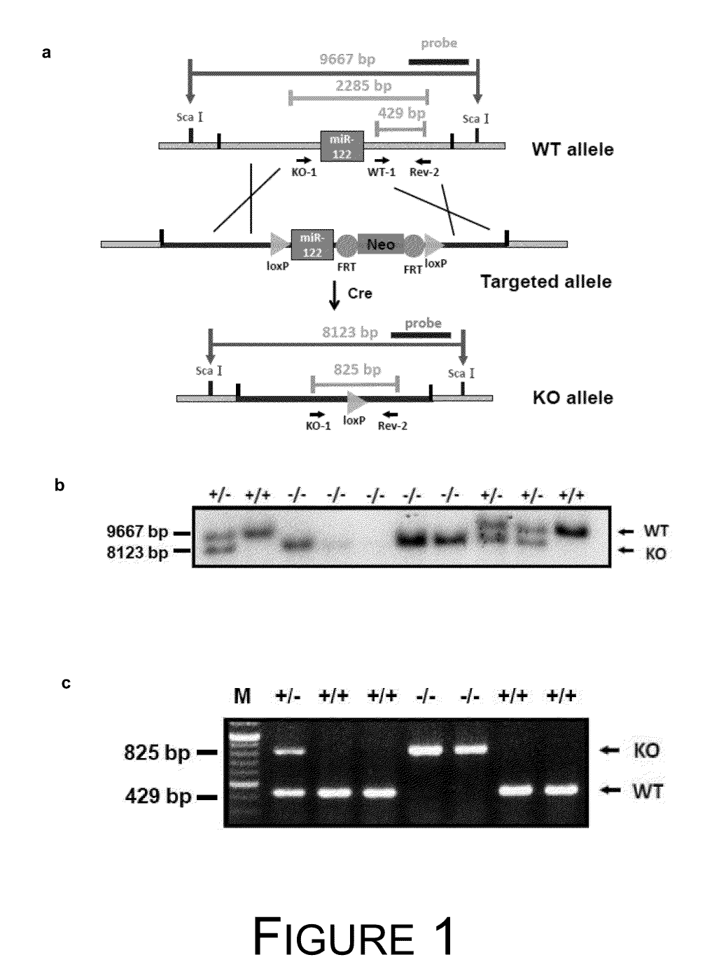

### DETAILED DESCRIPTION OF PREFERRED EMBODIMENTS

Unless otherwise defined, all technical terms used herein have the same meaning as commonly understood by one of ordinary skill in the art to which the present disclosure belongs.

As used herein, the terms “treating” and “treatment” are used to refer to administration or application of a therapeutic agent to a subject or performance of a procedure or modality on a subject for the purpose of obtaining a therapeutic benefit of a disease or health-related condition.

As used herein, the terms “preventing,” “inhibiting,” “reducing” or any variation of these terms, includes any measurable decrease or complete inhibition to achieve a desired result. For example, there may be a decrease of 5%, 10%, 15%, 20%, 25%, 30%, 35%, 40%, 45%, 50%, 55%, 60%, 65%, 70%, 75%, 80%, 85%, 90%, 95%, 99% or more, or any range derivable therein, reduction of activity or symptoms, compared to normal.

As used herein, the terms “administered” and “delivered” are used to describe the process by which a composition of the present disclosure is administered or delivered to a subject, a target cell or are placed in direct juxtaposition with the target cell. The terms “administered” and “delivered” are used interchangeably.

As used herein, the terms “patient,” “subject” and “individual” are used interchangeably herein, and mean a mammalian (e.g., human) subject to be treated and/or to obtain a biological sample from.

As used herein, the term “effective” means adequate to accomplish a desired, expected, or intended result. For example, an “effective amount” may be an amount of a compound sufficient to produce a therapeutic benefit.

As used herein, the terms “therapeutically effective” or “therapeutically beneficial” refers to anything that promotes or enhances the well-being of the subject with respect to the medical treatment of a condition. This includes, but is not limited to, a reduction in the onset, frequency, duration, or severity of the signs or symptoms of a disease.

As used herein, the term “therapeutically effective amount” is meant an amount of a composition as described herein effective to yield the desired therapeutic response.

As used herein, the terms “diagnostic,” “diagnose” and “diagnosed” mean identifying the presence or nature of a pathologic condition.

As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used as described herein.

The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

As used herein, the term “sample” is used herein in its broadest sense. For example, a sample including polynucleotides, peptides, antibodies and the like may include a bodily fluid, a soluble fraction of a cell preparation or media in which cells were grown, genomic DNA, RNA or cDNA, a cell, a tissue, skin, hair and the like. Examples of samples include biopsy specimens, serum, blood, urine, plasma and saliva.

Although methods and compositions similar or equivalent to those described herein can be used in the practice or testing of the present disclosure, suitable methods and compositions are described below.

As used herein, the term “treatment” is defined as the application or administration of a therapeutic agent to a patient, or application or administration of the therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease, or the predisposition toward disease. For example, “treatment” of a patient in whom no symptoms or clinically relevant manifestations of a disease or disorder have been identified is preventive or prophylactic therapy, whereas clinical, curative, or palliative “treatment” of a patient in whom symptoms or clinically relevant manifestations of a disease or disorder have been identified generally does not constitute preventive or prophylactic therapy.

It is specifically contemplated that any limitation discussed with respect to one embodiment of the present disclosure may apply to any other embodiment of the present disclosure. Furthermore, any composition of the present disclosure may be used in any method of the present disclosure, and any method of the present disclosure may be used to produce or to utilize any composition of the present disclosure.

The particular embodiments discussed below are illustrative only and not intended to be limiting.

### Mir-122 Transgenic Knockout Animals and Cells

Mir-122 is a liver-specific miRNA that is well conserved within vertebrates. Mir-122 has been implicated in the regulation of lipid metabolism, HCV replication and hepatocarcinogenesis. The sequence of mir-122 is well conserved between different mammalian species.

The present disclosure provides for the first time a novel method to study the mechanism of mir-122 regulation in livers using an in vivo loss-of-function model. The present disclosure provides that mir-122 modulates the expression of multiple genes involved in hepatocyte differentiation and proliferation. The present disclosure provides that mice lacking mir-122 (mir-122^{-/-}) are viable but develop temporally controlled staging of the disease with an early onset of steatohepatitis and fibrosis, followed by late occurring liver lesions and HCC. A striking gender disparity in HCC with a male-to-female ratio of 3.9:1 recapitulates the disease incidence in humans. The loss of mir-122 expression seems to enable the reprogramming of hepatocyte differentiation and quiescence. Thus, these mice are useful as a model of the human disease. Furthermore, detection of the levels of activity or expression of mir-122 is useful for the presence as well as early diagnosis and prognosis of liver associated disorders.

The present disclosure also provides that an impairment in Mttp and VLDL assembly led to steatosis, which can be corrected with in vivo restoration of Mttp expression. Thus, the present disclosure provides a disease model in which liver disorders arise via the functional coordination of various direct and indirect genes of mir-122, with Mttp being one such essential gene that is essential for the mir-122 null phenotype of steatosis and is likely regulated by mir-122 target gene(s) in a network-like fashion.

The present disclosure further provides that re-expression of mir-122 leads to significant reduction in the incidence of steatohepatitis, fibrosis and HCC. Moreover, hepatic fibrosis in mir-122^{-/-} mice is partially attributed to the actions of two mir-122 targets, Klf6 and Ctgf. These results support a role for mir-122 as a crucial regulator of hepatic homeostasis and indicate that in vivo miR-122 restoration may contribute to metabolic normalization and tumor regression in HCC and may have potential application for anti-cancer treatment of miR-122-low HCC.

Accordingly, the present disclosure describes for the first time a transgenic knockout non-human animal and cell or cell lines derived therefrom whose genome comprises a disruption in the endogenous mir-122 gene. In some embodiments, the transgenic knockout non-human animal comprises a disruption that is introduced into the genome by homologous recombination. In other embodiments, the transgenic knockout non-human animal comprises a disruption that is a homozygous disruption of the mir-122 gene. In other embodiments, the transgenic knockout non-human animal comprises a disruption that prevents the expression of a functional mir-122 RNA in the animal. In still other embodiments, the transgenic knockout non-human animal comprises a global or tissue-specific disruption of the mir-122 gene. The transgenic knockout non-human animal may comprise a germ-line deletion of the mir-122 gene. Alternatively, the transgenic knockout non-human animal may comprise a tissue-specific deletion of the mir-122 gene.

In other embodiments, the transgenic knockout non-human animal of the present disclosure may comprise a disruption that results from deletion of a portion of the mir-122 gene. In other embodiments, the transgenic knockout non-human animal may comprise a disruption that results from deletion of the entire mir-122 gene.

In some embodiments, the transgenic animal of the present disclosure can be any non-human mammal, preferably a mouse. A transgenic animal can also be, for example, any other non-human mammals, such as rat, rabbit, goat, pig, dog, cow, or a non-human primate. It is understood that transgenic animals that have a disruption in the mir-122 gene or other mutated forms that decrease the expression of mir-122, can be used in the methods of the present disclosure.

In some embodiments, the transgenic knockout non-human animal of the present disclosure comprises a disruption that results in an altered phenotype compared to an animal having a wild-type mir-122 gene, wherein the altered phenotype is selected from the group consisting of hepatic steatosis, hepatitis, liver fibrosis, hepatocyte proliferation, and hepatocellular carcinoma.

The present disclosure also provides for a method of generating a homozygous transgenic knockout non-human mouse whose genome comprises a disruption in the endogenous mir-122 gene comprising the steps of: deleting the mir-122 gene by homologous recombination in mouse embryonic stem cells; introducing the embryonic stem cells into a mouse blastocysts and transplanting the blastocyst into a pseudopregnant mouse; allowing the blastocyst to develop into a chimeric mouse; breeding the chimeric mouse to produce offspring; and screening the offspring to identify homozygous transgenic knockout mouse whose genome comprises a deletion of the mir-122 gene.

The present disclosure further provides for a method of generating a transgenic knockout non-human animal described herein. In some embodiments, the disruption has been introduced into the genome by homologous recombination. In other embodiments, the disruption prevents the expression of a functional mir-122 RNA. In other embodiments, the disruption results from deletion of a portion of the mir-122 gene. In some embodiments, the disruption results from deletion of the entire mir-122 gene.

The present disclosure also provides for a cell or cell line isolated or derived from the transgenic knockout non-human animal described herein. In some embodiments, the cell or cell line comprises a disruption that has been introduced into the genome by homologous recombination. In some embodiments, the cell or cell line comprises a disruption that prevents the expression of a functional mir-122 RNA. In some embodiments, the cell or cell line comprises a disruption that results from deletion of a portion of the mir-122 gene. In some embodiments, the cell or cell line comprises a disruption that results from deletion of the entire mir-122 gene. In some embodiments, the cell or cell line is derived from a transgenic knockout mouse. In some embodiments, the cell or cell line is an undifferentiated cell selected from the group consisting of stem cell, embryonic stem cell, oocyte and embryonic cell.

The present disclosure further provides for a progeny of the transgenic knockout non-human animal described herein. In some embodiments, the progeny is can be any non-human mammal, preferably a mouse. The progeny of the transgenic knockout non-human animal can also be, for example, any other non-human mammals, such as rat, rabbit, goat, pig, dog, cow, or a non-human primate.

The present disclosure also provides for a mir-122 knockout construct comprising a selectable marker sequence flanked by DNA sequences homologous to the mir-122 gene of a non-human animal, wherein the construct is introduced into the animal at an embryonic stage, the selectable marker sequence disrupts the mir-122 gene in the animal. The present disclosure also provides a vector comprising a mir-122 DNA knockout construct.

The animals, cells, and methods of the present disclosure are performed using mir-122^{-/-} cells and animals. mir-122^{-/-} animals and cells are generated as described herein, typically by targeting a genomic copy of the mir-122 gene for disruption and ultimately by eliminating or greatly decreasing mir-122 function in an animal or cell. Preferably, such targeted disruption will occur in the liver of the animal. In a more preferred embodiment, mir-122 gene disruption will occur almost exclusively or exclusively in liver tissue.

The targeting construct of the present disclosure may be produced using standard methods known in the art. For example, the targeting construct may be prepared in accordance with conventional ways, where sequences may be synthesized, isolated from natural sources, manipulated, cloned, ligated, subjected to in vitro mutagenesis, primer repair, or the like. At various stages, the joined sequences may be cloned, and analyzed by restriction analysis, sequencing, or the like.

The targeting DNA can be constructed using techniques well known in the art. For example, the targeting DNA may be produced by chemical synthesis of oligonucleotides, nick-translation of a double-stranded DNA template, polymerase chain reaction amplification of a sequence (or ligase chain reaction amplification), purification of prokaryotic or target cloning vectors harboring a sequence of interest (e.g., a cloned cDNA or genomic DNA, synthetic DNA or from any of the aforementioned combination) such as plasmids, phagemids, YACs, cosmids, bacteriophage DNA, other viral DNA or replication intermediates, or purified restriction fragments thereof, as well as other sources of single and double-stranded polynucleotides having a desired nucleotide sequence. Moreover, the length of homology may be selected using known methods in the art. For example, selection may be based on the sequence composition and complexity of the predetermined endogenous target DNA sequence(s).

The targeting construct of the present disclosure typically comprises a first sequence homologous to a portion or region of the mir-122 gene and a second sequence homologous to a second portion or region of the mir-122 gene. The targeting construct further comprises a positive selection marker, which is preferably positioned in between the first and the second DNA sequence that are homologous to a portion or region of the target DNA sequence. The positive selection marker may be operatively linked to a promoter and a polyadenylation signal.

Other regulatory sequences known in the art may be incorporated into the targeting construct to disrupt or control expression of a particular gene in a specific cell type. In addition, the targeting construct may also include a sequence coding for a screening marker, for example, green fluorescent protein (GFP), or another modified fluorescent protein.

Although the size of the homologous sequence is not critical and can range from as few as 50 base pairs to as many as 100 kb, preferably each fragment is greater than about 1 kb in length, more preferably between about 1 and about 10 kb, and even more preferably between about 1 and about 5 kb. One of skill in the art will recognize that although larger fragments may increase the number of homologous recombination events in ES cells, larger fragments will also be more difficult to clone.

Generally, a sequence of interest is identified and isolated from a plasmid library in a single step using, for example, long-range PCR. Following isolation of this sequence, a second polynucleotide that will disrupt the target sequence can be readily inserted between two regions encoding the sequence of interest. In accordance with this aspect, the construct is generated in two steps by (1) amplifying (for example, using long-range PCR) sequences homologous to the target sequence, and (2) inserting another polynucleotide (for example a selectable marker) into the PCR product so that it is flanked by the homologous sequences. Typically, the vector is a plasmid from a plasmid genomic library. The completed construct is also typically a circular plasmid.

In another embodiment, the targeting construct may contain more than one selectable maker gene, including a negative selectable marker, such as the herpes simplex virus tk (HSV-tk) gene. The negative selectable marker may be operatively linked to a promoter and a polyadenylation signal.

Once an appropriate targeting construct has been prepared, the targeting construct may be introduced into an appropriate host cell using any method known in the art. Various techniques may be employed in the present disclosure, including, for example, pronuclear microinjection; retrovirus mediated gene transfer into germ lines; gene targeting in embryonic stem cells; electroporation of embryos; sperm-mediated gene transfer; and calcium phosphate/DNA co-precipitates, microinjection of DNA into the nucleus, bacterial protoplast fusion with intact cells, transfection, polycations, e.g., polybrene, polyomithine, etc., or the like. Various techniques for transforming mammalian cells are known in the art.

Any cell type capable of homologous recombination may be used in the practice of the present disclosure. Examples of such target cells include cells derived from vertebrates including mammals such as, murine species, bovine species, ovine species, simian species, and other eukaryotic organisms.

Preferred cell types include embryonic stem (ES) cells, which are typically obtained from pre-implantation embryos cultured in vitro. The ES cells are cultured and prepared for introduction of the targeting construct using methods well known to the skilled artisan. The ES cells that will be inserted with the targeting construct are derived from an embryo or blastocyst of the same species as the developing embryo into which they are to be introduced. ES cells are typically selected for their ability to integrate into the inner cell mass and contribute to the germ line of an individual when introduced into the mammal in an embryo at the blastocyst stage of development. Thus, any ES cell line having this capability is suitable for use in the practice of the present disclosure.

After the targeting construct has been introduced into cells, the cells where successful gene targeting has occurred are identified. Insertion of the targeting construct into the targeted gene is typically detected by identifying cells for expression of the marker gene. In a preferred embodiment, the cells transformed with the targeting construct of the present disclosure are subjected to treatment with an appropriate agent that selects against cells not expressing the selectable marker. Only those cells expressing the selectable marker gene survive and/or grow under certain conditions. For example, cells that express the introduced neomycin resistance gene are resistant to the compound G418, while cells that do not express the neo gene marker are killed by G418. If the targeting construct also comprises a screening marker such as GFP, homologous recombination can be identified through screening cell colonies under a fluorescent light. Cells that have undergone homologous recombination will have deleted the GFP gene and will not fluoresce.

Successful recombination may be identified by analyzing the DNA of the selected cells to confirm homologous recombination. Various techniques known in the art, such as PCR and/or Southern analysis may be used to confirm homologous recombination events.

Selected cells are then injected into a blastocyst (or other stage of development suitable for the purposes of creating a viable animal, such as, for example, a morula) of an animal (e.g., a mouse) to form chimeras. Alternatively, selected ES cells can be allowed to aggregate with dissociated mouse embryo cells to form the aggregation chimera. A chimeric embryo can then be implanted into a suitable pseudopregnant female foster animal and the embryo brought to term. Chimeric progeny harboring the homologously recombined DNA in their germ cells can be used to breed animals in which all cells of the animal contain the homologously recombined DNA. In one embodiment, chimeric progeny mice are used to generate a mouse with a heterozygous disruption in the mir-122 gene. Heterozygous transgenic mice can then be mated. It is well known in the art that typically ¼ of the offspring of such matings will have a homozygous disruption in the mir-122 gene.

The heterozygous and homozygous transgenic mice can then be compared to normal, wild type mice to determine whether disruption of the mir-122 gene causes phenotypic changes, especially pathological changes. For example, heterozygous and homozygous mice may be evaluated for phenotypic changes by physical examination, necropsy, histology, clinical chemistry, complete blood count, body weight, organ weights, and cytological evaluation of various tissues, e.g., liver tissue.

### Animal Models of Liver Associated Disorders

The present disclosure provides models for analysis of liver associated disorders in a non-human mammal, e.g., a mouse. In some embodiments, the animal model comprises a genome with a disruption in the endogenous mir-122 gene. In some embodiments, the animal model comprises a disruption that is introduced into the genome by homologous recombination. In some embodiments, the animal model comprises a homozygous disruption of the mir-122 gene. In some embodiments, the animal model comprises a disruption that prevents the expression of a functional mir-122 RNA.

Homozygous disruption of the mouse mir-122 gene results in the development of temporally controlled staging of disease with early onset of hepatic steatosis and fibrosis, followed by late occurring liver lesions and HCC. This disease progression closely follows liver cancer progression in humans. Animals comprising a homozygous disruption of the mouse mir-122 gene can be used to analyze liver cancer progression. In addition, cancerous cells can be obtained from the mir-122^{-/-} animals and used for analysis of the molecular basis of the disease.

Accordingly, in some embodiments, the animal model has a liver associated disorder selected from the group consisting of hepatic steatosis, hepatitis, liver fibrosis, hepatocyte proliferation, and hepatocellular carcinoma.

Because of the similarity in progression between human liver associated cancer development and the liver associated cancer related to mir-122 disruption, murine mir-122-related liver associated cancer can be used to identify compounds and treatments that have a therapeutic effect on human liver cancer. Compounds or treatments can be tested on whole animals, i.e., mice, that have a mir-122 disruption or can be tested on cells or cell lines derived from animals that have a mir-122 disruption.

### Therapeutic Uses

The present disclosure describes that mir-122 restoration was able to lead to metabolic normalization and tumor regression, as evidenced by the significant reduction in the incidence of hepatic steatosis, fibrosis and HCC in the treated mir-122^{-/-} mice. Accordingly, the present disclosure provides a therapeutic for treating and/or preventing liver associated disorders, wherein the therapeutic comprises a delivery vehicle carrying a mir-122 gene. In some embodiments, the therapeutic comprises a mir-122 gene that is selected from the group consisting of human mir-122 gene and murine mir-122 gene. In some embodiments, the therapeutic comprises a delivery vehicle that is a vector, a liposome, a polymer, a pharmaceutically acceptable composition, or a device which facilitates delivery of such delivery vehicle.

In particular, the vector may be selected from the group consisting of adenovirus vectors, retrovirus vectors, adeno-associated virus vectors, herpes simplex virus vectors, SV40 vectors, polyoma virus vectors, papilloma virus vectors, picarnovirus vectors, vaccinia virus vectors, lentiviral vectors, alphaviral vectors, a helper-dependent adenovirus, and a plasmid.

The present disclosure provides that the therapeutic may be useful for treating and/or preventing liver associated disorders selected from the group consisting of hepatic steatosis, hepatitis, liver fibrosis, hepatocyte proliferation, and hepatocellular carcinoma.

A number of viral based systems have been developed for gene transfer into mammalian cells. For example, retroviruses provide a convenient platform for gene delivery systems. Selected sequences can be inserted into a vector and packaged in retroviral particles using techniques known in the art. The recombinant virus can then be isolated and delivered to cells of the subject either in vivo or ex vivo.

A number of adenovirus vectors have also been described. Unlike retroviruses which integrate into the host genome, adenoviruses persist extrachromosomally thus minimizing the risks associated with insertional mutagenesis.

Additionally, various adeno-associated virus (AAV) vector systems have been developed for gene delivery. AAV vectors can be readily constructed using techniques well known in the art.

Additional viral vectors which will find use for delivering the nucleic acid molecules encoding the mir-122 gene include those derived from the pox family of viruses, including vaccinia virus and avian poxyirus. Alternatively, avipoxyiruses, such as the fowlpox and canarypox viruses, can also be used to deliver the mir-122 gene.

Molecular conjugate vectors, such as the adenovirus chimeric vectors described in Michael et al., J. Biol. Chem. (1993) 268:6866-6869 and Wagner et al., Proc. Natl. Acad. Sci. USA (1992) 89:6099-6103, can also be used for gene delivery.

Members of the Alphavirus genus, such as, but not limited to, vectors derived from the Sindbis, Semliki Forest, and Venezuelan Equine Encephalitis viruses, will also find use as viral vectors for delivering the polynucleotides of the present disclosure.

A vaccinia based infection/transfection system can be conveniently used to provide for inducible, transient expression of the coding sequences of interest in a host cell. In this system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the polynucleotide of interest, driven by a T7 promoter. The polymerase expressed in the cytoplasm from the vaccinia virus recombinant transcribes the transfected DNA into RNA which is then translated into protein by the host translational machinery. The method provides for high level, transient, cytoplasmic production of large quantities of RNA and its translation products.

As an alternative approach to infection with vaccinia or avipox virus recombinants, or to the delivery of genes using other viral vectors, an amplification system that will lead to high-level expression following introduction into host cells can be used. Specifically, a T7 RNA polymerase promoter preceding the coding region for T7 RNA polymerase can be engineered. Translation of RNA derived from this template will generate T7 RNA polymerase which in turn will transcribe more template. Concomitantly, there will be a cDNA whose expression is under the control of the T7 promoter. Thus, some of the T7 RNA polymerase generated from translation of the amplification template RNA will lead to transcription of the desired gene. Because some T7 RNA polymerase is required to initiate the amplification, T7 RNA polymerase can be introduced into cells along with the template(s) to prime the transcription reaction. The polymerase can be introduced as a protein or on a plasmid encoding the RNA polymerase.

The synthetic expression cassettes of interest can also be delivered without a viral vector. For example, the synthetic expression cassettes can be packaged as DNA or RNA in liposomes prior to delivery to the subject or to cells derived therefrom. Lipid encapsulation is generally accomplished using liposomes which are able to stably bind or entrap and retain nucleic acid. The ratio of condensed DNA to lipid preparation can vary but will generally be around 1:1 (mg DNA:micromoles lipid), or more of lipid.

Liposomal preparations for use in the present disclosure include cationic (positively charged), anionic (negatively charged) and neutral preparations, with cationic liposomes particularly preferred. Cationic liposomes have been shown to mediate intracellular delivery of plasmid DNA, mRNA and purified transcription factors in functional form.

The liposomes can comprise multilammelar vesicles (MLVs), small unilamellar vesicles (SuVs), or large unilamellar vesicles (LUVs). The various liposome-nucleic acid complexes are prepared using methods known in the art.

The synthetic expression cassettes of interest may also be encapsulated, adsorbed to, or associated with, particulate carriers. Examples of particulate carriers include those derived from polymethyl methacrylate polymers, as well as microparticles derived from poly(lactides) and poly(lactide-co-glycolides), known as PLG.

Furthermore, other particulate systems and polymers can be used for the in vivo or ex vivo delivery of the gene of interest. For example, polymers such as polylysine, polyarginine, polyornithine, spermine, spermidine, as well as conjugates of these molecules, are useful for transferring a nucleic acid of interest. Similarly, DEAE dextran-mediated transfection, calcium phosphate precipitation or precipitation using other insoluble inorganic salts, such as strontium phosphate, aluminum silicates including bentonite and kaolin, chromic oxide, magnesium silicate, talc, and the like, will find use with the present methods.

Recombinant vectors carrying a synthetic expression cassette of the present disclosure are formulated into compositions for delivery to the subject. These compositions may either be prophylactic (to prevent disease) or therapeutic (to treat disease). The compositions will comprise a “therapeutically effective amount” of the gene of interest such that an amount of the gene of interest can be produced in vivo in the individual to which it is administered. The exact amount necessary will vary depending on the subject being treated; the age and general condition of the subject to be treated; the severity of the condition being treated; the particular gene selected and its mode of administration, among other factors. An appropriate effective amount can be readily determined by one of skill in the art. Thus, a “therapeutically effective amount” will fall in a relatively broad range that can be determined through routine experimentation.

The compositions will generally include one or more “pharmaceutically acceptable excipients or vehicles” such as water, saline, glycerol, polyethyleneglycol, hyaluronic acid, ethanol, etc. Additionally, auxiliary substances, such as wetting or emulsifying agents, pH buffering substances, surfactants and the like, may be present in such vehicles. Certain facilitators of immunogenicity or of nucleic acid uptake and/or expression can also be included in the compositions or coadministered, such as, but not limited to, bupivacaine, cardiotoxin and sucrose.

Compounds or treatments that have an effect on a mir-122-related disorder can be administered directly to the patient. Administration may be done by any of the routes normally used for introducing a compound into ultimate contact with the tissue to be treated. The compounds are administered in any suitable manner, preferably with pharmaceutically acceptable carriers. Suitable methods of administering such compounds are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions of the present disclosure.

Formulations suitable for parenteral administration, such as, for example, by intravenous, intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of the present disclosure, compositions can be administered, for example, by intravenous infusion, orally, topically, intraperitoneally, intravesically or intrathecally. The formulations of compounds can be presented in unit-dose or multi-dose sealed containers, such as ampules and vials. Injection solutions and suspensions can be prepared from sterile powders, granules, and tablets of the kind previously described.

The dose administered to a patient, in the context of the present disclosure should be sufficient to effect a beneficial therapeutic response in the patient over time. The dose will be determined by the efficacy of the particular compound employed and the condition of the patient, as well as the body weight or surface area of the patient to be treated. The size of the dose also will be determined by the existence, nature, and extent of any adverse side-effects that accompany the administration of a particular compound or vector in a particular patient.

The present disclosure further provides for a method of preventing and/or treating a liver associated disorder comprising administering to a subject in need thereof a therapeutically effective amount of the mir-122 gene.

In some embodiments, the method relates to preventing and/or treating a liver associated disorder selected from the group consisting of hepatic steatosis, hepatitis, liver fibrosis, hepatocyte proliferation, and hepatocellular carcinoma.

In some embodiments, the method comprises an administering step using a delivery vehicle. In some embodiments, the delivery vehicle is a vector, a liposome, a polymer, a pharmaceutically acceptable composition, or a device which facilitates delivery of such delivery vehicle. In some embodiments, the vector is selected from the group consisting of adenovirus vectors, retrovirus vectors, adeno-associated virus vectors, herpes simplex virus vectors, SV40 vectors, polyoma virus vectors, papilloma virus vectors, picarnovirus vectors, vaccinia virus vectors, lentiviral vectors, alphaviral vectors, a helper-dependent adenovirus, and a plasmid.

In some embodiments, the method includes administering in a manner selected from the group consisting of intravenous administration, subcutaneous administration, intra-bone marrow administration, intra-arterial administration, intra-cardiac administration, intracerebral administration, intraspinal administration, intra-peritoneal administration, intra-muscular administration, parenteral administration, intra-rectal administration, intra-tracheal injection, intra-nasal administration, intradermal administration, epidermal administration, oral administration and combinations thereof.

In some embodiments, the method includes administering to the mammal in need of treatment multiple therapeutically effective amounts of the mir-122 gene. In other embodiments, the method includes administering the mir-122 gene in combination with another therapeutic. Such additional therapeutic may include, but is not limited to, anticancer therapies or therapeutics, antiviral agents, anti-inflammatory agents, immunosuppressive agents, and anti-fibrotic agents.

In some embodiments, the method of preventing and/or treating a liver associated disorder comprising administering to a subject that is a human.

The results provided herein indicate that mir-122 deficiency is involved in liver cancer, providing a biological marker for the disease. Accordingly, the present disclosure further provides a method for detecting the presence or a predisposition to a liver associated disorder in a subject by detecting the level of mir-122 in a sample. In one embodiment, the method comprises the steps of: obtaining a test sample from the subject; determining the level of mir-122 expression in the test sample; comparing the mir-122 expression level from the test sample to the expression level present in a control sample known not to have, or not to be predisposed to a liver associated disorder, wherein an alteration in the level of mir-122 expression in the test sample as compared to the control sample indicates the presence or predisposition to a liver associated disorder. A decrease in the level of mir-122, as compared to the control standard, is indicative of the presence of or risk to develop a liver associated disorder. The liver associated disorder may be selected from the group consisting of hepatic steatosis, hepatitis, liver fibrosis, hepatocyte proliferation, and hepatocellular carcinoma.

The present disclosure also provides for a method for screening a candidate agent for the ability to treat and/or prevent liver associated disorder comprising: providing a transgenic knock-out non-human animal whose genome comprises a disruption in the endogenous mir-122 gene, wherein the animal exhibits an altered phenotype selected from the group consisting of hepatic steatosis, hepatitis, liver fibrosis, hepatocyte proliferation, and hepatocellular carcinoma; administering to the animal the candidate agent, and evaluating the animal to determine whether the candidate agent affects and/or ameliorates at least one of the altered phenotype.

In some embodiments, the candidate agent is a mir-122 target gene. In some embodiments, the mir-122 target gene is selected from the group consisting of AlpI, Cs, Ctgf, Igf2, Jun, Klf6, Prom1 and Sox4.

The present disclosure is further illustrated by the following specific examples. The examples are provided for illustration only and should not be construed as limiting the scope of the application in any way.

### EXAMPLES

### Example 1

### Construction of Targeting Vector and Generation of mir-122^{-/-} Mice

To explore the intrinsic roles of miR-122 in various aspects of liver biology, a mutant mouse strain with a germ-line deletion of mir-122 using homologous recombination was generated as described herein.

The BAC clone bMQ-418A13 (chr18: 65269984-65437465) containing the entire mmu-mir-122 locus was purchased from Geneservice (Cambridge, UK). A genomic fragment of 13 kb encompassing 7.8 kb upstream and 5.1 kb downstream of pre-mir-122 was cloned to PL253 in bacteria strain EL350 by recombineering-based method.

The genomic fragment of mir-122 constructed in PL253 was used to replace the wild-type allele of mir-122 in 129Sv mouse embryonic stem cells (MESC). MESC clones containing the targeted allele were identified by Southern blot analysis. Several clones were isolated and transfected together with a vector encoding the Cre recombinase to delete a fragment of 1544 bp containing the entire pre-mir-122. Clones with the mir-122 knockout allele were identified by Southern blot analysis and were injected into C57BL/6J blastocysts. Germline transmission of the mir-122^{-/-} allele was achieved by crossing the chimeric mice with normal C57BL/6 mice. The homozygous mir-122^{-/-} mice were generated with littermates by crossing the heterozygous offspring. Genotyping of the F1 and successive generations was performed by Southern blotting and by PCR.

Mice carrying the homozygous deletion of mir-122 (hereafter referred to as mir-122^{-/-} mice) were born at the expected Mendelian frequency. They were fertile and indistinguishable from their wild-type (WT) and heterozygous littermates.

The animal studies were conducted in accordance with the Guidelines for the Care and Use of Mammals in Neuroscience and Behavioral Research and were proved by Institutional Animal Care and Use Committee (IACUC) of National Yang-Ming University.

Mice carrying the homozygous deletion (hereafter referred to as mir-122^{-/-} mice) were born at the expected Mendelian frequency. They are fertile and are indistinguishable from their wild-type (WT) and heterozygous littermates.

### Example 2

### Pathophysiological Features of mir-122^{-/-} Mice

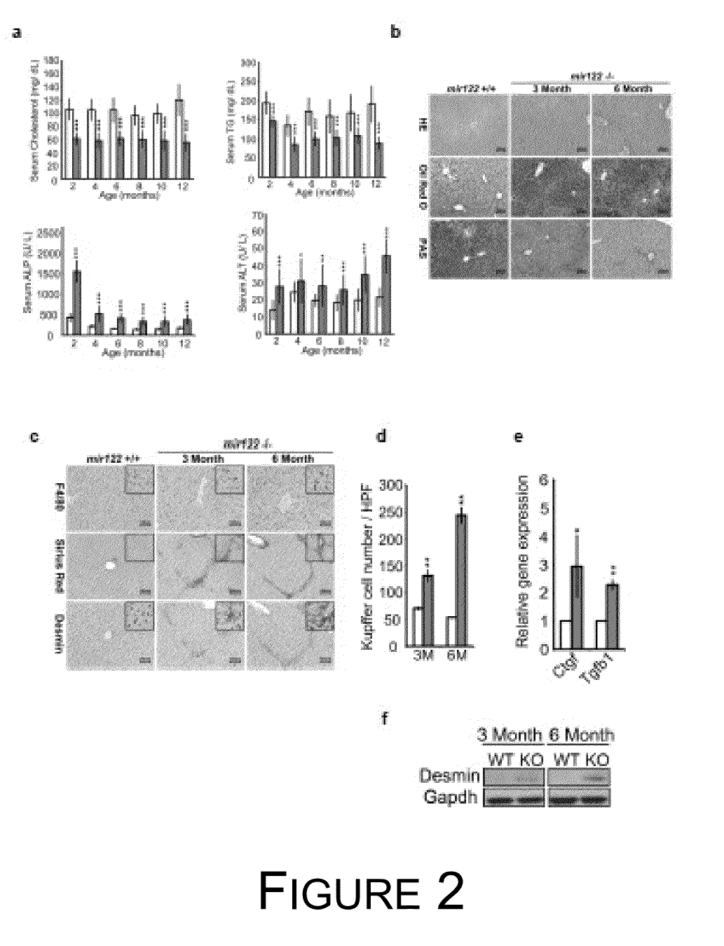

The somatic deletion of mir-122 led to significant reductions in serum cholesterol and triglyceride (TG), but the levels of alkaline phosphatase (ALP) and alanine transaminase (ALT) were found to be higher than those of WT mice (FIG. 2a). The trend toward a reduced serum cholesterol and TG in the mir-122^{-/-} mice is in agreement with but more pronounced than results reported for mice treated with anti-miR-122 oligomers.

Histological examinations of the livers of mir-122^{-/-} mice revealed extensive lipid accumulation and reduced glycogen storage (FIG. 2b), along with inflammation and fibrosis, when compared to WT controls. A strong positive reaction to anti-F4/80, an antibody for mouse macrophages and monocytes, was detected in the mir-122^{-/-} livers (FIGS. 2c, 2d). Portal fibrosis due to the activation of stellate cells was detected in the mir-122^{-/-} livers using Sirius Red staining and immuoreactivity with anti-desmin antibody (FIGS. 2c, 2f); this was accompanied by the elevated expression of two important fibrogenic factors, Ctgf and Tgfb1 (FIG. 2e).

### Example 3

### Liver Damage in mir-122^{-/-} Mice

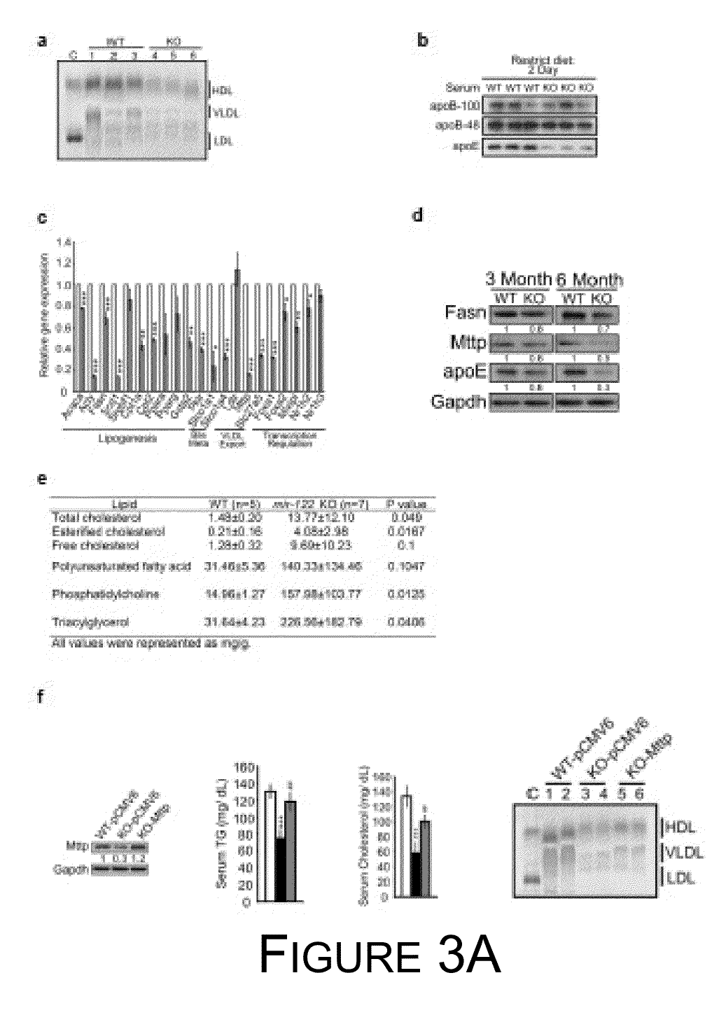

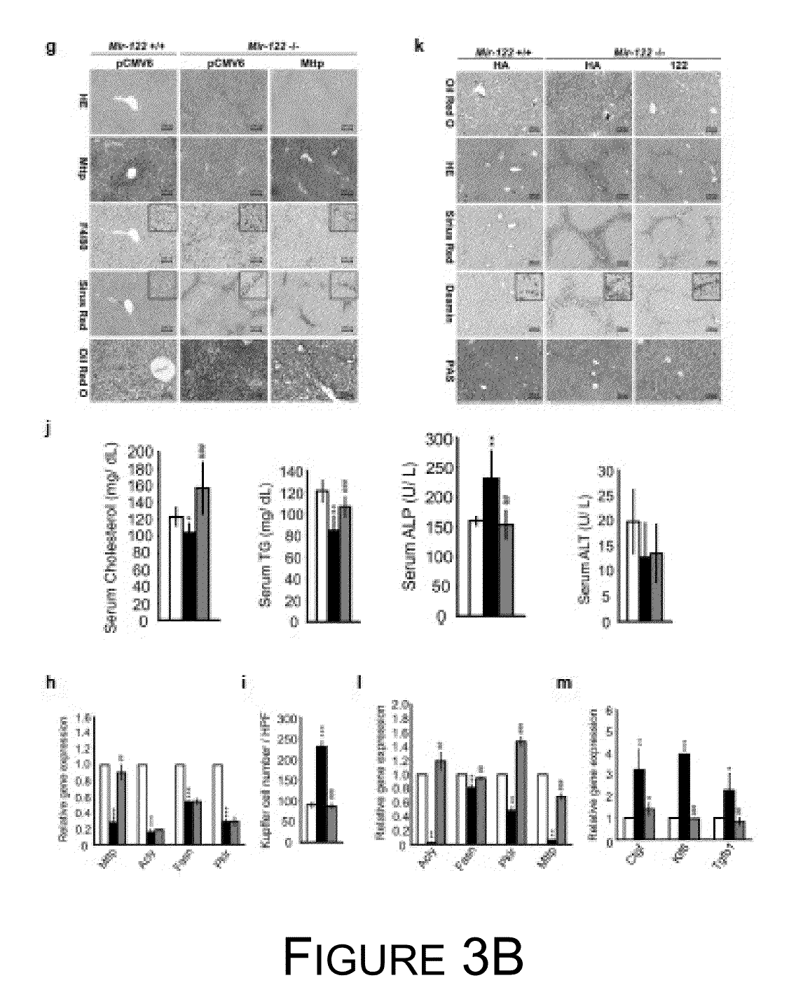

The coexistence of liver steatosis and low serum triglyceride and cholesterol levels in the mir-122^{-/-} mice necessitated an in-depth analysis of hepatic lipid metabolism. The levels of both serum HDL and VLDL were found to be significantly reduced in the mir-122^{-/-} mice (FIG. 3a) and were accompanied by lower levels of serum apoB-100 and apoE (FIG. 3b). Because the LDL levels were similar, it is unlikely that VLDL was converted to LDL in an accelerated manner or that hepatic LDL uptake was affected in the mir-122^{-/-} mice. These results strongly suggest that the detected disturbance was most likely due to the reduced hepatic secretion of the lipoproteins into the circulation.

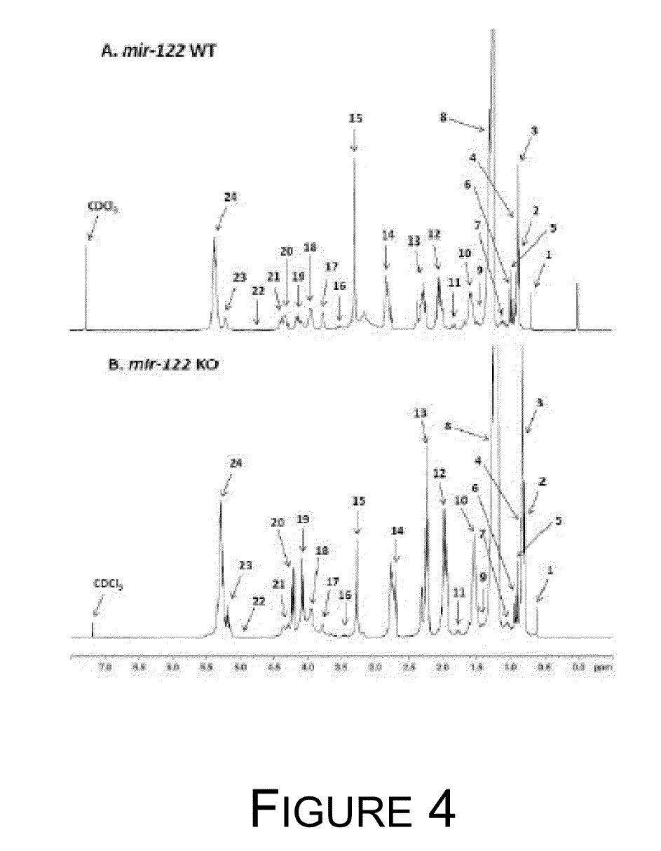

Hepatic VLDL assembly and secretion are dependent on sufficient amounts of apoB-100, microsomal triglyceride transfer protein (Mttp) and various lipids. To confirm the presence of a possible defect in the VLDL export, we analyzed the expression of various genes involved in lipid metabolism by RT-qPCR; the levels of the various lipid metabolites were analyzed via lipid profiling. Consistent with previous findings (Esau, C. et al., Cell Metab 3, 87-98 (2006); Krutzfeldt, J. et al., Nature 438, 685-9 (2005)), we demonstrated that there was a general downward trend of the gene expression of lipogenesis, bile acid metabolism, lipid transport and transcription regulation of lipid homeostasis in mir-122^{-/-} compared to WT mice (FIG. 3c). Notably, the expression of Mttp was significantly reduced at both the mRNA and protein levels (FIGS. 3c, 3d). Lipid profiling by ¹H-NMR spectroscopy was performed to determine targeted lipid metabolites. The amount of cholesterol (based on the signal intensity of H-18 at 0.68 ppm), TG (based on the proton signals and intensities of the C-1 and C-3 protons of TG glycerol skeleton) and phospatidylcholine was found to be significantly increased in the mir-122^{-/-} livers (FIG. 3e, p<0.05, FIG. 4, Supplementary Table 1).

SUPPLEMENTARY TABLE 1
Hepatic lipid contents in WT and mir-122^{-/-} mice determined by ¹H-NMR

| | Chemical | | | |
|---|---|---|---|---|
| | shifts | | mir-122 KO | |
| Assignment^{a} | (δ, ppm) | WT (n = 5)^{b} | (n = 7)^{b} | P value |
| Total cholesterol C-18, CH₃ | 0.693 − 0.664 | 1.48 ± 0.18 | 13.78 ± 12.10 | 0.0362 |
| Total cholesterol C-26, CH3/C-27, CH₃ | 0.867 − 0.838 | 7.43 ± 2.33 | 63.31 ± 44.47 | 0.0161 |
| Fatty acyl chain CH₃(CH₂)ₙ | 0.885 − 0.867 | 12.60 ± 1.51 | 76.99 ± 61.51 | 0.0325 |
| Total cholesterol C-21, CH₃ | 0.942 − 0.900 | 6.50 ± 1.31 | 50.25 ± 41.71 | 0.0323 |
| Free cholesterol C-19, CH₃ | 1.017 − 1.000 | 1.99 ± 0.25 | 11.76 ± 10.25 | 0.0453 |
| Esterified cholesterol C-19, CH₃ | 1.032 − 1.017 | 0.86 ± 0.17 | 11.89 ± 8.77 | 0.0158 |
| Multiple cholesterol protons | 1.185 − 1.059 | 6.55 ± 1.38 | 63.68 ± 49.59 | 0.0226 |
| Fatty acyl chain (CH₂)ₙ | 1.412 − 1.202 | 311.60 ± 44.61 | 2371.60 ± 1786.15 | 0.0225 |
| Multiple cholesterol protons | 1.522 − 1.419 | 5.73 ± 1.16 | 31.56 ± 31.12 | 0.0708 |
| Fatty acyl chain —CH₂CH₂CO | 1.666 − 1.524 | 32.68 ± 4.40 | 921.51 ± 1592.95 | 0.1903 |
| Multiple cholesterol protons | 1.904 − 1.789 | 2.44 ± 0.20 | 21.79 ± 18.69 | 0.0338 |
| Fatty acyl chain —CH₂CH═ | 2.151 − 1.966 | 170.23 ± 260.17 | 314.98 ± 208.98 | 0.3782 |
| Fatty acyl chain —CH₂CO | 2.358 − 2.213 | 33.15 ± 4.94 | 233.32 ± 199.56 | 0.0379 |
| Fatty acyl chain ═CHCH₂CH═ | 2.903 − 2.727 | 43.69 ± 6.65 | 194.89 ± 186.75 | 0.0762 |
| Sphingomyelin and choline N(CH₃)₃ | 3.402 − 3.238 | 23.08 ± 2.57 | 95.91 ± 81.46 | 0.0560 |
| Free cholesterol C-3, CH | 3.579 − 3.457 | 6.49 ± 1.20 | 21.90 ± 16.60 | 0.0501 |
| Phosphatidylcholine N—CH₂ | 3.826 − 3.693 | 7.89 ± 0.60 | 83.33 ± 54.73 | 0.0108 |
| Glycerophospholipid backbone C-3, CH₂ | 4.011 − 3.839 | 13.74 ± 1.10 | 101.70 ± 74.68 | 0.020 |
| Triacylglycerol backbone C-1, CH₂ | 4.203 − 4.029 | 15.16 ± 1.81 | 108.53 ± 87.56 | 0.0303 |
| Triacylglycerol backbone C-3, CH₂ | 4.386 − 4.247 | 13.37 ± 1.66 | 89.98 ± 95.45 | 0.0780 |
| Phosphatidylcholine PO—CH₂ | 4.444 − 4.386 | 2.71 ± 0.39 | 91.72 ± 97.52 | 0.0522 |
| Esterified cholesterol C-3, CH | 4.757 − 4.701 | 0.21 ± 0.15 | 4.08 ± 2.98 | 0.0138 |
| Glycerolphospholipid backbone C-2, CH | 5.244 − 5.147 | 11.58 ± 1.18 | 110.53 ± 184.90 | 0.2065 |
| Triacylglycerol backbone C-2, CH | 5.284 − 5.242 | 4.56 ± 1.45 | 59.43 ± 36.80 | 0.0076 |
| Fatty acyl chain —HC═CH— | 5.472 − 5.284 | 57.43 ± 7.05 | 368.24 ± 280.43 | 0.0263 |

^{a}Assignments of chemical shifts were based on authentic samples or values reported in the literature.
^{b}Signal intensities were used for quantitation. Data are shown as mean ± SD.

### Example 4

### Restoration of Mttp or mir-122 Expression Reduced Liver Pathology

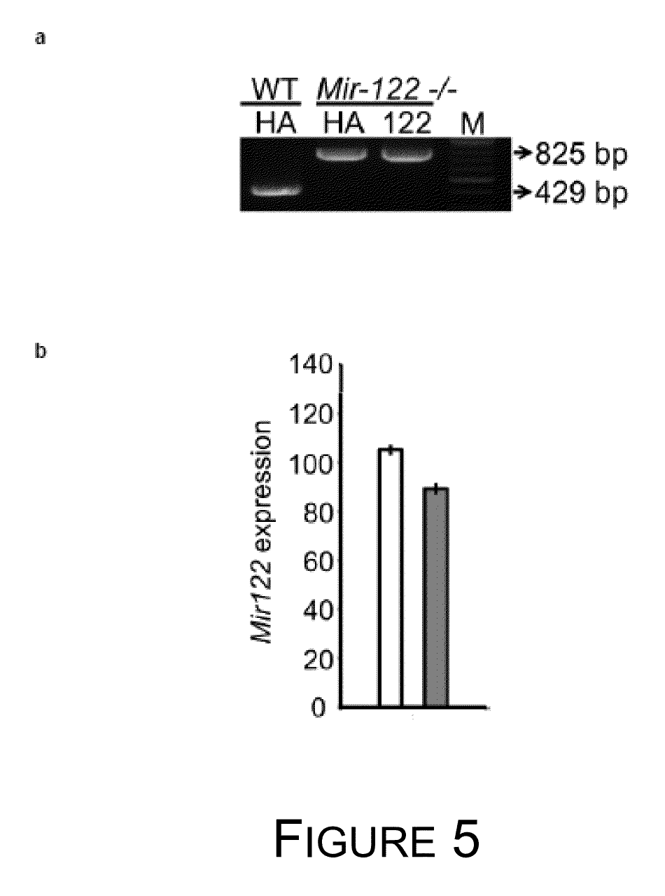

We tested whether restoration of mir-122 expression was able to reduce the presence of liver pathology. Sustained Mttp or mir-122 expression over one month in mir-122^{-/-} mice was achieved by the hydrodynamic injection 14 of Mttp or miR-122 expression vector (FIG. 3g, FIG. 5b).

The restoration of Mttp in mir-122^{-/-} specifically increased Mttp expression (FIGS. 3g, 3h), facilitated VLDL transport and normalized the serum levels of cholesterol and fasting triglyceride (FIG. 3f). The Mttp-restored livers displayed moderate hepatic steatosis, inflammation and fibrosis (FIGS. 3g, 3i).

In contrast to Mttp restoration, the re-expression of mir-122 changed a broad spectrum of biological activities, including the improved liver functions achieved by Mttp-restoration, elevated glycogen storage (FIG. 3k) and increased expression of various genes involved in lipid metabolism (Acyl, Fasn, Pklr, and Mttp) (FIG. 3l). The evidence of significantly fewer activated stellate cells and the suppression of elevated expression of three fibrogenic factors (Klf6, Tgfb1 and Ctgf) (FIGS. 3k, 3m) elucidated the anti-fibrotic capability of mir-122. This result supports the role of suppressed Mttp expression as the underlying defect of impaired VLDL assembly and hepatic steatosis in mir-122^{-/-} mice.

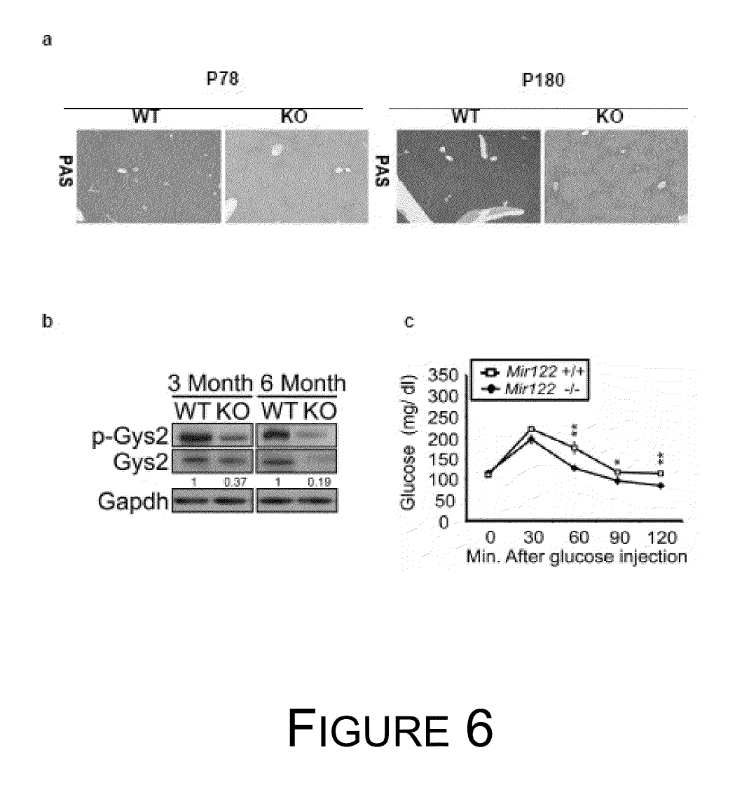

The pattern of low serum TG and high hepatic TG observed in mir-122^{-/-} mice was indicative of the impairment in MTTP and VLDL assembly found in patients infected with HCV genotype 3 and in Fatty Liver Shionogi (FLS) mice. Similar to FLS mice, mir-122^{-/-} mice experienced a slight impairment of glucose tolerance, although the serum glucose level was not significantly affected (FIG. 6c). The reduced expression of hepatic glycogen synthase (Gys2) can partially explain the inadequate glycogen accumulation (FIGS. 6a, 6b). Although the short-term inhibition of mir-122 has been shown to improve liver steatosis in mice fed with a high-fat diet1, our results revealed a close association in the long-term deficiency of mir-122 and metabolic diseases.

### Example 5

### miR-122 Downregulation Regulates Hepatocarcinogenesis

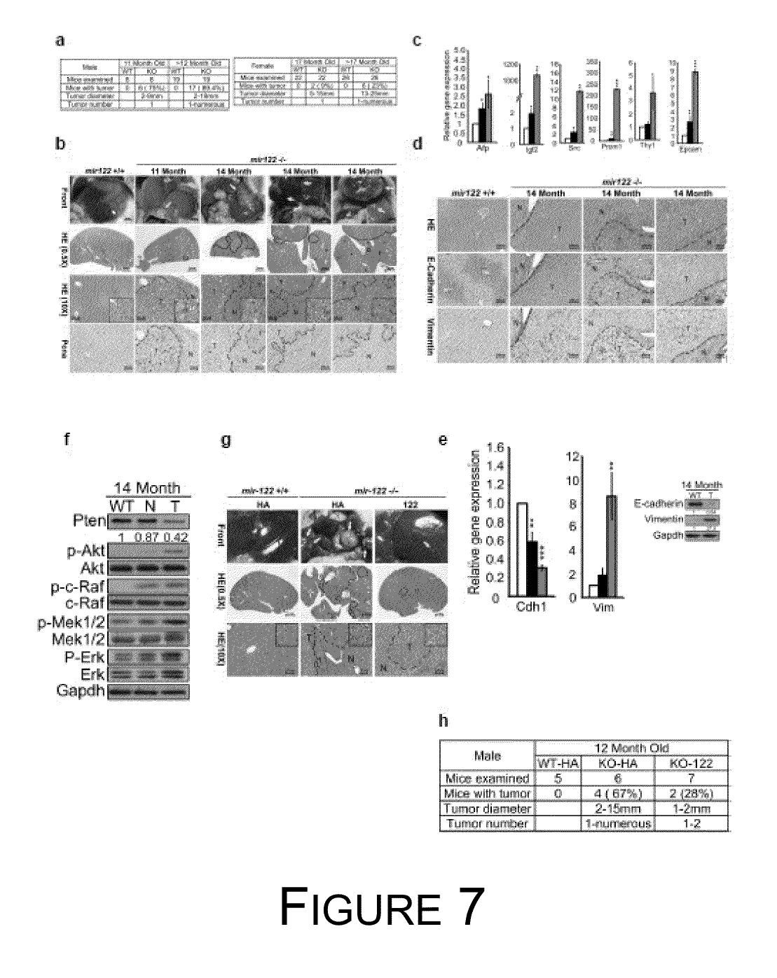

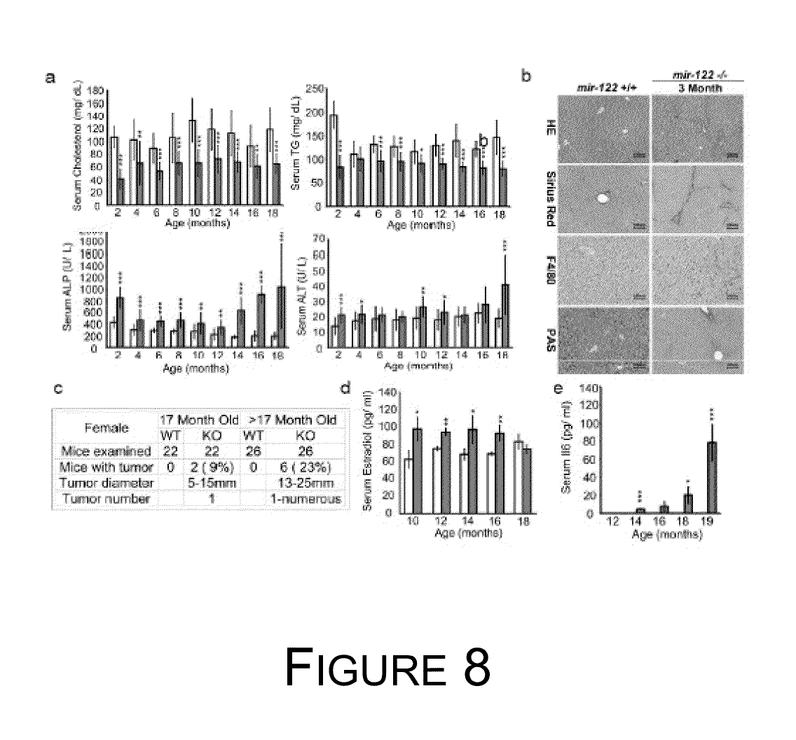

Liver lesions and hepatocellular carcinoma (HCC) developed in mir-122^{-/-} mice. A striking gender disparity in HCC of mir-122^{-/-} mice with a male-to-female ratio of 3.9:1 (89.4%:23%) (FIG. 7a) recapitulates the HCC incidence in humans. The female mir-122^{-/-} mice developed pathological features indistinguishable from the male counterpart, except in the delayed occurrence of HCC. Similar to human disease, a higher serum 116 level is a probable risk factor for HCC development in female mir-122^{-/-} mice (FIG. 8). A representative liver from an 11-month old mir-122^{-/-} male mouse revealed a single small round-shaped solid tumor, with a smoothly demarcated edge and uniform cell morphology that resembled a well-differentiated liver tumor (FIG. 7b, 2nd panel). However, three representative livers from 14-month old mir-122^{-/-} male mice showed multiple larger-sized tumors with invasion fronts (FIG. 7b, 3rd to 5th panel). There were marked cell pleomorphisms with the presence of occasional giant cells and fatty droplets that resembled poorly differentiated HCC cells (FIG. 7b insets). These tumors also exhibited rapid proliferation (FIG. 7b, Pcna IHC). The manifestations of multiple larger nodules and regions with invasive edges suggest that these tumors are malignant in nature.

The highly elevated expression of oncofetal genes, such as Afp, Igf2 and Src, as well as tumor-initiating cell markers, such as Prom1, Thy1 and Epcam, were also detected in these tumors (FIG. 7c).

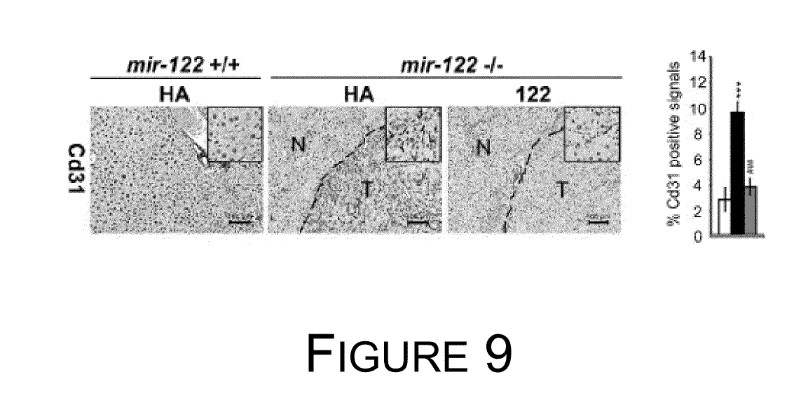

MiR-122 modulation of the epithelial-mesenchymal transition (EMT) has been demonstrated in human HCC cell lines, and the re-expression of miR-122 has been found to greatly reduce MAPK signaling and vimentin expression. This was accompanied by an inhibition in intrahepatic metastasis. The mir-122^{-/-} tumors not only showed molecular alterations that were compatible with EMT, namely, the loss of E-cadherin and the upregulation of vimentin (FIGS. 7d, 7e), but they also expressed less Pten protein and a strong activation of Akt and Mapk signaling (FIG. 7f). To establish the timing of hepatocarcinogenesis when the impact of the mir-122 deficiency took effect, we traced the outcome of the prolonged re-expression of mir-122 that was launched at 3 months of age. The continuous re-expression of mir-122 did not prevent tumor initiation but effectively impeded tumor progression, as reflected by the diminished tumor size (FIG. 7g), reduced tumor incidence (FIG. 7h) and re-differentiated features, i.e., smoothly demarcated edge, less nuclear pleomorphism (FIG. 7g) and reduced mean microvessel density (FIG. 9). The observation that hepatocytes with homozygous mir-122 deletions are prone to hepatocarcinogenesis suggests that endogenous mir-122 is a plausible guardian of hepatocyte differentiation.

### Example 6

### Gene Expression Analyses of mir-122^{-/-} Mice

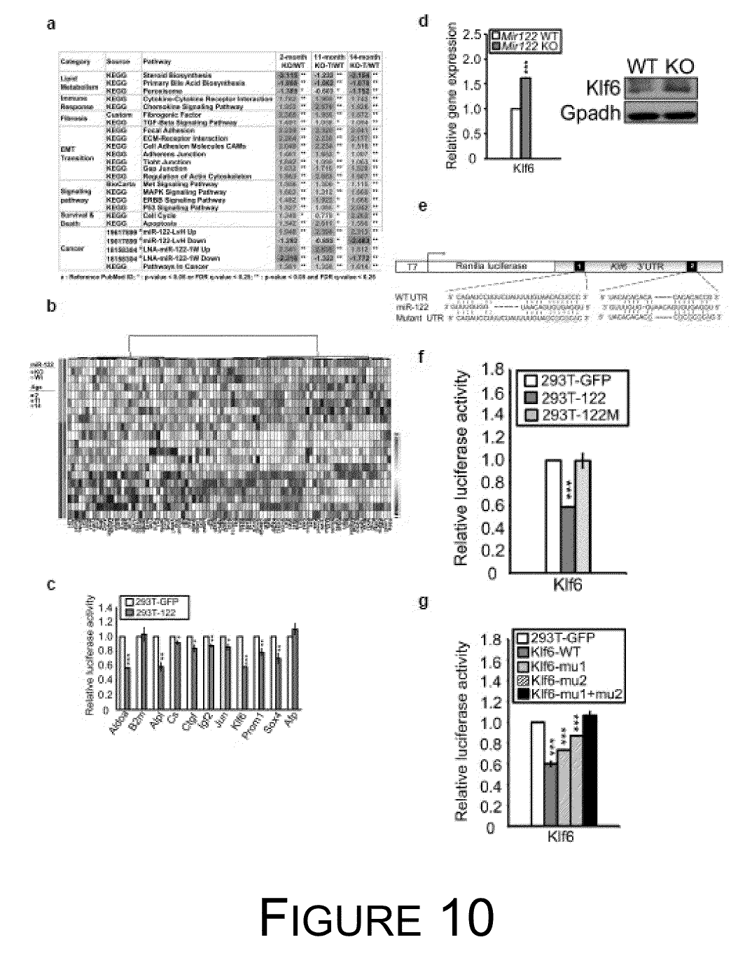

The pathogenic association between miR-122 deficiency and hepatic diseases may be multifactorial in nature. We next performed gene expression analyses of the liver tissues from 2-month-old mice and tumors from male mir-122^{-/-} mice to elucidate the pathway disturbance that drives cancer initiation and progression. Gene set enrichment analysis revealed that multiple pathways in the KEGG database were significantly modulated. Notably, three pathways involving steroid biosynthesis, bile acid biosynthesis and peroxisomes were de-enriched in the mir-122^{-/-} livers (FIG. 10a, Supplementary Table 2), which was in line with earlier reports on mice that were administered antisense oligomers (Esau, C. et al., Cell Metab 3, 87-98 (2006); Krutzfeldt, J. et al., Nature 438, 685-9 (2005); Elmen, J. et al., Nucleic Acids Res 36, 1153-62 (2008); Gatfield, D. et al., Genes Dev 23, 1313-26 (2009)).

SUPPLEMENTARY TABLE 2
Gene Set Enrichment Analysis (GSEA) analysis of 2-month-old male mir-122^{-/-}
and wild-type mice. The pathways are ranked by Nominal Enrichment Score (NES).

| | | Norminal | | | | | | |
|---|---|---|---|---|---|---|---|---|
| | | enrich- | | | Enriched | | Leading | |
| | | ment | | | in | | edge | |
| Pathway | | score | Nominal | FDR | Pheno- | List | gene | |
| ID | Map Name | (NES) | p-value | q-val | type | size | count | Leading edge Gene Symbol |
| *CGU00001 | Liver | 2.3036 | 0.0000 | 0.0000 | KO | 23 | 15 | Ddr1, Col1a2, Cygb, Ctgf, Klf6, Col1a1, Loxl1, Col3a1, |
| | fibrosis | | | | | | | Col6a3, Pdgfra, Pdgfd, Timp1, Pdgfrb, |
| | | | | | | | | Col6a2, Vcam1 |
| MMU04510 | Focal | 2.2310 | 0.0000 | 0.0000 | KO | 194 | 92 | Itgb8, Col1a2, Spp1, Ccnd1, Col1a1, Cav1, Itga6, |
| | adhesion | | | | | | | Col3a1, Thbs1, Col5a2, Pik3r5, Col6a1, Hgf, Pak1, |
| | | | | | | | | Lama2, Pdgfra, Pdgfd, Lamc3, Pdgfrb, Prkca, Itga8, |
| | | | | | | | | Actg1, Src, Col6a2, Parvb, Mapk3, Vwf, Zyx, Jun, |
| | | | | | | | | Col4a2, Lamb2, Thbs2, Lamb1 - 1, Col4a1, Vegfc, |
| | | | | | | | | Comp, Rac2, Pak3, Myl2, Bcl2, Pdgfa, Flna, Bad, |
| | | | | | | | | Vasp, Cav2, Myl12b, Itga4, Myl9, Ppp1cc, Lama1, |
| | | | | | | | | Pak6, Pik3r3, Shc4, Pdgfc, Sos2, Ccnd2, Fyn, Ctnnb1, |
| | | | | | | | | Pik3cd, Met, 2900073G15Rik, Itgb3, Prkcb, Rock2, |
| | | | | | | | | Birc2, Pik3cg, Vav1, Pdgfb, Myl10, Pak2, Myl7, Lama5, |
| | | | | | | | | Diap1, Rap1b, Vav3, Crkl, Parvg, Dock1, Lama4, |
| | | | | | | | | Pik3ca, Itga2, Col4a4, Rac3, Igf1r, Ilk, Flt1, Mapk1, |
| | | | | | | | | Erbb2, Gsk3b, Lamc1, Itgb4, Mylpf |
| MMU04512 | ECM-receptor | 2.2023 | 0.0000 | 0.0003 | KO | 81 | 22 | Itgb8, Col1a2, Spp1, Col1a1, Itga6, Col3a1, Thbs1, |
| | interaction | | | | | | | Col5a2, Col6a1, Lama2, Cd44, Lamc3, Itga8, Col6a2, |
| | | | | | | | | Vwf, Col4a2, Lamb2, Thbs2, Lamb1 - 1, Col4a1, Npnt, |
| | | | | | | | | Comp |
| MMU05150 | Staphylococcus | 2.1141 | 0.0000 | 0.0002 | KO | 47 | 21 | Ighg, H2 - Ab1, H2 - Eb1, H2 - Aa, Itgb2, H2 - Oa, |
| | aureus | | | | | | | Masp1, Fcgr3, Fpr2, Icam1, C3ar1, Selplg, Fpr1, |
| | infection | | | | | | | Itgam, H2 - Ob, C5ar1, H2 - DMb2, Fcgr1, Cfd, C1qc, |
| | | | | | | | | H2 - DMa |
| MMU05140 | Leishmaniasis | 2.0904 | 0.0000 | 0.0004 | KO | 65 | 29 | Ighg, H2 - Ab1, H2 - Eb1, H2 - Aa, Cyba, Tgfb3Tgfb2, |
| | | | | | | | | Itgb2, H2 - Oa, Tlr2, Mapk3, Jun, Fcgr3, |
| | | | | | | | | Ncf1, Mapk13, Itgam, Marcksl1, Itga4, Tlr4, H2 - Ob, |
| | | | | | | | | H2 - DMb2, Ncf4, Fcgr1, Nfkbia, H2 - DMa, Prkcb, |
| | | | | | | | | Irak4, Jak1, Nfkbib |
| MMU04514 | Cell adhesion | 2.0583 | 0.0000 | 0.0006 | KO | 130 | 43 | Cldn7, Itgb8, Cd34, Itga6, H2 - Ab1, H2 - Eb1, Ocln, |
| | molecules | | | | | | | H2 - Aa, Cldn8, Itgb2, H2 - Oa, Itga8, Sell, Cldn2, |
| | (CAMs) | | | | | | | Vcam1, Jam2, Cd2, Pvrl1, Cldn6, Icam1, Selplg, |
| | | | | | | | | Cldn23, Cd28, Itgam, Alcam, Cd99, Itga4, Pvrl2, Cd40, |
| | | | | | | | | H2 - Ob, Cd86, Cntnap1, H2 - DMb2, Cd276, Nlgn1, |
| | | | | | | | | Sdc1, Nrxn2, Icam2, H2 - DMa, Cdh1, Negr1, Glg1, |
| | | | | | | | | Pecam1 |
| MMU04145 | Phagosome | 1.9737 | 0.0000 | 0.0033 | KO | 147 | 43 | Sftpd, Vamp3, Coro1a, Cd14, Cybb, Atp6v0e2, Ighg, |
| | | | | | | | | H2 - Ab1, Thbs1, H2 - Eb1, Cd209a, H2 - Aa, Cyba, |
| | | | | | | | | Itgb2, H2 - Oa, Tlr2, Actg1, Marco, Tubb2b, Thbs2, |
| | | | | | | | | Fcgr3, Ncf1, Comp, Atp6v0e, Dync1li2, Atp6v0a2, |
| | | | | | | | | Atp6v1b2, Cd209d, Itgam, Tuba1a, Mrc2, Tlr4, H2 - |
| | | | | | | | | Ob, Atp6v0d2, Mpo, H2 - DMb2, Ncf4, Rab5c, Fcgr1, |
| | | | | | | | | Atp6v1g2, H2 - DMa, Itgb3, Ctss |
| MMU05146 | Amoebiasis | 1.9417 | 0.0000 | 0.0041 | KO | 107 | 31 | Serpinb1a, Col1a2, Serpinb6b, Cd14, Col1a1, Col3a1, |
| | | | | | | | | Ighg, Col5a2, Pik3r5, Gna14, Lama2, Tgfb3, Tgfb2, |
| | | | | | | | | Lamc3, Itgb2, Tlr2, Prkca, Arg2, Serpinb6a, Col4a2, |
| | | | | | | | | Lamb2, Lamb1 - 1, Col4a1, Itgam, Lama1, Pik3r3, |
| | | | | | | | | Tlr4, Gna15, Il1r2, Rab5c, Pik3cd |
| MMU05310 | Asthma | 1.9043 | 0.0016 | 0.0054 | KO | 25 | 12 | Ighg, H2 - Ab1, H2 - Eb1, H2 - Aa, H2 - Oa, Prg2, |
| | | | | | | | | Cd40, H2-Ob, H2-DMb2, H2-DMa, Il5, Fcer1g |
| MMU04810 | Regulation | 1.8791 | 0.0000 | 0.0070 | KO | 207 | 77 | Itgb8, Gsn, Cd14, Itga6, Fgf21, Pik3r5, Pak1, Pdgfra, |
| | of actin | | | | | | | Pdgfd, Fgfr1, Pip4k2a, Ezr, Was, Itgax, Itgb2, Pdgfrb, |
| | cytoskeleton | | | | | | | Itga8, Actg1, Arhgef7, Mapk3, Ssh3, Cyfip2, Mras, |
| | | | | | | | | Fgd1, Rac2, Pak3, Myl2, Pdgfa, Pfn2, Itgam, Myl12b, |
| | | | | | | | | Myh14, Itga4, Myl9, Ppp1cc, Pak6, Pip4k2c, Myh9, |
| | | | | | | | | Pik3r3, Pdgfc, Fgf8, Sos2, Bdkrb2, Git1, Rras, |
| | | | | | | | | Nckap1l, Pik3cd, 2900073G15Rik, Itgb3, Fgf18, |
| | | | | | | | | Chrm3, Tmsb4x, Enah, Rock2, Pip4k2b, Pik3cg, |
| | | | | | | | | Fgf13, Fgf2, Vav1, Pdgfb, Msn, Myl10, Pak2, Myl7, |
| | | | | | | | | Csk, Fgf12, Diap1, Itgal, Vav3, Crkl, Dock1, Fgf9, |
| | | | | | | | | Arpc1b, Slc9a1, Pik3ca, Itqa2, Rac3 |
| MMU05340 | Primary | 1.8557 | 0.0016 | 0.0088 | KO | 34 | 22 | Blnk, Ighg, Tnfrsf13b, Ada, Cd3d, Ikbkg, Cd40, Jak3, |
| | immuno- | | | | | | | Rfx5, Rfxank, Cd8b1, Rag1, Btk, Tap2, Zap70, |
| | deficiency | | | | | | | Dclre1c, Aicda, Cd8a, Tap1, Ciita, Tnfrsf13c, Il7r |
| MMU04530 | Tight | 1.8385 | 0.0000 | 0.0092 | KO | 129 | 43 | Cldn7, Ocln, Cldn8, Prkca, Cldn2, Actg1, Src, Amotl1, |
| | junction | | | | | | | Mras, Jam2, Pard6b, Inadl, Cldn6, Hcls1, Prkch, Myl2, |
| | | | | | | | | Cldn23, Gnai1, Myl12b, Cdk4, Myh14, Myl9, Epb4.1l1, |
| | | | | | | | | Ppp2r2a, Myh9, Llgl1, Pard6g, Rras, Ctnnb1, Gnai2, |
| | | | | | | | | Cask, 2900073G15Rik, Yes1, Rab13, Prkcb, Epb4.1l2, |
| | | | | | | | | Pard6a, Myh2, Myl10, Myh1, Myl7, Ppp2r2b, Cttn |
| MMU05414 | Dilated | 1.8295 | 0.0000 | 0.0092 | KO | 88 | 21 | Itgb8, Lmna, Itga6, Ighg, Tpm4, Lama2, Tgfb3, Tgfb2, |
| | cardio- | | | | | | | Tnnt2, Sgcb, Itga8, Actg1, Adcy6, Myl2, Cacna2d4, |
| | myopathy | | | | | | | Adcy7, Itga4, Tpm1, Itgb3, Cacnb2, Atp2a2 |
| | (DCM) | | | | | | | |
| MU05211 | Renal cell | 1.8245 | 0.0000 | 0.0091 | KO | 71 | 25 | Pik3r5, Hgf, Pak1, Tgfb3, Tgfb2, Mapk3, Jun, Vegfc, |
| | carcinoma | | | | | | | Slc2a1, Pak3, Pdgfa, Pak6, Pik3r3, Gab1, Sos2, Ets1, |
| | | | | | | | | Pik3cd, Met, Ep300, Pik3cg, Egln3, Pdgfb, Pak2, |
| | | | | | | | | Rap1b, Crkl |
| MMU05142 | Chagas | 1.8021 | 0.0000 | 0.0118 | KO | 103 | 41 | Pik3r5, Gna14, Tgfb3, Tgfb2, Tlr2, Ccl2, Mapk3, Cd3g, |
| | disease | | | | | | | Jun, Tgfbr1, Ccl12, Mapk13, Smad3, Cd3d, Gnai1, |
| | | | | | | | | Serpine1, Ppp2r2a, Ikbkg, Pik3r3, Tlr4, Bdkrb2, |
| | | | | | | | | Gna15, Tnfrsf1a, Nfkbia, Pik3cd, Gnai2, C1qc, Ccl5, |
| | | | | | | | | Pik3cg, Irak4, Il6, Gnaq, Ppp2r2b, Ticam1, Ifngr1, |
| | | | | | | | | Tgfbr2, Pik3ca, C1qa, Il1b, Plcb4, Ppp2r1a |
| MMU05100 | Bacterial | 1.7824 | 0.0015 | 0.0136 | KO | 69 | 26 | Cav1, Pik3r5, Was, Clta, Actg1, Src, 4631416L12Rik, |
| | invasion of | | | | | | | Hcls1, Mad2l2, Cav2, Pik3r3, Gab1, Shc4, Cd2ap, |
| | epithelial | | | | | | | Ctnnb1, Pik3cd, Met, Cdh1, Cblb, Pik3cg, Crkl, Cttn, |
| | cells | | | | | | | Dock1, Elmo1, Arpc1b, Pik3ca |
| MMU05145 | Toxo- | 1.7776 | 0.0000 | 0.0140 | KO | 124 | 46 | Itga6, H2 - Ab1, H2 - Eb1, Pik3r5, H2 - Aa, Lama2, |
| | plasmosis | | | | | | | Tgfb3, Hspa1a, Tgfb2, Lamc3, H2 - Oa, Tlr2, Mapk3, |
| | | | | | | | | Lamb2, Il10rb, Lamb1 - 1, Mapk13, Bcl2, Bad, |
| | | | | | | | | Hspa1b, Gnai1, Lama1, Ikbkg, Cd40, Pik3r3, Tlr4, |
| | | | | | | | | Pla2g2f, H2 - Ob, Hspa2, Tnfrsf1a, H2 - DMb2, Nfkbia, |
| | | | | | | | | Pik3cd, Gnai2, H2 - DMa, Birc2, Pik3cg, Irak4, Jak1, |
| | | | | | | | | Nfkbib, Pla2g3, Hspa8, Lama5, Ifngr1, Map2k6, Lama4 |
| MMU05144 | Malaria | 1.7687 | 0.0000 | 0.0147 | KO | 46 | 18 | Thbs1, Hgf, Tgfb3, Tgfb2, Itgb2, Tlr2, Ccl2, Vcam1, |
| | | | | | | | | Thbs2, Ccl12, Comp, Icam1, Cd40, Tlr4, Met, Sdc1, |
| | | | | | | | | Pecam1, Il6 |
| MMU04060 | Cytokine- | 1.7496 | 0.0000 | 0.0160 | KO | 220 | 56 | Ltb, Cxcl14, Cxcr7, Il1rap, Cxcr4, Pf4, Cxcl13, Cx3cr1, |
| | cytokine | | | | | | | Hgf, Prlr, Pdgfra, Pdgfd, Ccr1, Tgfb3, Tgfb2, Ccr2, |
| | receptor | | | | | | | Osmr, Tnfrsf12a, Cxcl16, Pdgfrb, Ccl2, Cxcl10, Tgfbr1, |
| | Interaction | | | | | | | Il10rb, Ccl12, Csf2ra, Vegfc, Tnfrsf1b, Inhbe, Ccl19, |
| | | | | | | | | Tnfrsf13b, Ccl27a, Cxcl12, Cx3cl1, Pdgfa, Flt3l, Cxcr2, |
| | | | | | | | | Bmpr1b, Csf2rb2, Tnfrsf21, Clcf1, Cd40, Cxcr6, Pdgfc, |
| | | | | | | | | Csf2rb, Ccl6, Ccl8, Cxcr3, Il1r2, Tnfrsf1a, Lepr, |
| | | | | | | | | Bmpr1a, Cxcr5, Ccr3, Met, Il17ra |
| MMU05214 | Glioma | 1.7253 | 0.0014 | 0.0200 | KO | 63 | 20 | Ccnd1, Pik3r5, Camk2b, Pdgfra, Pdgfrb, Prkca, |
| | | | | | | | | Mapk3, Calm3, Pdgfa, Cdk4, Pik3r3, Shc4, Sos2, |
| | | | | | | | | Cdkn1a, Pik3cd, Plcq2, Prkcb, Pik3cg, Pdgfb, Camk2a |
| MMU05218 | Melanoma | 1.7236 | 0.0029 | 0.0196 | KO | 71 | 24 | Ccnd1, Fgf21, Pik3r5, Hgf, Pdgfra, Pdgfd, |
| | | | | | | | | Fgfr1, Pdgfrb, Mapk3, Pdgfa, Bad, Cdk4, Pik3r3, Pdgfc, |
| | | | | | | | | Fgf8, Cdkn1a, Pik3cd, Met, Fgf18, Cdh1, Pik3cg, |
| | | | | | | | | Fgf13, Fgf2, Pdgfb |
| MMU05410 | Hypertrophic | 1.7094 | 0.0000 | 0.0221 | KO | 81 | 20 | Itgb8, Lmna, Itga6, Tpm4, Lama2, Tgfb3, Tgfb2, |
| | cardio- | | | | | | | Tnnt2, Sgcb, Itga8, Actg1, Myl2, Cacna2d4, Itga4, |
| | myopathy | | | | | | | Tpm1, Itgb3, Cacnb2, Atp2a2, Il6, Ryr2 |
| | (HCM) | | | | | | | |
| MMU00290 | Valine, | 1.6918 | 0.0140 | 0.0253 | KO | 10 | 9 | Lars2, Lars, Pdha2, Iars, Bcat1, Iars2, Vars, Vars2, |
| | leucine and | | | | | | | Pdha1 |
| | isoleucine | | | | | | | |
| | biosynthesis | | | | | | | |
| MMU05220 | Chronic | 1.6750 | 0.0029 | 0.0282 | KO | 72 | 22 | Ccnd1, Pik3r5, Tgfb3, Tgfb2, Mapk3, Ctbp2, Tgfbr1, |
| | myeloid | | | | | | | Smad3, Bad, Cdk4, Gab2, Ikbkg, Hdac2, Pik3r3, Shc4, |
| | leukemia | | | | | | | Sos2, Bcr, Cdkn1a, Nfkbia, Pik3cd, Cblb, Pik3cq |
| MMU04540 | Gap | 1.6304 | 0.0028 | 0.0413 | KO | 81 | 29 | Gja1, Pdgfra, Pdgfd, Pdgfrb, Prkca, Src, Mapk3, |
| | junction | | | | | | | Tubb2b, Adcy6, Adcy7, Pdgfa, Tuba1a, Gnai1, Pdgfc, |
| | | | | | | | | Sos2, Lpar1, Gnai2, Itpr3, Prkcb, Pdgfb, Gnaq, |
| | | | | | | | | Gucy1b3, Tuba8, Gucy1a3, Prkacb, Cdk1, Prkg1, |
| | | | | | | | | Plcb4, Grm5 |
| MMU05322 | Systemic | 1.6289 | 0.0028 | 0.0406 | KO | 74 | 19 | Ighg, H2 - Ab1, H2 - Eb1, H2 - Aa, H2 - Oa, |
| | lupus | | | | | | | Hist2h3c2, Hist1h3f, Fcgr3, Cd28, Cd40, Hist3h2a, |
| | erythema- | | | | | | | H2 - Ob, Cd86, H2 - DMb2, Fcgr1, Hist2h3c1, C1qc, |
| | tosus | | | | | | | H2 - DMa, Hist1h2be |
| MMU05222 | Small cell | 1.6066 | 0.0054 | 0.0488 | KO | 83 | 32 | Ccnd1, Itga6, Pik3r5, Lama2, Lamc3, Col4a2, Lamb2, |
| | lung cancer | | | | | | | Lamb1 - 1, Col4a1, Bcl2, Fhit, Cdk4, Lama1, Ikbkg, |
| | | | | | | | | Pik3r3, Nfkbia, Pik3cd, Apaf1, Birc2, Pik3cg, Lama5, |
| | | | | | | | | Ccne1, Ccne2, Traf1, Lama4, Pik3ca, Itga2, Col4a4, |
| | | | | | | | | Casp9, Cks1b, Lamc1, Cdkn2b |
| MMU04010 | MAPK | 1.6015 | 0.0000 | 0.0496 | KO | 261 | 86 | Relb, Cd14, Fgf21, Mapkapk3, Pak1, Pdgfra, Tgfb3, |
| | signaling | | | | | | | Ddit3, Fgfr1, Hspa1a, Tgfb2, Pdgfrb, Prkca, Mapk3, |
| | pathway | | | | | | | Mknk2, Mapkapk2, Jun, Mras, Ntf3, Tgfbr1, Mapk13, |
| | | | | | | | | Map4k4, Cdc25b, Rac2, Cacna2d4, Pdgfa, Flna, |
| | | | | | | | | Map3k1, Dusp6, Srf, Rps6ka3, Hspa1b, Ikbkg, Rasa2, |
| | | | | | | | | Fgf8, Pla2g2f, Sos2, Arrb1, Hspa2, Il1r2, Tnfrsf1a, |
| | | | | | | | | Rras, Map3k12, Mef2c, Rps6ka1, Stk3, Gadd45b, |
| | | | | | | | | Map3k3, Ngf, Fgf18, Cacnb2, Mapkapk5, |
| | | | | | | | | 2010110P09Rik, Prkcb, Fgf13, Fgf2, Pdgfb, Pla2g3, |
| | | | | | | | | Ppm1a, Arrb2, Rasgrp4, Pak2, Map3k8, Nfkb2, Hspa8, |
| | | | | | | | | Map3k5, Fgf12, Rap1b, Mapk8ip1, Crkl, Mapk8ip3, |
| | | | | | | | | Fgf9, Map2k6, Rps6ka6, Prkacb, Ptprr, Tgfbr2, Il1b, |
| | | | | | | | | Nfatc2, Rac3, Cacna1b, Nf1, Dusp5, Mapk1, Taok3, |
| | | | | | | | | Ntrk1 |
| MMU05215 | Prostate | 1.5962 | 0.0043 | 0.0507 | KO | 89 | 33 | Ccnd1, Pik3r5, Pdgfra, Pdgfd, Fgfr1, Pdgfrb, Mapk3, |
| | cancer | | | | | | | Bcl2, Pdgfa, Bad, Ikbkg, Pik3r3, Pdgfc, Sos2, Cdkn1a, |
| | | | | | | | | Ctnnb1, Nfkbia, Pik3cd, Ep300, Pik3cg, Pdgfb, |
| | | | | | | | | Creb3l2, Creb1, Hsp90aa1, Hsp90ab1, Ccne1, Ccne2, |
| | | | | | | | | Pik3ca, Igf1r, Casp9, Mapk1, Erbb2, Gsk3b |
| MMU05416 | Viral | 1.5949 | 0.0083 | 0.0501 | KO | 71 | 26 | Ccnd1, Cav1, Ighg, H2 - Ab1, H2 - Eb1, H2 - Aa, |
| | myocarditis | | | | | | | Lama2, Itgb2, H2 - Oa, Sgcb, Actg1, Rac2, Icam1, |
| | | | | | | | | Cd28, Myh14, Myh9, Cd40, H2 - Ob, Cd86, Fyn, |
| | | | | | | | | H2 - DMb2, Cd55, H2 - DMa, Cxadr, Myh2, Myh1 |
| MMU05412 | Arrhythmo- | 1.5868 | 0.0071 | 0.0527 | KO | 73 | 15 | Itgb8, Lmna, Itga6, Gja1, Lama2, Sgcb, Itga8, Actg1, |
| | genic | | | | | | | Cacna2d4, Jup, Itga4, Ctnnb1, Itgb3, Cacnb2, Atp2a2 |
| | right | | | | | | | |
| | ventricular | | | | | | | |
| | cardio- | | | | | | | |
| | myopathy | | | | | | | |
| | (ARVC) | | | | | | | |
| MMU05213 | Endometrial | 1.5659 | 0.0231 | 0.0622 | KO | 51 | 19 | Ccnd1, Pik3r5, Mlh1, Mapk3, Bad, Foxo3, Pik3r3, |
| | cancer | | | | | | | Sos2, Ctnnb1, Pik3cd, Axin2, Cdh1, Pik3cg, Pik3ca, |
| | | | | | | | | Casp9, Ilk, Mapk1, Erbb2, Gsk3b |
| MMU05200 | Pathways | 1.5638 | 0.0000 | 0.0620 | KO | 316 | 104 | Ccnd1, Mmp2, Itga6, Fgf21, Pik3r5, Hgf, Mlh1, |
| | in cancer | | | | | | | Lama2, Pdgfra, Tgfb3, Fgfr1, Tgfb2, Lamc3, Pdgfrb, |
| | | | | | | | | Prkca, Ralb, Hhip, Mapk3, Ctbp2, Jun, Col4a2, |
| | | | | | | | | Lamb2, Tgfbr1, Lamb1 - 1, Csf2ra, Col4a1, Vegfc, |
| | | | | | | | | Rac2, Slc2a1, Smad3, Jup, Bcl2, Pdgfa, Flt3l, Bad, |
| | | | | | | | | Wnt8a, Cdk4, Lama1, Ikbkg, Hdac2, Ppard, Pik3r3, |
| | | | | | | | | Fgf8, Sos2, Rassf5, Bcr, Cdkn1a, Ctnnb1, Nfkbia, |
| | | | | | | | | Pik3cd, Met, Fzd1, Mmp9, Ep300, Msh6, Dcc, Fgf18, |
| | | | | | | | | Ralbp1, Axin2, Cdh1, Plcg2, Cblb, Prkcb, Birc2, |
| | | | | | | | | Pik3cg, Egln3, Runx1t1, Fgf13, Il6, Fgf2, Pdgfb, Jak1, |
| | | | | | | | | Nfkb2, Lama5, Fgf12, Hsp90aa1, Hsp90ab1, Sfpi1, |
| | | | | | | | | Ccne1, Fzd2, Ccne2, Crkl, Wnt4, Traf1, Fgf9, Lama4, |
| | | | | | | | | Tgfbr2, Pik3ca, Wnt2b, Itga2, Col4a4, Rac3, Pparg, |
| | | | | | | | | Igf1r, Casp9, Mapk1, Erbb2, Cks1b, Csf3r, Gsk3b, |
| | | | | | | | | Lamc1, Vhl, Rassf1, Ntrk1 |
| MMU00600 | Sphingolipid | 1.5585 | 0.0176 | 0.0619 | KO | 38 | 13 | Gal3st1, Sptlc2, B4galt6, Degs2, Neu1, Smpd3, Glb1, |
| | metabolism | | | | | | | Sgpl1, Ppap2a, Ppap2c, Ugt8a, Neu3, Acer2 |
| MMU04210 | Apoptosis | 1.5491 | 0.0057 | 0.0652 | KO | 85 | 27 | Prkar2b, Il1rap, Pik3r5, Casp12, Endod1, Bcl2, Bad, |
| | | | | | | | | Csf2rb2, Capn1, Ikbkg, Pik3r3, Csf2rb, Tnfrsf1a, |
| | | | | | | | | Nfkbia, Pik3cd, Irak2, Apaf1, Ngf, 2010110P09Rik, |
| | | | | | | | | Birc2, Pik3cg, Irak4, Ripk1, Prkacb, Pik3ca, Il1b, |
| | | | | | | | | Casp9 |
| MMU05210 | Colorectal | 1.5484 | 0.0105 | 0.0641 | KO | 62 | 25 | Ccnd1, Pik3r5, Mlh1, Tgfb3, Tgfb2, Mapk3, Jun, |
| | cancer | | | | | | | Tgfbr1, Rac2, Smad3, Bcl2, Bad, Pik3r3, Ctnnb1, |
| | | | | | | | | Pik3cd, Msh6, Dcc, Axin2, Pik3cg, Tgfbr2, |
| | | | | | | | | Pik3ca, Rac3, Casp9, Mapk1, Gsk3b |
| MMU04350 | TGF-beta | 1.5311 | 0.0136 | 0.0712 | KO | 80 | 30 | Thbs1, Tgfb3, Tgfb2, Bmp6, Mapk3, Thbs2, Tgfbr1, |
| | signaling | | | | | | | Acvr1c, Comp, Inhbe, Smad3, Bmpr1b, Id1, Dcn, |
| | pathway | | | | | | | Bmpr1a, Ep300, Lefty2, Rock2, Bmp5, Lefty1, Bmpr2, |
| | | | | | | | | Rbl2, Tgfbr2, Ppp2r1a, Mapk1, Ifng, Rbl1, Smurf2, |
| | | | | | | | | Bmp8b, Cdkn2b |
| MMU00590 | Arachidonic | 1.5256 | 0.0157 | 0.0732 | KO | 73 | 8 | Cyp2b13, Gpx7, Cbr3, Ptgds, Cyp4a14, Gpx3, |
| | acid | | | | | | | Cyp4f16, Cyp4a31 |
| | metabolism | | | | | | | |
| MMU04115 | p53 | 1.5217 | 0.0191 | 0.0740 | KO | 64 | 22 | Ccnd1, Thbs1, Ccng1, Ccnb2, Igfbp3, Chek2, |
| | signaling | | | | | | | Zmat3, Cdk4, Serpine1, Ccnd2, Rrm2b, Mdm4, |
| | pathway | | | | | | | Cdkn1a, Gadd45b, Apaf1, Sesn2, Ddb2, |
| | | | | | | | | Pmaip1, Sesn3, Ccne1, Steap3, Ccne2 |
| MMU04012 | ErbB | 1.5058 | 0.0161 | 0.0790 | KO | 86 | 42 | Pik3r5, Pak1, Camk2b, Ereg, Prkca, Src, Mapk3, Jun, |
| | signaling | | | | | | | Btc, Pak3, Bad, Pak6, Pik3r3, Gab1, Shc4, Sos2, |
| | pathway | | | | | | | Cdkn1a, Pik3cd, Plcg2, Cblb, Prkcb, Pik3cg, |
| | | | | | | | | Camk2a, Pak2, Nck1, Crkl, Pik3ca, Mapk1, Erbb2, |
| | | | | | | | | Gsk3b, Erbb4, Nrg3, Pak4, Hbegf, Abl2, Map2k1, |
| | | | | | | | | Camk2d, Eif4ebp1, Mapk9, Nras, Cbl, Grb2 |
| MMU05212 | Pancreatic | 1.4749 | 0.0273 | 0.0981 | KO | 70 | 26 | Ccnd1, Pik3r5, Tgfb3, Tgfb2, Ralb, Mapk3, Tgfbr1, |
| | cancer | | | | | | | Vegfc, Rac2, Smad3, Pld1, Bad, Cdk4, Ikbkg, Pik3r3, |
| | | | | | | | | Pik3cd, Ralbp1, Pik3cg, Jak1, Tgfbr2, Pik3ca, Rac3, |
| | | | | | | | | Casp9, Mapk1, Erbb2, Arhgef6 |
| MMU00604 | Glycosphingo- | 1.4602 | 0.0637 | 0.1071 | KO | 15 | 5 | St3gal2, Hexb, St3gal5, Glb1, St6galnac4 |
| | lipid | | | | | | | |
| | biosynthesis - | | | | | | | |
| | ganglio | | | | | | | |
| | series | | | | | | | |
| MMU04520 | Adherens | 1.4422 | 0.0393 | 0.1181 | KO | 73 | 24 | Fgfr1, Was, Actg1, Src, Mapk3, Snai2, Tgfbr1, Pvrl1, |
| | junction | | | | | | | Rac2, Smad3, Pvrl2, Snai1, Fyn, Ctnnb1, Met, |
| | | | | | | | | Ep300, Cdh1, Yes1, Ptprm, Tgfbr2, Rac3, Igf3r, |
| | | | | | | | | Mapk1, Erbb2 |
| MMU00603 | Glycosphingo- | 1.4309 | 0.0961 | 0.1239 | KO | 15 | 6 | St3gal2, Hexb, Fut1, Sec1, A4galt, Naga |
| | lipid | | | | | | | |
| | biosynthesis - | | | | | | | |
| | globo series | | | | | | | |
| MMU04114 | Oocyte | 1.4304 | 0.0253 | 0.1223 | KO | 109 | 37 | Rec8, Cpeb1, Camk2b, Ccnb2, Mapk3, Adcy6, |
| | meiosis | | | | | | | Calm3, Spdye4, Adcy7, Mad2l2, Cdc20, Rps6ka3, |
| | | | | | | | | Anapc10, Ywhab, Ppp1cc, Anapc1, Espl1, Rps6ka1, |
| | | | | | | | | Itpr3, Ywhah, Ywhag, 2010110P09Rik, Camk2a, |
| | | | | | | | | Ywhaq, Ccne1, Ccne2, Rps6ka6, Prkacb, Cdk1, |
| | | | | | | | | Ppp2r1a, Ywhaz, Igf1r, Anapc4, Mapk1, Ppp1ca, |
| | | | | | | | | Ccnb1, Mad2l1 |
| MMU00601 | Glycosphingo- | 1.4270 | 0.0740 | 0.1214 | KO | 25 | 10 | B3galt2, Fut1, Gcnt2, B4galt4, St3gal4, Sec1, |
| | lipid | | | | | | | Ggta1, B4galt1, B4galt2, Fut4 |
| | biosynthesis - | | | | | | | |
| | lacto and | | | | | | | |
| | neolacto | | | | | | | |
| | series | | | | | | | |
| MMU05223 | Non-small | 1.4224 | 0.0516 | 0.1233 | KO | 54 | 20 | Ccnd1, Pik3r5, Prkca, Mapk3, Bad, Fhit, Foxo3, |
| | cell lung | | | | | | | Cdk4, Pik3r3, Sos2, Rassf5, Pik3cd, Plcg2, Prkcb, |
| | cancer | | | | | | | Pik3cg, Pik3ca, Casp9, Mapk1, Erbb2, Rassf1 |
| MMU00565 | Ether lipid | 1.3998 | 0.0706 | 0.1380 | KO | 34 | 11 | Pafah1b3, Pld1, Pafah1b2, Ppap2a, Ppap2c, Lpcat2, |
| | metabolism | | | | | | | Pla2g2f, Lpcat1, Pafah1b1, Pla2g3, Cept1 |
| MMU04370 | VEGF | 1.3982 | 0.0388 | 0.1356 | KO | 75 | 22 | Pik3r5, Mapkapk3, Prkca, Src, Mapk3, Mapkapk2, |
| | signaling | | | | | | | Mapk13, Rac2, Bad, Pik3r3, Pla2g2f, Pik3cd, Plcg2, |
| | pathway | | | | | | | 2010110P09Rik, Prkcb, Pik3cg, Pla2g3, Pik3ca, |
| | | | | | | | | Nfatc2, Rac3, Casp9, Mapk1 |
| MMU04144 | Endocytosis | 1.3711 | 0.0218 | 0.1601 | KO | 200 | 65 | Cav1, Nedd4l, Cxcr4, Fam125b, Pdgfra, Tgfb3, |
| | | | | | | | | Chmp4c, Hspa1a, Tgfb2, Clta, Src, Vps4a, Tgfbr1, |
| | | | | | | | | Pard6b, Rab31, Ehd2, Ehd4, Smad3, Pld1, Cxcr2, |
| | | | | | | | | Cav2, Hspa1b, Ehd1, Vps37c, Adrb2, Arrb1, Hspa2, |
| | | | | | | | | Pard6g, Git1, Rab5c, Rab11fip5, Ap2b1, Arap3, Met, |
| | | | | | | | | Chmp1a, Rab11fip3, Zfyve20, Agap1, Cblb, Psd4, |
| | | | | | | | | Vps24, Sh3kbp1, Rab11b, Pard6a, Il2rb, Arrb2, |
| | | | | | | | | Smap1, Hspa8, Ap2a2, Arfgap1, Tgfbr2, Sh3gl1, |
| | | | | | | | | Igf1r, Tfrc, Agap2, Pld2, Flt1, Ntrk1, Erbb4, Iqsec3, |
| | | | | | | | | Smurf2, Prkci, Rab22a, Fgfr4, Pip5k1c |
| MMU03430 | Mismatch | 1.3375 | 0.1347 | 0.1923 | KO | 22 | 13 | Mlh1, Rpa2, Exo1, Msh6, Rfc2, Pold4, Pcna, |
| | repair | | | | | | | Lig1, Pold2, Pold3, Rfc3, Pms2, Rpa1 |
| MMU00480 | Glutathione | 1.3320 | 0.0854 | 0.1963 | KO | 52 | 11 | Gpx7, Gpx3, Mgst2, Gstm2, Gstm3, Ggt6, |
| | metabolism | | | | | | | Rrm2b, Gstm5, Mgst3, G6pdx, Ggt5 |
| MMU04110 | Cell cycle | 1.3303 | 0.0446 | 0.1954 | KO | 122 | 45 | Ccnd1, Tgfb3, Ccnb2, Tgfb2, Cdkn2c, Cdkn1c, |
| | | | | | | | | Cdc25b, Fzr1, Smad3, Chek2, Mad2l2, Cdc20, |
| | | | | | | | | Anapc10, Cdk4, Orc2l, Ywhab, Bub1b, Hdac2, |
| | | | | | | | | Ccnd2, Anapc1, Cdkn1a, Espl1, Ep300, |
| | | | | | | | | Gadd45b, Ywhah, Ywhag, Cdc25a, Ywhaq, |
| | | | | | | | | Cdc14a, Ccna2, Cdk7, Pcna, Ccne1, Ccne2, |
| | | | | | | | | Rbl2, Orc3l, Cdk1, Ywhaz, Anapc4, Rad21, |
| | | | | | | | | Gsk3b, Rbl1, Cdkn2b, Ccnb1, Mad2l1 |
| MMU00790 | Folate | 1.3081 | 0.1615 | 0.2169 | KO | 11 | 1 | Alpl |
| | biosynthesis | | | | | | | |
| MMU00750 | Vitamin B6 | 1.2872 | 0.1537 | 0.2365 | KO | 6 | 3 | Psat1, Pnpo, Pdxp |
| | metabolism | | | | | | | |
| MMU05219 | Bladder | 1.2871 | 0.1234 | 0.2338 | KO | 41 | 9 | Ccnd1, Mmp2, Thbs1, Mapk3, Vegfc, Cdk4, Cdkn1a, |
| | cancer | | | | | | | Mmp9, Cdh1 |
| MMU00982 | Drug | 1.2867 | 0.1149 | 0.2315 | KO | 65 | 7 | Cyp2b13, Fmo3, Fmo2, Mgst2, Fmo4, Gstm2, Gstm3 |
| | metabolism - | | | | | | | |
| | Cytochrome | | | | | | | |
| | P450 | | | | | | | |
| MMU00520 | Amino sugar | 1.2801 | 0.1216 | 0.2374 | KO | 46 | 13 | Gnpda2, Gnpda1, Uap1l1, Hexb, Nagk, Hk1, Gmppa, |
| | and | | | | | | | Pgm1, Pgm3, Npl, Gfpt1, Gne, Mpi |
| | nucleotide | | | | | | | |
| | sugar | | | | | | | |
| | metabolism | | | | | | | |
| MMU04130 | SNARE | 1.2785 | 0.1357 | 0.2368 | KO | 35 | 8 | Vamp3, Stx6, Bet1l, Snap29, Stx11, Stx2, Gosr2, |
| | interactions | | | | | | | Ykt6 |
| | in vesicular | | | | | | | |
| | transport | | | | | | | |
| MMU04146 | Peroxisome | −1.3769 | 0.0400 | 0.3918 | WT | 78 | 30 | Slc27a2, Nudt12, Pex11c, Pex1, Pxmp2, Cat, |
| | | | | | | | | Dhrs4, Acox2, Mlycd, Ehhadh, Acox3, Acsl5, |
| | | | | | | | | Amacr, Ephx2, Dao, Pex11a, Pex16, Hsd17b4, |
| | | | | | | | | Sod2, Acsl6, Hao1, Decr2, Pecr, Mpv17l, Mvk, |
| | | | | | | | | Acaa1b, Abcd2, Idh1, Pmvk, Scp2 |
| MMU00900 | Terpenoid | −1.8799 | 0.0026 | 0.0158 | WT | 14 | 8 | Mvk, Mvd, Acat2, Fdps, Idi1, Pmvk, Hmgcr, Hmgcs1 |
| | backbone | | | | | | | |
| | biosynthesis | | | | | | | |
| MMU00120 | Primary | −1.9265 | 0.0024 | 0.0108 | WT | 15 | 9 | Cyp8b1, Cyp46a1, Baat, Slc27a5, Acox2, Cyp27a1, |
| | bile acid | | | | | | | Amacr, Hsd17b4, Cyp7a1 |
| | biosynthesis | | | | | | | |
| MMU00100 | Steroid | −2.1391 | 0.0000 | 0.0017 | WT | 18 | 3 | Cel, Hsd17b7, Sqle |
| | biosynthesis | | | | | | | |

*CGU00001: A curated list of liver fibrotic genes (Gutiérrez-Ruiz, et al., 2007; Friedman 2008; Bosselut et al., 2010)
NES: normalized enrichment score with positive and negative enrichment scores indicating correlation and anti-correlation with mir-122 knockout phenotype, respectively.
FDR: false detection rate.
P: nominal P value.

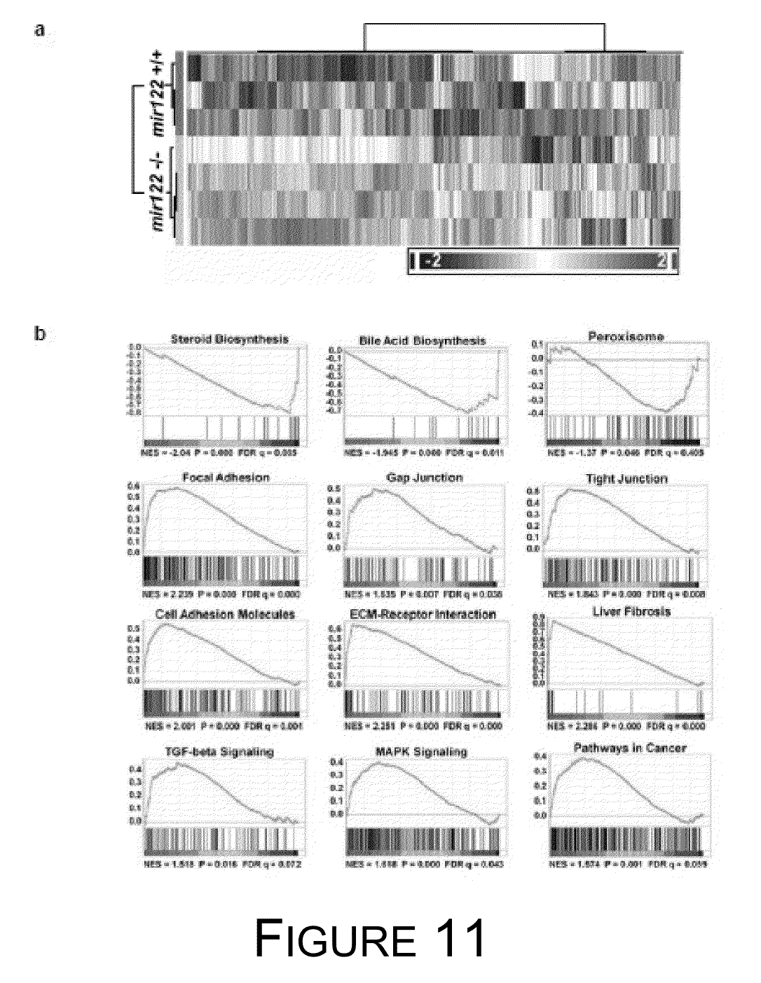

The significantly enriched pathways of all age groups involved immune response, EMT transition, fibrogenic pathways, signal transduction, survival and death, and cancer phenotypes (FIG. 10a). These results provide a clear molecular explanation of the fibrotic phenotype observed in the mir-122^{-/-} mice, as TGF-beta signaling is an important contributor to liver fibrosis and the disruption of cell-cell interaction is a hallmark of liver fibrosis. In addition, the enrichment patterns observed in the curated gene sets from hepatoma patients with high versus low miR-122 levels confirmed that the pathway disturbance observed in the mir-122^{-/-} mice closely resembled that of human HCC. Although there were no histological signs of precancerous lesions with younger samples, the enriched pathways clearly indicated that dysregulation in the livers were instigated in young mir-122^{-/-} mice (FIG. 11, Supplementary Tables 3, 4).

SUPPLEMENTARY TABLE 3
Differentially expressed genes in mir-122^{-/-} mice livers.
KO/WT, Expression ratio between mir-122^{-/-} and WT (−1.5 ≦ KO/WT ≧ 1.5).

| Probeset | Symbol | KO/WT | Probeset | Symbol | KO/WT | Probeset | Symbol | KO/WT |
|---|---|---|---|---|---|---|---|---|
| 1448194_a_at | H19 | 635.58 | 1439560_x_at | Gm5480 | 4.96 | 1450611_at | Orm3 | 3.32 |
| 1419590_at | Cyp2b9 | 43.71 | 1460550_at | Mtmr11 | 4.81 | 1456873_at | Clic5 | 3.27 |
| 1448152_at | Igf2 | 34.1 | 1459740_s_at | Ucp2 | 4.65 | 1433883_at | Tpm4 | 3.26 |
| 1433966_x_at | Asns | 26.83 | 1424959_at | Anxa13 | 4.64 | 1428055_at | Rian | 3.25 |
| 1452905_at | Meg3 | 23.61 | 1417399_at | Gas6 | 4.64 | 1424126_at | Alas1 | 3.23 |
| 1427747_a_at | Lcn2 | 17.4 | 1419700_a_at | Prom1 | 4.64 | 1451978_at | Loxl1 | 3.23 |
| 1418712_at | Cdc42ep5 | 13.42 | 1448416_at | Mgp | 4.5 | 1416046_a_at | Fuca2 | 3.22 |
| 1458442_at | Al132709 | 12.46 | 1418449_at | Lad1 | 4.42 | 1455162_at | Ttc39a | 3.22 |
| 1419394_s_at | S100a8 | 11.28 | 1425837_a_at | Ccrn4l | 4.4 | 1420378_at | Sftpd | 3.2 |
| 1449479_at | Cyp2b13 | 9.87 | 1452463_x_at | Gm10883 | 4.34 | 1455955_s_at | Snx17 | 3.15 |
| 1435196_at | Ntrk2 | 9.28 | 1421430_at | Rad51l1 | 4.29 | 1456388_at | Atp11a | 3.13 |
| 1448837_at | Vil1 | 9.05 | 1417419_at | Ccnd1 | 4.28 | 1444139_at | Ddit4l | 3.12 |
| 1428223_at | Mfsd2a | 8.66 | 1453435_a_at | Fmo2 | 4.18 | 1427963_s_at | Rdh9 | 3.12 |
| 1420438_at | Orm2 | 8.06 | 1423611_at | Alpl | 4.09 | 1433916_at | Vamp3 | 3.11 |
| 1417821_at | D17H6S56E-5 | 7.8 | 1425120_x_at | Ifi27l2b | 4.07 | 1415919_at | Npdc1 | 3.1 |
| 1441102_at | Prlr | 7.62 | 1424305_at | Igj | 4.07 | 1426302_at | Tmprss4 | 3.08 |
| 1425394_at | BC023105 | 7.38 | 1424007_at | Gdf10 | 3.86 | 1416953_at | Ctgf | 3.07 |
| 1433610_at | AA986860 | 6.92 | 1436643_x_at | Hamp2 | 3.85 | 1460351_at | S100a11 | 3.01 |
| 1424649_a_at | Tspan8 | 6.78 | 1451780_at | Blnk | 3.79 | 1418962_at | Necap2 | 2.97 |
| 1433575_at | Sox4 | 6.73 | 1416596_at | Slc44a4 | 3.78 | 1426519_at | P4ha1 | 2.96 |
| 1460406_at | Pls1 | 6.65 | 1430172_a_at | Cyp4f16 | 3.75 | 1423607_at | Lum | 2.94 |
| 1456226_x_at | Ddr1 | 6.55 | 1455431_at | Slc5a1 | 3.69 | 1448735_at | Cp | 2.92 |
| 1448182_a_at | Cd24a | 6.54 | 1424477_at | Tmem184a | 3.68 | 1460361_at | 5033414D02Rik | 2.9 |
| 1448595_a_at | Bex1 | 6.41 | 1416579_a_at | Epcam | 3.63 | 1433816_at | Mcart1 | 2.9 |
| 1433744_at | Lrtm2 | 6.21 | 1436991_x_at | Gsn | 3.62 | 1433924_at | Peg3 | 2.89 |
| 1427178_at | Tmc4 | 5.91 | 1423669_at | Col1a1 | 3.59 | 1418511_at | Dpt | 2.86 |
| 1416666_at | Serpine2 | 5.81 | 1416108_a_at | Tmed3 | 3.59 | 1436890_at | Uap1l1 | 2.86 |
| 1417836_at | Gpx7 | 5.79 | 1416114_at | Sparcl1 | 3.5 | 1416529_at | Emp1 | 2.84 |
| 1429523_a_at | Slc39a5 | 5.72 | 1431146_a_at | Cpne8 | 3.49 | 1448393_at | Cldn7 | 2.83 |
| 1427357_at | Cda | 5.69 | 1423933_a_at | 1600029D21Rik | 3.45 | 1434418_at | Lass6 | 2.83 |
| 1423484_at | Bicc1 | 5.68 | 1416432_at | Pfkfb3 | 3.44 | 1438377_x_at | Slc13a3 | 2.83 |
| 1449254_at | Spp1 | 5.68 | 1439375_x_at | Aldoa | 3.41 | 1427386_at | Arhgef16 | 2.81 |
| 1427020_at | Scara3 | 5.62 | 1423707_at | Tmem50b | 3.41 | 1434089_at | Synpo | 2.8 |
| 1457030_at | Mirg | 5.39 | 1420911_a_at | Mfge8 | 3.39 | 1423630_at | Cygb | 2.79 |
| 1416646_at | Afp | 4.98 | 1419573_a_at | Lgals1 | 3.34 | 1448770_a_at | Atpif1 | 2.78 |
| 1438625_s_at | Cdk16 | 2.78 | 1417178_at | Gipc2 | 2.49 | 1426750_at | Flnb | 2.22 |
| 1450717_at | Ang | 2.76 | 1426529_a_at | Tagln2 | 2.49 | 1428640_at | Hsf2bp | 2.22 |
| 1417664_a_at | Ndrg3 | 2.75 | 1421917_at | Pdgfra | 2.47 | 1460243_at | Sptlc2 | 2.22 |
| 1428066_at | Ccdc120 | 2.74 | 1435067_at | B230208H17Rik | 2.46 | 1449145_a_at | Cav1 | 2.21 |
| 1427883_a_at | Col3a1 | 2.73 | 1417409_at | Jun | 2.46 | 1455099_at | Mogat2 | 2.21 |
| 1437056_x_at | Crispld2 | 2.73 | 1428306_at | Ddit4 | 2.44 | 1434944_at | Dmpk | 2.19 |
| 1428316_a_at | Fundc2 | 2.73 | 1418444_a_at | Gde1 | 2.43 | 1424229_at | Dyrk3 | 2.19 |
| 1424131_at | Col6a3 | 2.72 | 1427201_at | Mustn1 | 2.43 | 1419132_at | Tlr2 | 2.19 |
| 1417116_at | Slc6a8 | 2.72 | 1425567_a_at | Anxa5 | 2.41 | 1426910_at | Pawr | 2.18 |
| 1434891_at | Ptgfrn | 2.71 | 1439389_s_at | Myadm | 2.38 | 1452016_at | Alox5ap | 2.17 |
| 1452649_at | Rtn4 | 2.7 | 1435156_at | BC046331 | 2.36 | 1425896_a_at | Fbn1 | 2.17 |
| 1416517_at | Pnpla6 | 2.69 | 1429570_at | Mlkl | 2.36 | 1428715_at | Gfpt1 | 2.16 |
| 1424962_at | Tm4sf4 | 2.69 | 1452227_at | Sel1l3 | 2.36 | 1435525_at | Kctd17 | 2.16 |
| 1425764_a_at | Bcat2 | 2.68 | 1416535_at | Mcrs1 | 2.35 | 1435254_at | Plxnb1 | 2.16 |
| 1423217_a_at | Fam32a | 2.67 | 1425921_a_at | 1810055G02Rik | 2.33 | 1426397_at | Tgfbr2 | 2.16 |
| 1454078_a_at | Gal3st1 | 2.67 | 1451997_at | Zfp426 | 2.33 | 1416096_at | Vipar | 2.16 |
| 1450850_at | Ezr | 2.66 | 1417231_at | Cldn2 | 2.32 | 1416656_at | Clic1 | 2.15 |
| 1433521_at | Ankrd13c | 2.64 | 1448111_at | Ctps2 | 2.32 | 1450857_a_at | Col1a2 | 2.15 |
| 1435682_at | Lars2 | 2.64 | 1460644_at | Bckdk | 2.31 | 1433796_at | Endod1 | 2.15 |
| 1453572_a_at | Plp2 | 2.64 | 1422501_s_at | Idh3a | 2.31 | 1417156_at | Krt19 | 2.13 |
| 1436223_at | Itgb8 | 2.62 | 1421375_a_at | S100a6 | 2.31 | 1449851_at | Per1 | 2.13 |
| 1436902_x_at | Tmsb10 | 2.62 | 1455269_a_at | Coro1a | 2.3 | 1427231_at | Robo1 | 2.13 |
| 1424927_at | Glipr1 | 2.61 | 1416164_at | Fbln5 | 2.3 | 1419569_a_at | Isg20 | 2.12 |
| 1424208_at | Ptger4 | 2.59 | 1417360_at | Mlh1 | 2.29 | 1448169_at | Krt18 | 2.12 |
| 1419315_at | Slamf9 | 2.59 | 1448211_at | Atp6v0e2 | 2.28 | 1435653_at | Abhd2 | 2.11 |
| 1416414_at | Emilin1 | 2.58 | 1425702_a_at | Enpp5 | 2.28 | 1421025_at | Agpat1 | 2.11 |
| 1448873_at | Ocln | 2.58 | 1434301_at | Fam84b | 2.28 | 1449491_at | Card10 | 2.11 |
| 1437843_s_at | Nupl1 | 2.56 | 1439543_at | 1110064A23Rik | 2.27 | 1454613_at | Dpysl3 | 2.11 |
| 1416110_at | Slc35a4 | 2.56 | 1439451_x_at | Gpr172b | 2.27 | 1422780_at | Pxmp4 | 2.11 |
| 1455065_x_at | Gnpda1 | 2.55 | 1416789_at | Idh3g | 2.27 | 1425148_a_at | Snx6 | 2.11 |
| 1415779_s_at | Actg1 | 2.54 | 1416868_at | Cdkn2c | 2.25 | 1448380_at | Lgals3bp | 2.1 |
| 1454902_at | Prkcz | 2.54 | 1422549_at | Arl2 | 2.24 | 1448569_at | Mlec | 2.1 |
| 1428795_at | 1110021L09Rik | 2.52 | 1431339_a_at | Efhd2 | 2.23 | 1424138_at | Rhbdf1 | 2.1 |
| 1416326_at | Crip1 | 2.52 | 1454606_at | 4933426M11Rik | 2.22 | 1416950_at | Tnfaip8 | 2.1 |
| 1428484_at | Osbpl3 | 2.52 | 1421059_a_at | Alq2 | 2.22 | 1448162_at | Vcam1 | 2.1 |
| 1460732_a_at | Ppl | 2.52 | 1424240_at | Arfip2 | 2.22 | 1428656_at | Rnasen | 2.09 |
| 1438650_x_at | Gja1 | 2.51 | 1423890_x_at | Atp1b1 | 2.22 | 1421843_at | Il1rap | 2.08 |
| 1425173_s_at | Golph3l | 2.5 | 1428442_at | BC029722 | 2.22 | 1433776_at | Lhfp | 2.08 |
| 1437457_a_at | Mtpn | 2.08 | 1452250_a_at | Col6a2 | 1.97 | 1416508_at | Med28 | 1.9 |
| 1429214_at | Adamtsl2 | 2.07 | 1451126_at | Mad | 1.97 | 1456292_a_at | Vim | 1.9 |
| 1451969_s_at | Parp3 | 2.07 | 1418300_a_at | Mknk2 | 1.97 | 1417240_at | Zyx | 1.9 |
| 1451190_a_at | Sbk1 | 2.07 | 1448995_at | Pf4 | 1.97 | 1452304_a_at | Arhgef5 | 1.89 |
| 1416601_a_at | Rcan1 | 2.06 | 1422701_at | Zap70 | 1.97 | 1435758_at | B4galt6 | 1.89 |
| 1448301_s_at | Serpinb1a | 2.06 | 1418819_at | Arl8b | 1.96 | 1448323_a_at | Bgn | 1.89 |
| 1418949_at | Gdf15 | 2.05 | 1419883_s_at | Atp6v1b2 | 1.96 | 1455032_at | Ccnyl1 | 1.89 |
| 1415972_at | Marcks | 2.05 | 1455144_s_at | AU040829 | 1.96 | 1451075_s_at | Ctdsp2 | 1.89 |
| 1421448_at | Ralgapa1 | 2.05 | 1424478_at | Bbs2 | 1.96 | 1416205_at | Glb1 | 1.89 |
| 1427912_at | Cbr3 | 2.04 | 1417176_at | Csnk1e | 1.96 | 1455271_at | Gm13889 | 1.89 |
| 1417837_at | Phlda2 | 2.04 | 1435465_at | Kbtbd11 | 1.96 | 1426523_a_at | Gnpda2 | 1.89 |
| 1436591_at | Vsig10 | 2.04 | 1452046_a_at | Ppp1cc | 1.96 | 1452298_a_at | Myo5b | 1.89 |
| 1422818_at | Nedd9 | 2.03 | 1419493_a_at | Tpd52 | 1.96 | 1426570_a_at | Frk | 1.88 |
| 1449066_a_at | Arhgef7 | 2.02 | 1417848_at | Zfp704 | 1.96 | 1417133_at | Pmp22 | 1.88 |
| 1448823_at | Cxcl12 | 2.02 | 1420965_a_at | End | 1.95 | 1428587_at | Tmem41b | 1.88 |
| 1433870_at | Prr15l | 2.02 | 1436970_a_at | Pdqfrb | 1.95 | 1429722_at | Zbtb4 | 1.88 |
| 1418099_at | Tnfrsf1b | 2.02 | 1418296_at | Fxyd5 | 1.94 | 1419115_at | Alg14 | 1.87 |
| 1450667_a_at | Cs | 2.01 | 1423691_x_at | Krt8 | 1.94 | 1434086_at | Gpr107 | 1.87 |
| 1420394_s_at | Gp49a | 2.01 | 1417324_at | Mast2 | 1.94 | 1426763_at | Oaz2−ps | 1.87 |
| 1429396_at | Atg16l2 | 2 | 1425264_s_at | Mbp | 1.94 | 1451421_a_at | Rogdi | 1.87 |
| 1448405_a_at | Eid1 | 2 | 1450070_s_at | Pak1 | 1.94 | 1415822_at | Scd2 | 1.87 |
| 1424215_at | Fundc1 | 2 | 1434656_at | Ralgapb | 1.94 | 1429089_s_at | 2900026A02Rik | 1.86 |
| 1429461_at | Ints2 | 2 | 1455656_at | Btla | 1.93 | 1455539_at | Gm9983 | 1.86 |
| 1435452_at | Tmem20 | 2 | 1417327_at | Cav2 | 1.93 | 1455750_at | Ralgapa2 | 1.86 |
| 1458347_s_at | Tmprss2 | 2 | 1423392_at | Clic4 | 1.93 | 1418101_a_at | Rtn3 | 1.86 |
| 1436236_x_at | Cotl1 | 1.99 | 1417612_at | Ier5 | 1.93 | 1450138_a_at | Serpinb6a | 1.86 |
| 1426314_at | Ednrb | 1.99 | 1420863_at | Dctn4 | 1.92 | 1417100_at | Cd320 | 1.85 |
| 1449278_at | Eif2ak3 | 1.99 | 1456003_a_at | Slc1a4 | 1.92 | 1451206_s_at | Cytip | 1.85 |
| 1454137_s_at | Hfe2 | 1.99 | 1452081_a_at | 9130017N09Rik | 1.91 | 1449059_a_at | Oxct1 | 1.85 |
| 1450843_a_at | Serpinh1 | 1.99 | 1416455_a_at | Cryab | 1.91 | 1422706_at | Pmepa1 | 1.85 |
| 1455506_at | Slc25a34 | 1.99 | 1450350_a_at | Jdp2 | 1.91 | 1450918_s_at | Src | 1.85 |
| 1424562_a_at | Slc25a4 | 1.99 | 1429527_a_at | Plscr1 | 1.91 | 1450196_s_at | Gys1 | 1.84 |
| 1436729_at | Afap1 | 1.98 | 1436339_at | 1810058I24Rik | 1.9 | 1448606_at | Lpar1 | 1.84 |
| 1460180_at | Hexb | 1.98 | 1432094_a_at | Ccdc132 | 1.9 | 1435548_at | Mrs2 | 1.84 |
| 1416452_at | Oat | 1.98 | 1435223_at | Erlin2 | 1.9 | 1428842_a_at | Ngfrap1 | 1.84 |
| 1433529_at | Pamr1 | 1.98 | 1427474_s_at | Gstm3 | 1.9 | 1428154 S_at | Ppapdc1b | 1.84 |
| 1437234_x_at | Prmt2 | 1.98 | 1453304_s_at | Ly6e | 1.9 | 1449110_at | Rhob | 1.84 |
| 1426434_at | Tmem43 | 1.98 | 1422764_at | Mapre1 | 1.9 | 1439965_at | Slc43a2 | 1.84 |
| 1455308_at | Ano6 | 1.83 | 1434184_s_at | Map4k4 | 1.76 | 1451253_at | Pxk | 1.7 |
| 1436778_at | Cybb | 1.83 | 1418386_at | N6amt2 | 1.76 | 1448204_at | Sav1 | 1.7 |
| 1419505_a_at | Ggps1 1 | .83 | 1454691_at | Nrxn1 | 1.76 | 1452281_at | Sos2 | 1.7 |
| 1416749_at | Htra1 | 1.83 | 1418892_at | Rhoj | 1.76 | 1438289_a_at | Sumo1 | 1.7 |
| 1423584_at | Igfbp7 | 1.83 | 1448568_a_at | Slc20a1 | 1.76 | 1449363_at | Atf3 | 1.69 |
| 1458299_s_at | Nfkbie | 1.83 | 1416627_at | Spint1 | 1.76 | 1416328_a_at | Atp6v0e | 1.69 |
| 1454941_at | Nmt1 | 1.83 | 1416281_at | Wdr45l | 1.76 | 1454636_at | Cbx5 | 1.69 |
| 1448123_s_at | Tgfbi | 1.83 | 1418981_at | Casp12 | 1.75 | 1417104_at | Emp3 | 1.69 |
| 1438246_at | Csnk1g1 | 1.82 | 1417960_at | Cpeb1 | 1.75 | 1429104_at | Limd2 | 1.69 |
| 1434041_at | Appbp2 | 1.81 | 1425747_at | Dock5 | 1.75 | 1427100_at | Metrn | 1.69 |
| 1448669_at | Dkk3 | 1.81 | 1454983_at | Fam63b | 1.75 | 1450971_at | Gadd45b | 1.68 |
| 1460594_a_at | Gmppa | 1.81 | 1438603_x_at | Masp1 | 1.75 | 1448728_a_at | Nfkbiz | 1.68 |
| 1427742_a_at | Klf6 | 1.81 | 1424754_at | Ms4a7 | 1.75 | 1434737_at | Obfc1 | 1.68 |
| 1439364_a_at | Mmp2 | 1.81 | 1449368_at | Dcn | 1.74 | 1436937_at | Rbms3 | 1.68 |
| 1416687_at | Plod2 | 1.81 | 1426648_at | Mapkapk2 | 1.74 | 1431744_a_at | Smap1 | 1.68 |
| 1416230_at | Rfk | 1.81 | 1452067_at | Naaa | 1.74 | 1455513_at | Taf1 | 1.68 |
| 1416009_at | Tspan3 | 1.81 | 1448392_at | Spare | 1.74 | 1423824_at | Wls | 1.68 |
| 1452217_at | Ahnak | 1.8 | 1455566_s_at | Spats2l | 1.74 | 1420008_s_at | Wwc1 | 1.68 |
| 1448901_at | Cpxm1 | 1.8 | 1422751_at | Tle1 | 1.74 | 1434961_at | Asb1 | 1.67 |
| 1424422_s_at | Flad1 | 1.8 | 1430538_at | 2210013O21Rik | 1.73 | 1439902_at | C5ar1 | 1.67 |
| 1424351_at | Wfdc2 | 1.8 | 1460218_at | Cd52 | 1.73 | 1449385_at | Hsd17b6 | 1.67 |
| 1452719_at | Zdhhc24 | 1.8 | 1452359_at | Rell1 | 1.73 | 1437494_at | Mapkapk3 | 1.67 |
| 1437087_at | 2210408K08Rik | 1.79 | 1454890_at | Amot | 1.72 | 1455229_x_at | Pgs1 | 1.67 |
| 1436041_at | LOC100046086 | 1.79 | 1429891_at | Caps I | 1.72 | 1426347_at | 2010321M09Rik | 1.66 |
| 1426728_x_at | Ptdss2 | 1.79 | 1417394_at | Klf4 | 1.72 | 1420820_at | 2900073G15Rik | 1.66 |
| 1435517_x_at | Ralb | 1.79 | 1437165_a_at | Pcolce | 1.72 | 1456307 S_at | Adcy7 | 1.66 |
| 1447621_s_at | Tmem173 | 1.79 | 1434592_at | Slc16a10 | 1.72 | 1451681_at | BC089597 | 1.66 |
| 1440890_a_at | Zfp809 | 1.79 | 1417447_at | Tcf21 | 1.72 | 1419004_s_at | Bcl2a1a | 1.66 |
| 1428373_at | Ip6k2 | 1.78 | 1424829_at | A830007P12Rik | 1.71 | 1420804_s_at | Clec4d | 1.66 |
| 1431056_a_at | Lpl | 1.78 | 1427239_at | Ift122 | 1.71 | 1428861_at | Filip1l | 1.66 |
| 1423989_at | Tecpr1 | 1.78 | 1416590_a_at | Rab34 | 1.71 | 1448396_at | Tmem131 | 1.66 |
| 1452599_s_at | Al413582 | 1.77 | 1434153_at | Shb | 1.71 | 1441811_x_at | Tmem176a | 1.66 |
| 1448682_at | Dynll1 | 1.77 | 1422631_at | Ahr | 1.7 | 1423870_at | Aida | 1.65 |
| 1427537_at | Eppk1 | 1.77 | 1435661_at | Als2cr4 | 1.7 | 1434038_at | Dnajc13 | 1.65 |
| 1418301_at | Irf6 | 1.77 | 1419350_at | Hook2 | 1.7 | 1455793_at | Fam149a | 1.65 |
| 1420477_at | Nap1l1 | 1.77 | 1435514_at | Lztfl1 | 1.7 | 1436243_at | Frmd5 | 1.65 |
| 1460717_at | Tspyl1 | 1.77 | 1451575_a_at | Nudt3 | 1.7 | 1425942_a_at | Gpm6b | 1.65 |
| 1428245_at | G6pc3 | 1.76 | 1426319_at | Pdgfd | 1.7 | 1437716_x_at | Kif22 | 1.65 |
| 1460419_a_at | Prkcb | 1.65 | 1449020_at | Plscr3 | 1.61 | 1425332_at | Zfp106 | 1.57 |
| 1451227_a_at | Slc10a3 | 1.65 | 1417466_at | Rgs5 | 1.61 | 1422013_at | Clec4a2 | 1.56 |
| 1426599_a_at | Slc2a1 | 1.65 | 1415874_at | Spry1 | 1.61 | 1415702_a_at | Ctbp1 | 1.56 |
| 1417635_at | Spa17 | 1.65 | 1434444_s_at | Anapc1 | 1.6 | 1443820_x_at | Elovl1 | 1.56 |
| 1419655_at | Tle3 | 1.65 | 1428549_at | Ccdc3 | 1.6 | 1427927_at | Hscb | 1.56 |
| 1417510_at | Vps4a | 1.65 | 1427301_at | Cd48 | 1.6 | 1421209_s_at | Ikbkg | 1.56 |
| 1453355_at | Wnk2 | 1.65 | 1424113_at | Lamb1−1 | 1.6 | 1424438_a_at | Leprot | 1.56 |
| 1426548_a_at | Atpbd4 | 1.64 | 1421820_a_at | Nf2 | 1.6 | 1422936_at | Mas1 | 1.56 |
| 1426710_at | Calm3 | 1.64 | 1451599_at | Sesn2 | 1.6 | 1456531_x_at | Prpf19 | 1.56 |
| 1422439_a_at | Cdk4 | 1.64 | 1435802_at | Zbtb45 | 1.6 | 1460363_at | Tnrc6c | 1.56 |
| 1433926_at | Dync1li2 | 1.64 | 1436204_at | 1110059G02Rik | 1.59 | 1428945_at | Uba6 | 1.56 |
| 1418049_at | Ltbp3 | 1.64 | 1459962_at | 4930523C07Rik | 1.59 | 1417818_at | Wwtr1 | 1.56 |
| 1436996_x_at | Lyz1 | 1.64 | 1424307_at | Arhgap1 | 1.59 | 1432445_at | 2310016G11Rik | 1.55 |
| 1449498_at | Marco | 1.64 | 1452850_s_at | Brms1l | 1.59 | 1435959_at | Arhgap15 | 1.55 |
| 1421622_a_at | Rapgef4 | 1.64 | 1435910_at | Fads3 | 1.59 | 1419605_at | Clec10a | 1.55 |
| 1452134_at | Tmem175 | 1.64 | 1438562_a_at | Ptpn2 | 1.59 | 1416514_a_at | Fscn1 | 1.55 |
| 1436669_at | 1700019G17Rik | 1.63 | 1424394_at | Selm | 1.59 | 1416554_at | Pdlim1 | 1.55 |
| 1421187_at | Ccr2 | 1.63 | 1427889_at | Spna2 | 1.59 | 1419279_at | Pip4k2a | 1.55 |
| 1428196_a_at | Fam82a2 | 1.63 | 1418744_s_at | Tesc | 1.59 | 1455422_x_at | Sept4 | 1.55 |
| 1423147_at | Mat1a | 1.63 | 1452745_at | Trappc9 | 1.59 | 1455732_at | 1700025G04Rik | 1.54 |
| 1453419_at | Mras | 1.63 | 1420682_at | Chrnb1 | 1.58 | 1428671_at | 2200002D01Rik | 1.54 |
| 1439617_s_at | Pck1 | 1.63 | 1435188_at | Gm129 | 1.58 | 1435751_at | Abcc9 | 1.54 |
| 1428623_at | Plxna1 | 1.63 | 1419193_a_at | Gmfg | 1.58 | 1428103_at | Adam10 | 1.54 |
| 1437832_x_at | Wars | 1.63 | 1417044_at | Lcmt1 | 1.58 | 1422415_at | Ang2 | 1.54 |
| 1419759_at | Abcb1a | 1.62 | 1416808_at | Nid1 | 1.58 | 1448261_at | Cdh1 | 1.54 |
| 1437466_at | Alcam | 1.62 | 1437724_x_at | Pitpnm1 | 1.58 | 1449195_s_at | Cxcl16 | 1.54 |
| 1449870_a_at | Atp6v0a2 | 1.62 | 1422603_at | Rnase4 | 1.58 | 1448557_at | Fam13c | 1.54 |
| 1436921_at | Atp7a s1 | 1.62 | 1450377_at | Thb | 1.58 | 1455002_at | Ptp4a1 | 1.54 |
| 1455688_at | Ddr2 | 1.62 | 1452633_s_at | Aak1 | 1.57 | 1459897_a_at | Sbsn | 1.54 |
| 1452005_at | Dlat | 1.62 | 1451016_at | Ifrd2 | 1.57 | 1449579_at | Sh3yl1 | 1.54 |
| 1428135_a_at | Eef1d | 1.62 | 1423960_at | Lpcat3 | 1.57 | 1422629_s_at | Shroom3 | 1.54 |
| 1428101_at | Rnf38 | 1.62 | 1424850_at | Map3k1 | 1.57 | 1417881_at | Slc39a3 | 1.54 |
| 1416153_at | Srp54a | 1.62 | 1437462_x_at | Mmp15 | 1.57 | 1429556_at | Tead1 | 1.54 |
| 1452150_at | AU040320 | 1.61 | 1417349_at | Pldn | 1.57 | 1453303_at | 4833417J20Rik | 1.53 |
| 1425911_a_at | Fgfr1 | 1.61 | 1423355_at | Snap29 | 1.57 | 1451002_at | Aco2 | 1.53 |
| 1424686_at | Heatr6 | 1.61 | 1427689_a_at | Tnip1 | 1.57 | 1448484_at | Amd1 | 1.53 |
| 1451629_at | Lbh | 1.61 | 1435549_at | Trpm4 | 1.57 | 1434745_at | Ccnd2 | 1.53 |
| 1418231_at | Lims1 | 1.61 | 1456043_at | Usp22 | 1.57 | 1424376_at | Cdc42ep1 | 1.53 |
| 1426955_at | Col18a1 | 1.53 | 1435740_at | Gm10397 | 1.5 | 1423831_at | Prkag2 | −1.54 |
| 1449252_at | Fam110c | 1.53 | 1419197_x_at | Hamp | 1.5 | 1460704_at | Rfng | −1.54 |
| 1421323_a_at | G3bp2 | 1.53 | 1419459_a_at | Magt1 | 1.5 | 1460323_at | Tars | −1.54 |
| 1424994_at | Glyctk | 1.53 | 1429582_at | Nacc2 | 1.5 | 1455278_at | Wdr37 | −1.54 |
| 1426306_a_at | Maged2 | 1.53 | 1429507_at | Nkd1 | 1.5 | 1447550_at | Gm8350 | −1.55 |
| 1422671_s_at | Naalad2 | 1.53 | 1428493_at | Sipa1l3 | 1.5 | 1417932_at | Il18 | −1.55 |
| 1421266_s_at | Nfkbib | 1.53 | 1417392_a_at | Slc7a7 | 1.5 | 1436120_at | Setdb2 | −1.55 |
| 1426726_at | Ppp1r10 | 1.53 | 1430170_at | Bbs10 | −1.5 | 1453065_at | Aldh5a1 | −1.56 |
| 1416360_at | Snx18 | 1.53 | 1442073_at | Inpp1 | −1.5 | 1424583_at | Farp2 | −1.56 |
| 1421891_at | St3qal2 | 1.53 | 1452291_at | Arap2 | −1.51 | 1431078_at | Fbxo3 | −1.56 |
| 1425536_at | Stx3 | 1.53 | 1458295_at | BC038331 | −1.51 | 1418885_a_at | Idh3b | −1.56 |
| 1418004_a_at | Tmem176b | 1.53 | 1424436_at | Gart | −1.51 | 1449157_at | Nr2c1 | −1.56 |
| 1420295_x_at | Clcn5 | 1.52 | 1460689_at | Pppde2 | −1.51 | 1421204_a_at | Nudt16 | −1.56 |
| 1417477_at | Gm16515 | 1.52 | 1422656_at | Rasl2−9−ps | −1.51 | 1438933_x_at | Rasgrp2 | −1.56 |
| 1455277_at | Hhip | 1.52 | 1420919_at | Sgk3 | −1.51 | 1433645_at | Slc44a1 | −1.56 |
| 1448452_at | Irf8 | 1.52 | 1433933_s_at | Slco2b1 | −1.51 | 1436138_at | Ttc19 | −1.56 |
| 1427060_at | Mapk3 | 1.52 | 1443652_x_at | Spred1 | −1.51 | 1416607_at | 4931406C07Rik | −1.57 |
| 1416331_a_at | Nfe2l1 | 1.52 | 1417174_at | Tmem218 | −1.51 | 1428516_a_at | Alkbh7 | −1.57 |
| 1424214_at | Parm1 | 1.52 | 1455281_at | Wdr33 | −1.51 | 1449839_at | Casp3 | −1.57 |
| 1416400_at | Pycrl | 1.52 | 1443901_at | C2cd2 | −1.52 | 1417015_at | Rassf3 | −1.57 |
| 1416882_at | Rgs10 | 1.52 | 1435380_at | Cox10 | −1.52 | 1436167_at | Shf | −1.57 |
| 1434918_at | Sox6 | 1.52 | 1426440_at | Dhrs7 | −1.52 | 1418412_at | Tpd52l1 | −1.57 |
| 1435568_at | Ttc37 | 1.52 | 1437301_a_at | Dvl1 | −1.52 | 1431879_at | 9030417H13Rik | −1.58 |
| 1448100_at | 4833439L19Rik | 1.51 | 1445898_at | Ggcx | −1.52 | 1433759_at | Dpy19l1 | −1.58 |
| 1418128_at | Adcy6 | 1.51 | 1451552_at | Lipt1 | −1.52 | 1437829_s_at | Eef2k | −1.58 |
| 1454169_a_at | Epsti1 | 1.51 | 1418034_at | Mrps9 | −1.52 | 1419228_at | Elac1 | −1.58 |
| 1416199_at | Kifc3 | 1.51 | 1424488_a_at | Ppa2 | −1.52 | 1451058_at | Mcts2 | −1.58 |
| 1455487_at | Mfsd11 | 1.51 | 1424792_at | Rpp40 | −1.52 | 1419400_at | Mttp | −1.58 |
| 1428609_at | Myl12b | 1.51 | 1449125_at | Tnfaip8l1 | −1.52 | 1458408_at | Samd8 | −1.58 |
| 1418831_at | Pkp3 | 1.51 | 1441842_s_at | Zfp707 | −1.52 | 1453208_at | 2700089E24Rik | −1.59 |
| 1416260_a_at | Snx1 | 1.51 | 1447753_at | Cdc37l1 | −1.53 | 1451723_at | Cnot6l | −1.59 |
| 1426248_at | Stk24 | 1.51 | 1450484_a_at | Cmpk2 | −1.53 | 1455163_at | Guf1 | −1.59 |
| 1441945_s_at | Abhd14a | 1.5 | 1417264_at | Coq5 | −1.53 | 1418927_a_at | Habp4 | −1.59 |
| 1450008_a_at | Ctnnb1 | 1.5 | 1451462_a_at | Ifnar2 | −1.53 | 1420846_at | Mrps2 | −1.59 |
| 1426880_at | Etl4 | 1.5 | 1451609_at | Tspan33 | −1.53 | 1416090_at | Pdhb | −1.59 |
| 1443838_x_at | Fads2 | 1.5 | 1438006_at | 4933439F18Rik | −1.54 | 1451956_a_at | Sigmar1 | −1.59 |
| 1423829_at | Fam49b | 1.5 | 1455575_at | Eif4ebp2 | −1.54 | 1437345_a_at | Bscl2 | −1.6 |
| 1421263_at | Gabra3 | 1.5 | 1430555_s_at | Lrig3 | −1.54 | 1451141_at | Mettl8 | −1.6 |
| 1451331_at | Ppp1r1b | −1.6 | 1417434_at | Gpd2 | −1.66 | 1451518_at | Zfp709 | −1.74 |
| 1448800_at | Rtn4ip1 | −1.6 | 1457363_at | LOC654469 | −1.66 | 1432562_at | 1110006G14Rik | −1.75 |
| 1417421_at | S100a1 | −1.6 | 1419173_at | Acy1 | −1.67 | 1418943_at | B230120H23Rik | −1.75 |
| 1418490_at | Sdsl | −1.6 | 1417704_a_at | Arhgap6 | −1.67 | 1433646_at | Mrps27 | −1.75 |
| 1436867_at | Srl | −1.6 | 1419697_at | Cxcl11 | −1.67 | 1421014_a_at | Clybl | −1.76 |
| 1416345_at | Timm8a1 | −1.6 | 1453796_a_at | Ergic2 | −1.67 | 1454867_at | Mn1 | −1.76 |
| 1452626_a_at | 1810014F10Rik | −1.61 | 1428767_at | Gsdmd | −1.67 | 1450852_s_at | F2r | −1.77 |
| 1443873_at | 4933403F05Rik | −1.61 | 1426245_s_at | Mapre2 | −1.67 | 1450869_at | Fgf1 | −1.77 |
| 1419261_at | Acad8 | −1.61 | 1435036_at | Aspg | −1.68 | 1437067_at | Phtf2 | −1.77 |
| 1452532_x_at | Ceacam1 | −1.61 | 1428490_at | C1galt1 | −1.68 | 1430077_at | Sfrs11 | −1.77 |
| 1436532_at | Dclk3 | −1.61 | 1430814_at | Cyp2d40 | −1.68 | 1423447_at | Clpx | −1.79 |
| 1437858_at | Dpy19l3 | −1.61 | 1451426_at | Dhx58 | −1.68 | 1429188_at | Cox11 | −1.79 |
| 1417080_a_at | Ecsit | −1.61 | 1452353_at | Gpr155 | −1.68 | 1458436_at | Auh | −1.8 |
| 1416555_at | Ei24 | −1.61 | 1430287_s_at | Hemk1 | −1.68 | 1425701_a_at | Rgs3 | −1.8 |
| 1424698_s_at | Gca | −1.61 | 1419362_at | Mrpl35 | −1.68 | 1459813_at | 1700012D01Rik | −1.82 |
| 1453678_at | Mbd1 | −1.61 | 1453255_at | Slc43a1 | −1.68 | 1449052_a_at | Dnmt3b | −1.82 |
| 1448825_at | Pdk2 | −1.61 | 1418658_at | Fam82b | −1.69 | 1455037_at | Plxna2 | −1.82 |
| 1459838_s_at | Btbd11 | −1.62 | 1460231_at | Irf5 | −1.69 | 1424022_at | Osgin1 | −1.83 |
| 1437339_s_at | Pcsk5 | −1.62 | 1438640_x_at | Pgk1 | −1.69 | 1449371_at | Hars2 | −1.84 |
| 1452917_at | Rfc5 | −1.62 | 1436058_at | Rsad2 | −1.69 | 1418835_at | Phlda1 | −1.84 |
| 1448930_at | 3010026O09Rik | −1.63 | 1436164_at | Slc30a1 | −1.69 | 1429206_at | Rhobtb1 | −1.84 |
| 1446368_at | 9130221J18Rik | −1.63 | 1452207_at | Cited2 | −1.7 | 1422852_at | Cib2 | −1.85 |
| 1438198_at | Bri3bp | −1.63 | 1428556_at | Pigy | −1.7 | 1418474_at | Fam158a | −1.85 |
| 1455118_at | D9Ertd402e | −1.63 | 1431722_a_at | Afmid | −1.71 | 1448021_at | Fam46c | −1.85 |
| 1432249_a_at | Ercc8 | −1.63 | 1421756_a_at | Gpr19 | −1.71 | 1459860_x_at | Trim2 | −1.85 |
| 1451512_s_at | Hibch | −1.63 | 1428507_at | Hdhd2 | −1.71 | 1431694_a_at | Ctnnbip1 | −1.86 |
| 1435043_at | Plcb1 | −1.63 | 1431591_s_at | Isg15 | −1.71 | 1424352_at | Cyp4a12a | −1.86 |
| 1451277_at | Zadh2 | −1.63 | 1429863_at | Lonrf3 | −1.71 | 1418267_at | Mst1 | −1.86 |
| 1434232_a_at | 2610030H06Rik | −1.64 | 1429216_at | Paqr3 | −1.71 | 1421309_at | Mgmt | −1.87 |
| 1428897_at | 2610029I01Rik | −1.65 | 1420515_a_at | Pglyrp2 | −1.71 | 1424760_a_at | Smyd2 | −1.87 |
| 1451114_at | Cmtm6 | −1.65 | 1437932_a_at | Cldn1 | −1.72 | 1425117_at | Aspdh | −1.88 |
| 1448535_at | Elp4 | −1.65 | 1460591_at | Esr1 | −1.72 | 1427573_at | Chic1 | −1.88 |
| 1423972_at | Etfa | −1.65 | 1449062_at | Khk | −1.72 | 1436959_x_at | Nelf | −1.88 |
| 1451354_at | Foxred1 | −1.65 | 1431032_at | Agl | −1.73 | 1450627_at | Ank | −1.89 |
| 1449348_at | Mpp6 | −1.65 | 1449576_at | Eif1ax | −1.73 | 1426669_at | Cpped1 | −1.89 |
| 1429749_at | Sfmbt1 | −1.65 | 1458678_at | Ndufab1 | −1.73 | 1436070_at | Glo1 | −1.89 |
| 1416479_a_at | Tmem14c | −1.65 | 1416940_at | Ppif | −1.74 | 1431805_a_at | Rhpn2 | −1.89 |
| 1453985_at | 0610007P08Rik | −1.66 | 1443962_at | Tfdp2 | −1.74 | 1431422_a_at | Dusp14 | −1.9 |
| 1437424_at | Syde2 | −1.9 | 1449155_at | Polr3g | −2.21 | 1437424_at | Syde2 | −1.9 |
| 1436109_at | Al317395 | −1.91 | 1422815_at | C9 | −2.24 | 1436109_at | Al317395 | −1.91 |
| 1443822_s_at | Cisd1 | −1.91 | 1453011_at | Bdh2 | −2.25 | 1443822_s_at | Cisd1 | −1.91 |
| 1456767_at | Lrfn3 | −1.91 | 1460059_at | Upp2 | −2.25 | 1456767_at | Lrfn3 | −1.91 |
| 1418997_at | Lyrm5 | −1.91 | 1424692_at | 2810055F11Rik | −2.28 | 1418997_at | Lyrm5 | −1.91 |
| 1420654_a_at | Gbe1 | −1.92 | 1435245_at | Gls2 | −2.28 | 1420654_a_at | Gbe1 | −1.92 |
| 1422399_a_at | Rab23 | −1.93 | 1418311_at | Fn3k | −2.29 | 1422399_a_at | Rab23 | −1.93 |
| 1445787_at | Ccdc162 | −1.94 | 1434692_at | 1110034B05Rik | −2.31 | 1445787_at | Ccdc162 | −1.94 |
| 1442191_at | 5033411D12Rik | −1.95 | 1419510_at | Es22 | −2.32 | 1442191_at | 5033411D12Rik | −1.95 |
| 1448350_at | Asl | −1.95 | 1418645_at | Hal | −2.34 | 1448350_at | Asl | −1.95 |
| 1450033_a_at | Stat1 | −1.95 | 1427213_at | Pfkfb1 | −2.34 | 1450033_a_at | Stat1 | −1.95 |
| 1440688_at | Arhgap26 | −1.96 | 1452975_at | Agxt2l1 | −2.36 | 1440688_at | Arhgap26 | −1.96 |
| 1417869_s_at | Ctsz | −1.97 | 1460318_at | Csrp3 | −2.36 | 1417869_s_at | Ctsz | −1.97 |
| 1456181_at | Wdr91 | −1.98 | 1425778_at | Ido2 | −2.37 | 1456181_at | Wdr91 | −1.98 |
| 1449038_at | Hsd11b1 | −1.99 | 1439459_x_at | Acly | −2.38 | 1449038_at | Hsd11b1 | −1.99 |
| 1452864_at | Med12l | −2.03 | 1429503_at | Fam69a | −2.38 | 1452864_at | Med12l | −2.03 |
| 1428859_at | Paox | −2.03 | 1438055_at | Rarres1 | −2.38 | 1428859_at | Paox | −2.03 |
| 1457027_at | Dhtkd1 | −2.05 | 1429399_at | Rnf125 | −2.39 | 1457027_at | Dhtkd1 | −2.05 |
| 1419670_at | Ftcd | −2.07 | 1449375_at | Ces6 | −2.4 | 1419670_at | Ftcd | −2.07 |
| 1446769_at | Ttc39c | −2.07 | 1453187_at | Ociad2 | −2.4 | 1446769_at | Ttc39c | −2.07 |
| 1441110_at | Lrit1 | −2.08 | 1425778_at | Ido2 | −2.37 | 1451615_at | Ces8 | −2.96 |
| 1428091_at | Klhl7 | −2.09 | 1439459_x_at | Acly | −2.38 | 1422478_a_at | Acss2 | −3.02 |
| 1459141_at | 1810008I18Rik | −2.1 | 1429503_at | Fam69a | −2.38 | 1429642_at | Anubl1 | −3.1 |
| 1424921_at | Bst2 | −2.1 | 1438055_at | Rarres1 | −2.38 | 1424716_at | Retsat | −3.11 |
| 1434410_at | Crybg3 | −2.1 | 1429399_at | Rnf125 | −2.39 | 1451418_a_at | Spsb4 | −3.13 |
| 1450237_at | Dnase2b | −2.11 | 1449375_at | Ces6 | −2.4 | 1453500_at | Cyp2u1 | −3.14 |
| 1418837_at | Qprt | −2.12 | 1453187_at | Ociad2 | −2.4 | 1416795_at | Cryl1 | −3.32 |
| 1430319_at | 4833411C07Rik | −2.13 | 1420603_s_at | Raet1a | −2.44 | 1423186_at | Tiam2 | −3.56 |
| 1449945_at | Ppargc1b | −2.17 | 1422735_at | Foxq1 | −2.45 | 1427052_at | Acacb | −3.57 |
| 1420362_a_at | Bik | −2.19 | 1416049_at | Gldc | −2.46 | 1453752_at | Rpl17 | −3.62 |
| 1437492_at | Mkx | −2.19 | 1421987_at | Papss2 | −2.48 | 1416855_at | Gas1 | −3.73 |
| 1432282_a_at | Tlcd2 | −2.2 | 1418519_at | Aadat | −2.5 | 1421183_at | Tex12 | −3.82 |
| 1433733_a_at | Cry1 | −2.21 | 1427370_at | Amdhd1 | −2.51 | 1417765_a_at | Amy1 | −3.85 |
| 1449155_at | Polr3g | −2.21 | 1438676_at | Mpa2l | −2.55 | 1456074_at | Sdr9c7 | −3.92 |
| 1422815_at | C9 | −2.24 | 1418857_at | Slc13a2 | −2.55 | 1436931_at | Rfx4 | −4.3 |
| 1453011_at | Bdh2 | −2.25 | 1435836_at | Pdk1 | −2.56 | 1421830_at | Ak3 | −4.76 |
| 1460059_at | Upp2 | −2.25 | 1435084_at | C730049O14Rik | −2.57 | 1418780_at | Cyp39a1 | −4.82 |
| 1424692_at | 2810055F11Rik | −2.28 | 1426450_at | Plcl2 | −2.57 | 1453220_at | Fam55b | −5.22 |
| 1435245_at | Gls2 | −2.28 | 1444138_at | Cyp2r1 | −2.6 | 1421092_at | Serpina12 | −5.32 |
| 1418311_at | Fn3k | −2.29 | 1442612_at | C730036E19Rik | −2.65 | 1455383_at | Fam47e | −5.65 |
| 1434692_at | 1110034B05Rik | −2.31 | 1457915_at | 4833442J19Rik | −2.66 | 1450917_at | Myom2 | −5.8 |
| 1419510_at | Es22 | −2.32 | 1454159_a_at | Igfbp2 | −2.66 | 1434449_at | Aqp4 | −6.54 |
| 1418645_at | Hal | −2.34 | 1448898_at | Ccl9 | −2.78 | 1455991_at | Ccbl2 | −7.41 |
| 1427213_at | Pfkfb1 | −2.34 | 1437250_at | Mreg | −2.78 | 1420722_at | Elovl3 | −9.89 |
| 1452975_at | Aqxt2l1 | −2.36 | 1417828_at | Aqp8 | −2.92 | 1423397_at | Ugt2b38 | −22.52 |
| 1460318_at | Csrp3 | −2.36 | 1457619_at | BC015286 | −2.92 | | | |

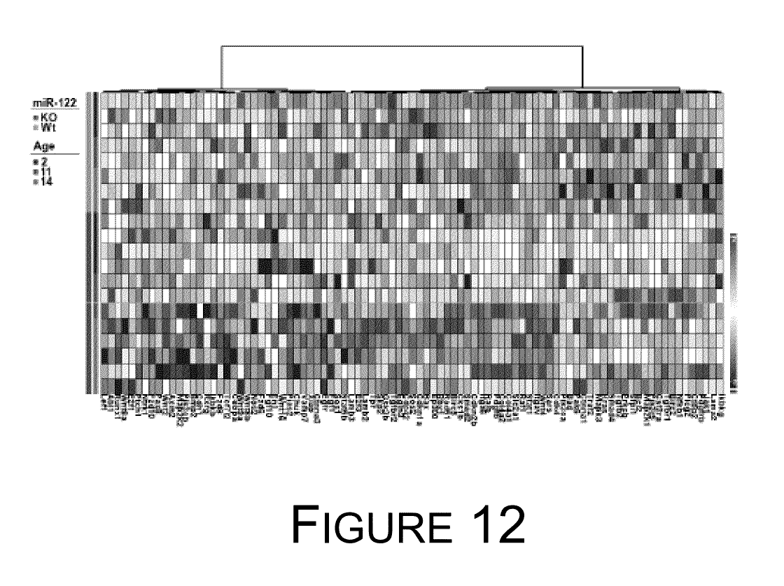

Mir-122 deficiency appeared to create a permissive microenviroment for fibrotic activity and for hepatocyte proliferation, which was explicitly illustrated from the expression patterns of the genes for fibrosis and proliferation in the KEGG “pathways in cancer” (FIG. 10b, FIG. 12, Supplementary Table 4).

SUPPLEMENTARY TABLE 4
Relative expression levels of genes in KEGG “Pathway in cancer” gene set.

| | 2 month KO/WT | | 11−month KO−T/WT | | 14−month KO−T/WT | |
|---|---|---|---|---|---|---|
| Gene Symbol | Fold−Change | p−value * | Fold−Change | p-value * | Fold-Change | p-value * |
| Lef1 | −1.00 | 0.9937 | 1.04 | 0.9588 | −3.81 | 0.0368 |
| Abl1 | −1.42 | 0.5970 | −1.60 | 0.4978 | −2.23 | 0.0130 |
| Runx1 | 1.19 | 0.7158 | −1.45 | 0.2121 | −3.51 | 0.0035 |
| Wnt8a | 1.25 | 0.6128 | −1.01 | 0.9749 | −3.60 | 0.0467 |
| E2f1 | 1.36 | 0.6599 | −1.42 | 0.4926 | −3.39 | 0.0093 |
| Ptch1 | 1.11 | 0.7595 | −1.21 | 0.4086 | −5.15 | 0.0049 |
| Ntrk1 | 1.57 | 0.3515 | −1.62 | 0.5286 | −2.21 | 0.0297 |
| Fzd10 | −1.22 | 0.5763 | −1.98 | 0.2710 | −2.96 | 0.0072 |
| Pax8 | −1.20 | 0.7203 | −2.77 | 0.0951 | −2.73 | 0.0137 |
| Wnt2 | −1.10 | 0.5226 | −1.83 | 0.2022 | −2.57 | 0.0242 |
| Axin2 | −1.01 | 0.9730 | −3.13 | 0.0124 | −2.07 | 0.0102 |
| Map2k2 | 1.73 | 0.0925 | −2.19 | 0.2343 | −2.85 | 0.0177 |
| Pik3cb | 1.02 | 0.9300 | −2.50 | 0.2774 | −3.13 | 0.0149 |
| Bmp2 | −1.06 | 0.7104 | −3.01 | 0.2295 | −3.72 | 0.0078 |
| Cdh1 | 1.42 | 0.0606 | −1.35 | 0.5878 | −2.50 | 0.0136 |
| Rxrg | −1.30 | 0.6610 | −2.75 | 0.3143 | −2.15 | 0.0363 |
| Ikbkb | 1.12 | 0.8191 | −2.06 | 0.4340 | −3.13 | 0.0111 |
| Fzd8 | −1.54 | 0.1649 | −1.84 | 0.4194 | −5.33 | 0.0010 |
| Tcf7l2 | −1.50 | 0.1524 | −2.15 | 0.1903 | −3.69 | 0.0350 |
| Cebpa | 1.00 | 0.9983 | −2.74 | 0.2044 | −3.72 | 0.0107 |
| Wnt9a | −1.18 | 0.7724 | −1.44 | 0.4010 | −3.00 | 0.0327 |
| Wnt8b | −2.18 | 0.0949 | −2.17 | 0.4091 | −3.09 | 0.0089 |
| Nos2 | 1.32 | 0.3145 | 3.19 | 0.1914 | −3.52 | 0.0040 |
| Fzd6 | −2.25 | 0.2104 | 1.01 | 0.9751 | −3.87 | 0.0091 |
| Fgf10 | −1.98 | 0.4092 | 1.20 | 0.7459 | −2.28 | 0.0329 |
| Fn1 | −2.92 | 0.1627 | −1.80 | 0.0002 | −2.58 | 0.0096 |
| Wnt16 | −2.26 | 0.2372 | −1.14 | 0.7458 | −2.20 | 0.0293 |
| Pias2 | −1.34 | 0.4743 | −2.34 | 0.2887 | −3.17 | 0.0428 |
| Chuk | −1.67 | 0.3334 | −2.23 | 0.3575 | −2.91 | 0.0059 |
| Vamp7 | −2.01 | 0.2909 | −2.69 | 0.0910 | −3.66 | 0.0001 |
| Xiap | −1.70 | 0.3730 | −2.82 | 0.1210 | −3.47 | 0.0007 |
| Ctnna3 | −1.43 | 0.3727 | −3.23 | 0.0195 | −3.33 | 0.0008 |
| Egfr | −1.47 | 0.0250 | −2.98 | 0.0534 | −4.18 | 0.0002 |
| Fgf1 | −1.95 | 0.0061 | −2.43 | 0.0002 | −4.65 | 0.0029 |
| Sos1 | −1.53 | 0.2948 | −4.74 | 0.0483 | −2.71 | 0.0213 |
| Stat5b | −2.66 | 0.0014 | −2.69 | 0.1896 | −2.79 | 0.0303 |
| Rad51 | 1.13 | 0.4269 | 1.56 | 0.5724 | 3.40 | 0.0111 |
| Birc5 | 1.26 | 0.1015 | 1.33 | 0.7433 | 3.43 | 0.0044 |
| Cks1b | 1.57 | 0.1427 | 1.38 | 0.6784 | 3.53 | 0.0393 |
| Smad2 | 1.21 | 0.7087 | −1.06 | 0.9574 | 2.97 | 0.0069 |
| Lamb3 | −1.41 | 0.2774 | 2.85 | 0.2053 | 2.27 | 0.0363 |
| E2f3 | −1.85 | 0.1112 | −1.15 | 0.8103 | 3.17 | 0.0488 |
| Lamb2 | 1.31 | 0.1156 | 1.69 | 0.1405 | 3.34 | 0.0017 |
| Tpr | 1.04 | 0.8971 | 1.91 | 0.0855 | 3.86 | 0.0003 |
| Skp2 | −1.08 | 0.7968 | −1.04 | 0.9393 | 3.25 | 0.0348 |
| Gsk3b | 1.29 | 0.2899 | 1.66 | 0.2111 | 3.46 | 0.0085 |
| Tgfbr2 | 1.67 | 0.0814 | 2.13 | 0.0910 | 3.80 | 0.0003 |
| Egln3 | 1.34 | 0.0975 | 3.09 | 0.0737 | 5.08 | 0.0001 |
| Cdc42 | 1.36 | 0.1314 | 2.60 | 0.1104 | 4.08 | 0.0019 |
| Sos2 | 1.87 | 0.2385 | 1.93 | 0.0554 | 3.21 | 0.0178 |
| Cdkn1a | 1.07 | 0.8888 | 2.90 | 0.0756 | 3.45 | 0.0068 |
| Bax | 1.59 | 0.2257 | 2.08 | 0.3080 | 3.64 | 0.0030 |
| Ep300 | 2.59 | 0.0016 | 1.76 | 0.1859 | 4.10 | 0.0032 |
| Rbx1 | 1.60 | 0.0217 | 2.21 | 0.0874 | 3.93 | 0.0076 |
| Cdkn2b | 1.54 | 0.1881 | 2.02 | 0.2487 | 4.42 | 0.0069 |
| Itga6 | 1.53 | 0.0961 | 2.70 | 0.1392 | 4.41 | 0.0018 |
| Ralb | 1.78 | 0.0014 | 3.19 | 0.0192 | 4.73 | 0.0002 |
| Pdgfb | 1.27 | 0.0207 | 3.22 | 0.1492 | 3.65 | 0.0173 |
| Col4a2 | 1.55 | 0.0520 | 3.63 | 0.0987 | 4.24 | 0.0029 |
| Col4a1 | 1.51 | 0.0460 | 4.07 | 0.0308 | 4.85 | 0.0007 |
| Slc2a1 | 1.46 | 0.0392 | 2.24 | 0.2130 | 4.10 | 0.0117 |
| Raf1 | 1.23 | 0.1339 | 1.78 | 0.4081 | 3.34 | 0.0327 |
| Stat1 | −1.58 | 0.0055 | 2.00 | 0.3618 | 3.66 | 0.0042 |
| Itgav | 1.06 | 0.5898 | 2.93 | 0.1185 | 3.73 | 0.0013 |
| Wnt4 | 1.07 | 0.4217 | 2.12 | 0.2401 | 3.77 | 0.0299 |
| Sars | 1.73 | 0.0028 | 3.11 | 0.0776 | 3.66 | 0.0317 |
| Cdk4 | 1.81 | 0.0377 | 3.29 | 0.1271 | 3.52 | 0.0255 |
| Pik3ca | 1.09 | 0.8721 | 3.22 | 0.1558 | 2.56 | 0.0370 |
| Bad | 2.50 | 0.2610 | 1.75 | 0.2699 | 2.03 | 0.0294 |
| Pak6 | 1.72 | 0.0343 | 2.40 | 0.0461 | 4.82 | 0.0062 |
| Ctnna1 | 1.77 | 0.0946 | 2.48 | 0.1021 | 4.33 | 0.0012 |
| Traf2 | 1.55 | 0.1852 | 3.05 | 0.0029 | 5.03 | 0.0024 |
| Mapk3 | 2.66 | 0.0225 | 4.07 | 0.0105 | 3.83 | 0.0009 |
| Kras | 2.09 | 0.0249 | 4.19 | 0.0348 | 4.18 | 0.0006 |
| Smad4 | 1.69 | 0.0652 | 1.45 | 0.5585 | 5.82 | 0.0010 |
| Tgfb2 | 2.20 | 0.0596 | 4.42 | 0.0611 | 2.61 | 0.0318 |
| Prkcb | 1.71 | 0.1263 | 4.98 | 0.1204 | 2.41 | 0.0338 |
| Sfpi1 | 1.64 | 0.2466 | 4.66 | 0.0804 | 2.77 | 0.0127 |
| Bcl2 | 2.19 | 0.1253 | 2.65 | 0.1448 | 3.08 | 0.0108 |
| Map2k1 | 1.50 | 0.3769 | 2.37 | 0.2503 | 4.08 | 0.0035 |
| Pik3r5 | 2.43 | 0.0132 | 5.22 | 0.0743 | 2.43 | 0.0017 |
| Csf2ra | 1.60 | 0.0053 | 4.65 | 0.0551 | 4.09 | 0.0001 |
| Tgfbr1 | 1.44 | 0.1596 | 3.34 | 0.1043 | 4.18 | 0.0002 |
| Birc2 | 2.18 | 0.0051 | 2.06 | 0.4512 | 2.99 | 0.0012 |
| Nfkb1 | 1.28 | 0.2917 | 4.55 | 0.0759 | 3.19 | 0.0116 |
| Plcg2 | 2.02 | 0.0247 | 5.02 | 0.1473 | 2.26 | 0.0314 |
| Ctbp2 | 2.57 | 0.0032 | 3.70 | 0.1341 | 3.35 | 0.0060 |
| Pdgfrb | 2.65 | 0.0005 | 3.59 | 0.0507 | 3.28 | 0.0353 |
| Jak1 | 2.48 | 0.0020 | 2.88 | 0.1146 | 3.99 | 0.0100 |
| Lama2 | 3.31 | 0.0119 | 2.02 | 0.2691 | 2.43 | 0.0217 |
| Ikbkg | 2.55 | 0.0267 | 1.12 | 0.8856 | 3.10 | 0.0133 |

* p-value determined by unpaired, two-tailed Student's t-test.
2 month KO/WT: expression fold-change of 122KO and Wild-type livers.
11-month KO-T/WT: expression fold-change of tumors from 122KO livers and WT livers.
14-month KO-T/WT: expression fold-change of tumors from 122KO livers and WT livers

### Example 7

### mir-122 Target Genes in the Liver

We next investigated how the large repertoire of mir-122's target genes that are dynamically present over the entire life span contributed to the control of mir-122 in the liver. We predicted 252 human-mouse orthologs as potential mir-122 target genes (Supplementary Table 5).

SUPPLEMENTARY TABLE 5
Nucleotide positions of the predicted mir-122-binding sites within the 3′UTR of the candidate target genes.

| | # Binding | | |
|---|---|---|---|
| Genes | sites | Sc-M of each binding site | 3′UTR locations ^{¶} of the predicted binding site |
| 1110021L09Rik | 4 | 122.00, 138.00, 120.00, 147.00 | 294-318, 528-549, 585-612, 1162-1186 |
| 1700025G04Rik | 2 | 158.00, 126.00 | 6633-6657, 8607-8632 |
| 4933426M11Rik | 4 | 125.00, 120.00, 131.00, 140.00 | 1114-1147, 1684-1705, 2618-2642, 2808-2837 |
| AA986860 | 2 | 120.00, 153.00 | 660-681, 696-718 |
| Aak1 | 8 | 120.00, 142.00, 130.00, 121.00, 129.00, | 340-361, 3927-3952, 3989-40105063-5090, 5397- |
| | | 127.00, 124.00, 128.00 | 5420, 6070-6092, 9708-9730, 12757-12781 |
| Abcc9 | 4 | 137.00, 142.00, 132.00, 138.00 | 447-471, 620-642, 704-731, 1155-1176 |
| Abhd2 | 1 | 136 | 779-800 |
| Adam10 | 1 | 120 | 36-57 |
| Adamtsl2 | 1 | 128 | 151-170 |
| Adcy6 | 1 | 134 | 2191-2212 |
| Agpat1 | 6 | 120.00, 123.00, 120.00, 130.00, 151.00, | 63-84, 111-136, 165-186, 247-272, 441-458, 554- |
| | | 131.00 | 580 |
| Ahr | 1 | 124 | 35-57 |
| Aldoa | 1 | 148 | 17-40 |
| Alpl | 3 | 140.00, 140.00, 153.00 | 295-316, 480-501, 509-529 |
| Amot | 1 | 131 | 2444-2463 |
| Ankrd13c | 3 | 142.00, 145.00, 139.00 | 227-251, 279-304, 504-530 |
| Ano6 | 4 | 153.00, 131.00, 127.00, 120.00 | 514-540, 684-705, 1783-1803, 1884-1905 |
| Arfip2 | 2 | 121.00, 151.00 | 233-260,264-310 |
| Arhgap1 | 3 | 122.00, 120.00, 157.00 | 521-552, 584-605, 1231-1252 |
| Arl2 | 1 | 152 | 56-85 |
| Arl8b | 1 | 133 | 1375-1402 |
| Asb1 | 3 | 136.00, 148.00, 148.00 | 171-191, 771-800, 3554-3571 |
| Atp11a | 6 | 140.00, 150.00, 135.00, 130.00, | 672-691, 960-985, 1831-1852, 1953-1979, 2018- |
| | | 156.00, 128.00 | 2046, 3499-3524 |
| Atp1b1 | 1 | 164 | 518-540 |
| Atp6v0a2 | 2 | 123.00, 121.00 | 36-75, 164-191 |
| Atp6v0e2 | 4 | 123.00, 144.00, 120.00, 123.00 | 292-309, 544-569, 695-716, 726-746 |
| Atp7a | 2 | 120.00, 128.00 | 1382-1403, 2750-2776 |
| Atpbd4 | 4 | 148.00, 139.00, 146.00, 160.00 | 228-248, 403-425, 1347-1373, 1402-1420 |
| Atpif1 | 1 | 120 | 25-46 |
| AU040320 | 3 | 120.00, 125.00, 124.00 | 101-122, 269-291, 601-623 |
| AU040829 | 1 | 136 | 80-102 |
| B230208H17Rik | 3 | 134.00, 124.00, 157.00 | 317-333, 664-687, 974-997 |
| Bcat2 | 1 | 151 | 126-155 |
| Btla | 1 | 146 | 1894-1930 |
| Card10 | 1 | 126 | 1165-1191 |
| Cav2 | 2 | 127.00, 135.00 | 421-441, 891-917 |
| CbxS | 4 | 135.00, 123.00, 153.00, 151.00 | 2682-2707, 4324-4349, 7203-7221, 7529-7554 |
| Ccdc3 | 3 | 127.00, 121.00, 124.00 | 1183-1205, 1425-1455, 1539-1560 |
| Ccnd1 | 1 | 120 | 514-535 |
| Ccnd2 | 3 | 136.00, 156.00, 126.00 | 2939-2966, 2987-3008, 3134-3152 |
| Ccnyl1 | 3 | 120.00, 153.00, 124.00 | 209-230, 630-653, 1060-1083 |
| Ccr2 | 1 | 160 | 856-885 |
| Ccrn4l | 2 | 120.00, 140.00 | 67-103, 147-178 |
| Cd320 | 2 | 148.00, 154.00 | 154-181, 260-284 |
| Cda | 2 | 130.00, 129.00 | 137-160, 206-237 |
| Cdc42ep1 | 1 | 128 | 227-261 |
| Cdh1 | 1 | 127 | 116-143 |
| Cldn2 | 2 | 126.00, 120.00 | 1224-1262, 1703-1724 |
| Cldn7 | 1 | 120 | 37-60 |
| Clic1 | 1 | 163 | 69-92 |
| Clic4 | 3 | 154.00, 126.00, 143.00 | 138-162, 1560-1593, 2629-2655 |
| Clic5 | 2 | 124.00, 155.00 | 2037-2065, 4569-4594 |
| Col3a1 | 1 | 143 | 273-293 |
| Cpeb1 | 2 | 144.00, 156.00 | 356-377, 581-602 |
| Cpne8 | 1 | 126 | 10-37 |
| Crispld2 | 5 | 152.00, 144.00, 134.00, 140.00, 120.00 | 276-297, 518-539, 586-608, 905-924,1741-1762 |
| Cs | 1 | 146 | 1150-1173 |
| Csnk1g1 | 6 | 134.00, 128.00, 121.00, 131.00, 122.00, | 1647-1675, 2663-2690, 4250-4273, 4301-4321, |
| | | 144.00 | 4555-4573, 4839-4871 |
| Ctps2 | 2 | 140.00, 121.00 | 792-815, 1327-1349 |
| Cxcl12 | 4 | 145.00, 125.00, 135.00, 123.00 | 166-187, 886-914, 1722-1746, 4167-4186 |
| Cybb | 1 | 130 | 946-969 |
| Cygb | 1 | 147 | 224-246 |
| Ddit4l | 1 | 140 | 1091-1119 |
| Ddr1 | 1 | 151 | 155-175 |
| Ddr2 | 1 | 120 | 65-86 |
| Dlat | 2 | 154.00, 145.00 | 184-212, 1397-1424 |
| Dock5 | 4 | 154.00, 144.00, 138.00, 129.00 | 405-424, 830-852, 3384-3406, 4358-4382 |
| Dpt | 2 | 135.00, 133.00 | 86-102, 200-224 |
| Dynll1 | 1 | 132 | 727-750 |
| Elovl1 | 1 | 127 | 188-209 |
| Emilin1 | 1 | 120 | 112-137 |
| Emp1 | 1 | 128 | 1070-1094 |
| Enc1 | 2 | 139.00, 134.00 | 148-169, 1303-1324 |
| Endod1 | 2 | 155.00, 122.00 | 1847-1870, 2277-2306 |
| Enpp5 | 2 | 155.00, 124.00 | 153-179, 402-426 |
| Erlin2 | 2 | 122.00, 161.00 | 77-98, 636-653 |
| Ezr | 2 | 127.00, 134.00 | 28-61, 794-820 |
| Fam149a | 2 | 139.00, 123.00 | 348-386, 1308-1330 |
| Fam49b | 2 | 123.00, 148.00 | 1858-1879, 2260-2282 |
| Fam82a2 | 1 | 136 | 514-540 |
| Fbln5 | 2 | 139.00, 157.00 | 3088-3112, 3474-3508 |
| Fgfr1 | 1 | 127 | 1194-1215 |
| Flnb | 3 | 124.00, 135.00, 140.00 | 112-134, 421-441, 503-535 |
| Fmo2 | 2 | 123.00, 158.00 | 539-566, 1490-1516 |
| Fuca2 | 2 | 147.00, 126.00 | 299-317, 1465-1486 |
| G3bp2 | 4 | 137.00, 145.00, 127.00, 124.00 | 649-671, 806-827, 1229-1250, 1642-1662 |
| G6pc3 | 1 | 151 | 18-62 |
| Gabra3 | 1 | 135 | 1344-1367 |
| Gde1 | 2 | 128.00, 120.00 | 50-71, 286-323 |
| Gfpt1 | 3 | 136.00, 123.00, 154.00 | 369-391, 680-706, 1874-1912 |
| Ggps1 | 1 | 148 | 114-140 |
| Gja1 | 2 | 124.00, 144.00 | 466-487, 1585-1611 |
| Glyctk | 1 | 128 | 780-830 |
| Gmppa | 1 | 145 | 24-45 |
| Gnpda1 | 3 | 130.00, 161.00, 130.00 | 487-514, 780-802, 955-971 |
| Gnpda2 | 2 | 142.00, 146.00 | 143-171, 332-361 |
| Golph3l | 5 | 128.00, 135.00, 151.00, 154.00, 131.00 | 92-120, 399-431, 532-553, 583-608, 1063-1083 |
| Gpm6b | 1 | 172 | 54-74 |
| Gpr107 | 1 | 150 | 77-110 |
| Gpr172b | 1 | 163 | 109-132 |
| Gys1 | 3 | 120.00,154.00,136.00 | 70-97, 131-154, 160-191 |
| Hhip | 10 | 127.00, 130.00, 132.00, 141.00, 145.00, | 460-480, 908-926, 1157-1179, 1372-1398, 1844- |
| | | 138.00, 122.00, 120.00, 138.00, 120.00 | 1860, 2937-2969, 3423-3454, 3476-3501, 3659- |
| | | | 3684, 5554-5591 |
| Idh3a | 4 | 120.00, 154.00, 120.00, 157.00 | 22-46, 274-293, 700-721, 783-803 |
| Idh3g | 1 | 136 | 41-63 |
| Igf2 | 4 | 120.00, 140.00, 129.00, 120.00 | 90-111, 930-955, 2115-2137, 2298-2319 |
| Igfbp7 | 1 | 138 | 65-86 |
| Ikbkg | 8 | 124.00, 143.00, 159.00, 124.00, 121.00, | 65-91, 776-796,943-968,1038-1059,1748-1763, |
| | | 120.00, 159.00, 127.00 | 1869-1890, 4228-4259, 5338-5359 |
| Ints2 | 1 | 127 | 1343-1362 |
| Itgb8 | 1 | 128 | 269-317 |
| Jdp2 | 2 | 128.00, 128.00 | 236-260, 308-325 |
| Jun | 1 | 140 | 860-883 |
| Kbtbd11 | 2 | 123.00, 130.00 | 75-96, 4529-4564 |
| Kctd17 | 1 | 147 | 162-183 |
| Klf6 | 1 | 158 | 953-976 |
| Lars2 | 1 | 161 | 586-610 |
| Lass6 | 3 | 124.00, 120.00, 148.00 | 281-303, 1498-1519, 2004-2024 |
| Lcmt1 | 1 | 122 | 180-204 |
| Leprot | 2 | 122.00, 120.00 | 154-178, 324-346 |
| Lhfp | 1 | 136 | 406-427 |
| Limd2 | 1 | 135 | 811-843 |
| Lpar1 | 1 | 140 | 76-115 |
| Lpcat3 | 2 | 138.00, 128.00 | 52-76, 87-119 |
| Lpl | 1 | 155 | 1460-1484 |
| Lztfl1 | 4 | 133.00, 143.00, 136.00, 140.00 | 208-235, 1048-1071, 1148-1169, 2054-2075 |
| Maf1 | 1 | 148 | 316-334 |
| Maged2 | 2 | 142.00, 138.00 | 287-312, 324-348 |
| Map3k1 | 1 | 139 | 1577-1603 |
| Map4k4 | 1 | 127 | 1472-1491 |
| Mapk3 | 2 | 132.00, 139.00 | 163-189, 391-421 |
| Mapkapk2 | 2 | 124.00, 120.00 | 270-297, 879-907 |
| Mapre1 | 5 | 132.00, 135.00, 140.00, 140.00, 134.00 | 61-84, 90-112, 874-895, 2248-2270, 3273-3294 |
| Marcks | 1 | 132 | 3501-3526 |
| Mast2 | 1 | 128 | 22-48 |
| Mcrs1 | 1 | 149 | 226-255 |
| Med28 | 3 | 127.00, 131.00, 150.00 | 915-950, 2154-2170, 3917-3936 |
| Mfge8 | 1 | 145 | 383-400 |
| Mfsd11 | 4 | 120.00, 140.00, 132.00, 127.00 | 337-358, 496-520, 679-697, 884-906 |
| Mknk2 | 1 | 120 | 302-323 |
| Mlec | 3 | 143.00, 120.00, 120.00 | 365-384, 2426-2447, 4843-4864 |
| Mmp2 | 1 | 124 | 503-524 |
| Mras | 4 | 130.00, 133.00, 120.00, 144.00 | 506-529, 1199-1219, 2146-2173, 2182-2205 |
| Mrs2 | 1 | 145 | 376-397 |
| Mtmr11 | 1 | 122 | 69-93 |
| Mtpn | 1 | 131 | 576-594 |
| Myo5b | 1 | 144 | 986-1008 |
| Naaa | 1 | 160 | 252-273 |
| Nap1l1 | 3 | 132.00, 142.00, 120.00 | 1241-1267, 1521-1552, 1857-1882 |
| Ndrg3 | 3 | 120.00, 151.00, 156.00 | 36-57, 182-207, 1251-1278 |
| Necap2 | 3 | 131.00, 141.00, 135.00 | 150-182, 196-218, 387-406 |
| Nedd9 | 1 | 148 | 1571-1593 |
| Nf2 | 4 | 122.00, 144.00, 158.00, 123.00 | 396-424, 729-750, 999-1025, 2271-2290 |
| Nfe2l1 | 1 | 124 | 737-762 |
| Nfkbiz | 1 | 120 | 1276-1303 |
| Nid1 | 2 | 147.00, 120.00 | 31-52, 953-974 |
| Nkd1 | 3 | 123.00, 132.00, 154.00 | 446-467, 871-892, 1179-1203 |
| Nrxn1 | 3 | 132.00, 129.00, 140.00 | 3219-3257, 3292-3317, 3370-3392 |
| Ntrk2 | 6 | 120.00, 135.00, 132.00, 121.00, 145.00, | 1317-1338, 1541-1559, 2061-2086, 3122-3145, |
| | | 134.00 | 3960-3987, 4483-4504 |
| Nupl1 | 1 | 135 | 102-129 |
| Obfc1 | 1 | 165 | 319-348 |
| Ocln | 2 | 143.00, 120.00 | 191-220, 332-372 |
| Oxct1 | 3 | 129.00, 120.00, 120.00 | 450-471, 1006-1035, 1173-1194 |
| P4ha1 | 3 | 149.00, 152.00, 130.00 | 74-95, 1768-1790, 2187-2219 |
| Parp3 | 1 | 129 | 254-282 |
| Pdgfra | 1 | 122 | 2844-2894 |
| Pdgfrb | 2 | 120.00, 144.00 | 615-636, 985-1012 |
| Pfkfb3 | 2 | 120.00, 126.00 | 1058-1079, 2263-2290 |
| Pip4k2a | 2 | 139.00, 151.00 | 218-242, 534-556 |
| Pldn | 3 | 120.00, 133.00, 134.00 | 695-720, 1376-1396, 1522-1550 |
| Plp2 | 1 | 148 | 177-202 |
| Plscr1 | 1 | 123 | 137-165 |
| Plscr3 | 4 | 167.00, 136.00, 144.00, 150.00 | 259-279, 1044-1071, 1445-1465, 1471-1490 |
| Plxna1 | 4 | 120.00, 150.00, 130.00, 151.00 | 573-594, 1544-1577, 2400-2429, 2827-2847 |
| Pnpla6 | 1 | 148 | 64-103 |
| Ppapdc1b | 2 | 121.00, 136.00 | 241-283, 385-406 |
| Ppl | 1 | 124 | 768-808 |
| Ppp1cc | 1 | 157 | 535-559 |
| Ppp1r10 | 2 | 136.00, 123.00 | 199-219, 665-686 |
| Prkcb | 5 | 136.00, 132.00, 133.00, 132.00, 127.00 | 1484-1506, 1508-1533, 3001-3020, 3826-3843, |
| | | | 4858-4878 |
| Prlr | 12 | 134.00, 148.00, 128.00, 134.00, 143.00, | 149-167, 489-507, 538-564, 1675-1698, 2514-2534, |
| | | 133.00, 130.00, 120.00, 138.00, 159.00, | 2850-2866, 2941-2967, 3375-3395, 5462-5483, |
| | | 120.00, 127.00 | 5749-5773, 6798-6826, 7242-7265 |
| Ptdss2 | 1 | 147 | 99-118 |
| Pxmp4 | 1 | 162 | 125-150 |
| Pycrl | 1 | 151 | 345-373 |
| Rad51l1 | 1 | 127 | 619-651 |
| Ralb | 2 | 144.00, 149.00 | 1109-1137, 1186-1202 |
| Rbms3 | 1 | 148 | 5360-5385 |
| Rcan1 | 1 | 128 | 581-602 |
| Rell1 | 2 | 137.00, 142.00 | 264-285, 736-753 |
| Rhob | 1 | 132 | 913-937 |
| Rnf38 | 6 | 130.00, 140.00, 132.00, 160.00, 153.00, | 234-261, 646-668, 773-794, 1203-1225, 2919-2943, |
| | | 136.00 | 3359-3380 |
| Rogdi | 1 | 146 | 225-255 |
| Rtn3 | 1 | 158 | 1571-1595 |
| Sav1 | 1 | 124 | 1029-1050 |
| Sbk1 | 6 | 144.00, 136.00, 125.00, 156.00, 122.00, | 238-256, 613-635, 673-693, 855-887, 1178-1205, |
| | | 125.00 | 2213-2242 |
| Scara3 | 2 | 121.00, 148.00 | 796-819, 1090-1135 |
| Serpine2 | 1 | 132 | 392-413 |
| Serpinh1 | 2 | 120.00, 122.00 | 158-181, 394-415 |
| Sesn2 | 1 | 134 | 409-434 |
| Shb | 1 | 131 | 549-569 |
| Slc10a3 | 1 | 131 | 24-46 |
| Slc1a4 | 6 | 124.00, 124.00, 138.00, 141.00, 138.00, | 159-180, 627-650, 812-835, 1105- |
| | | 130.00 | 1129, 1184-1208, 1448-1468 |
| Slc25a34 | 3 | 160.00, 139.00, 159.00 | 2010-2031, 2040-2062, 2187-2207 |
| Slc2a1 | 2 | 122.00, 143.00 | 268-287, 709-727 |
| Slc35a4 | 4 | 139.00, 125.00, 141.00, 124.00 | 198-218, 328-347, 518-539, 598-619 |
| Slc39a3 | 2 | 152.00, 139.00 | 2075-2098, 2285-2330 |
| Slc43a2 | 1 | 140 | 451-477 |
| Slc5a1 | 2 | 146.00, 130.00 | 183-206, 983-1007 |
| Slc6a8 | 3 | 134.00, 122.00, 132.00 | 686-712, 883-909, 1281-1309 |
| Slc7a7 | 1 | 126 | 221-249 |
| Smap1 | 3 | 131.00, 152.00, 123.00 | 94-110, 262-284, 537-554 |
| Snap29 | 4 | 126.00, 123.00, 147.00, 120.00 | 349-371, 903-937, 1326-1347, 2098-2119 |
| Snx18 | 2 | 126.00, 132.00 | 415-436, 834-859 |
| Snx6 | 1 | 140 | 178-201 |
| Sox4 | 1 | 120 | 242-274 |
| Sox6 | 5 | 130.00, 120.00, 120.00, 137.00, 147.00 | 14-35, 2665-2701, 2848-2872, 3695-3721, 5512- |
| | | | 5534 |
| Sparc | 1 | 131 | 571-592 |
| Sptlc2 | 3 | 133.00, 122.00, 144.00 | 93-115, 158-174, 1668-1695 |
| Src | 1 | 129 | 1780-1801 |
| Src | 1 | 129 | 1780-1801 |
| St3gal2 | 2 | 136.00, 121.00 | 342-364, 485-512 |
| Stk24 | 1 | 149 | 647-667 |
| Stx3 | 1 | 124 | 107-128 |
| Synpo | 4 | 120.00, 120.00, 122.00, 140.00 | 559-580, 939-960, 1355-1372, 1846-1870 |
| Taf1 | 2 | 152.00, 130.00 | 197-222, 1920-1952 |
| TagIn2 | 2 | 127.00, 120.00 | 53-79, 454-478 |
| Tead1 | 6 | 143.00, 132.00, 124.00, 122.00, 131.00, | 37-68, 2556-2577, 3260-3287, 3782-3806, 5527- |
| | | 143.00 | 5546, 6395-6416 |
| Tecpr1 | 1 | 127 | 409-432 |
| Tgfbr2 | 2 | 126.00, 135.00 | 1292-1312, 2514-2535 |
| Thbs1 | 2 | 132.00, 120.00 | 887-908, 1348-1369 |
| Tmem175 | 2 | 151.00, 147.00 | 635-657, 925-951 |
| Tmem20 | 5 | 139.00, 145.00, 122.00, 151.00, 121.00 | 852-876, 914-936, 1358-1383, 1528-1556, 1874- |
| | | | 1894 |
| Tmem41b | 3 | 130.00, 158.00, 126.00 | 19-41, 2054-2075, 2480-2524 |
| Tmem43 | 3 | 120.00, 138.00, 154.00 | 48-74, 494-519, 871-896 |
| Tmem50b | 3 | 156.00, 135.00, 144.00 | 558-583, 872-895, 1257-1279 |
| Tmprss2 | 1 | 128 | 1215-1243 |
| Tnfrsf1b | 3 | 133.00, 137.00, 139.00 | 772-795, 1691-1712, 2242-2276 |
| Tnrc6c | 2 | 146.00, 127.00 | 602-626, 2266-2293 |
| Tspyl1 | 3 | 154.00, 123.00, 120.00 | 457-489, 502-523, 641-662 |
| Ttc39a | 2 | 127.00, 129.00 | 77-97, 412-439 |
| Uba6 | 2 | 140.00, 126.00 | 1-25, 977-999 |
| Ucp2 | 3 | 121.00, 148.00, 138.00 | 514-553, 1996-2018, 2793-2824 |
| Usp22 | 3 | 135.00, 151.00, 138.00 | 100-118, 1761-1780, 1959-1985 |
| Vamp3 | 2 | 149.00, 124.00 | 146-178, 598-621 |
| Vcam1 | 1 | 127 | 288-318 |
| Vps4a | 3 | 162.00, 123.00, 127.00 | 205-232, 347-369, 415-435 |
| Wars | 2 | 162.00, 122.00 | 36-63, 788-813 |
| Wnk2 | 2 | 127.00, 120.00 | 11-33, 54-75 |
| Wwtr1 | 4 | 162.00, 132.00, 134.00, 144.00 | 104-136, 2133-2152, 2693-2724, 3098-3119 |
| Zbtb4 | 7 | 145.00, 135.00, 146.00, 124.00, 120.00, | 93-111, 433-453, 848-873, 1027-1056, 1668-1689, |
| | | 123.00, 123.00 | 3044-3064, 4490-4514 |
| Zbtb45 | 2 | 125.00, 120.00 | 5-28, 43-64 |
| Zdhhc24 | 2 | 132.00, 162.00 | 954-979, 1249-1268 |
| Zfp106 | 2 | 122.00, 147.00 | 620-647, 663-685 |
| Zfp426 | 4 | 135.00, 120.00, 164.00, 140.00 | 145-161, 408-435, 473-497, 577-599 |
| Zfp704 | 6 | 124.00, 150.00, 131.00, 166.00, 124.00, | 1829-1850, 7600-7623, 8345-8361, 9247-9273, |
| | | 124.00 | 9410-9431, 10699-10721 |

^{¶}positions of mir-122-binding sites in the 3′UTR (the nucleotide after the stop codon is numbered as #1)

We experimentally verified eight novel mir-122 target genes, AlpI, Cs, Ctgf, Igf2, Jun, Klf6, Prom1 and Sox4, that might be relevant to the control of liver diseases (FIG. 10c, Supplementary Table 6).

SUPPLEMENTARY TABLE 6
Experimentally verified miR-122 target genes.

| | | | |
|---|---|---|---|
| Target genes | Species | Validation methods | Refs. |

Functional miRNA-target interactions (Positive samples)

| | | | |
|---|---|---|---|
| AACS | H. sapiens | Reporter assay; qRT-PCR | Tsai, W. C. et al., Hepatology 49, 1571-82 (2009) |
| ADAM10 | H. sapiens | Reporter assay | Bai, S. et al., J Biol Chem 284, 32015-27 (2009) |
| ADAM17 | H. sapiens | Reporter assay; qRT-PCR | Tsai, W. C. et al., Hepatology 49, 1571-82 (2009) |
| AKT3 | H. sapiens | Reporter assay; qRT-PCR | Id. |
| ALDOA | H. sapiens; | Reporter assay; Western blot; | Esau, C. et al., Cell Metab 3, 87-98 (2006); |
| | M. musculus | qRT-PCR | Krutzfeldt, J. et al., Nature 438, 685-9 (2005); |
| | | | Tsai, W. C. et al., Hepatology 49, 1571-82 (2009); |
| | | | Elmen, J. et al., Nucleic Acids Res 36, 1153-62 (2008); |
| | | | Fabani, M. M. et al., RNA 14, 336-46 (2008); |
| | | | Akinc, A. et al., Nat Biotechnol 26, 561-9 (2008) |
| Alpl | M. musculus | Reporter assay | This study |
| ANK2 | H. sapiens | Reporter assay; qRT-PCR | Tsai, W. C. et al., Hepatology 49, 1571-82 (2009) |
| ANXA11 | H. sapiens | Reporter assay; qRT-PCR | Id. |
| AP3M2 | H. sapiens | Reporter assay; qRT-PCR | Id. |
| Apob | M. musculus | Western blot | El Ouaamari, A. et al., Diabetes 57, 2708-17 (2008) |
| ATP1A2 | H. sapiens | Reporter assay; qRT-PCR | Tsai, W. C. et al., Hepatology 49, 1571-82 (2009) |
| Bach1 | M. musculus | qRT-PCR | Shan, Y. et al, Gastroenterology 133, 1166-74 (2007) |
| Bckdk | M. musculus | Reporter assay; Western blot; | Elmen, J. et al., Nucleic Acids Res 36, 1153-62 (2008) |
| | | qRT-PCR | |
| BCL2L2 | H. sapiens | Reporter assay; Western blot; | Lin, C. J., et al., Biochem Biophys Res Commun 375, |
| | | qRT-PCR | 315-20 (2008); |
| | | | Xu, H. et al., Hepatology 52, 1431-42 (2010) |
| CCNG1 | H. sapiens; | Reporter assay; qRT-PCR | El Ouaamari, A. et al., Diabetes 57, 2708-17 (2008); |
| | M. musculus | | Lin, C. J., et al., Biochem Biophys Res Commun 375, |
| | | | 315-20 (2008); |
| | | | Gramantieri, L. et al., Cancer Res 67, 6092-9 (2007); |
| | | | Xu, H. et al. , Hepatology 52, 1431-42 (2010) |
| Ccrn4L | M. musculus | Reporter assay; Western blot; | Gramantieri, L. et al., Cancer Res 67, 6092-9 (2007); |
| | | qRT-PCR | |
| Cd320 | M. musculus | Reporter assay; Western blot; | Elmen, J. et al., Nucleic Acids Res 36, 1153-62 (2008) |
| | | qRT-PCR | |
| CLIC4 | H. sapiens | Reporter assay | Xu, H. et al., Hepatology 52, 1431-42 (2010) |
| Cs | M. musculus | Reporter assay | This study |
| CTCF | H. sapiens | Reporter assay | Xu, H. et al., Hepatology 52, 1431-42 (2010) |
| Ctgf | M. musculus | Reporter assay | This study |
| CUX1 | H. sapiens | Reporter assay | Xu, H. et al., Hepatology 52, 1431-42 (2010) |
| CYP7A1 | H. sapiens | Reporter assay; qRT-PCR | Song, K. H. et al., J Lipid Res 51, 2223-33 (2010) |
| Ddc | M. musculus | Reporter assay | Gatfield, D. et al., Genes Dev 23, 1313-26 (2009) |
| DSTYK | H. sapiens | Reporter assay; qRT-PCR | Tsai, W. C. et al., Hepatology 49, 1571-82 (2009) |
| DUSP2 | H. sapiens | Reporter assay; qRT-PCR | Id. |
| DUSP2 | H. sapiens | Reporter assay; qRT-PCR | Id. |
| EGLN3 | H. sapiens | Reporter assay; qRT-PCR | Id. |
| ENTPD4 | H. sapiens | Reporter assay; qRT-PCR | Id. |
| FAM117B | H. sapiens | Reporter assay; qRT-PCR | Id. |
| FOXJ3 | H. sapiens | Reporter assay; qRT-PCR | Id. |
| FOXP1 | H. sapiens | Reporter assay; qRT-PCR | Id. |
| FUNDC2 | H. sapiens | Reporter assay; qRT-PCR | Id. |
| G6PC3 | H. sapiens | Reporter assay; qRT-PCR | Id. |
| GALNT10 | H. sapiens | Reporter assay; qRT-PCR | Id. |
| Gpx7 | M. musculus | Reporter assay | Fabani, M. M. et al., RNA 14, 336-46 (2008) |
| GTF2B | H. sapiens | qRT-PCR | Fabani, M. M. et al., RNA 14, 336-46 (2008) |
| GYS1 | H. sapiens; | Western blot; qRT-PCR | El Ouaamari, A. et al., Diabetes 57, 2708-17 (2008); |
| | M. musculus | | Fabani, M. M. et al., RNA 14, 336-46 (2008) |
| Hfe2 | M. musculus | Reporter assay | Krutzfeldt, J. et al., Nature 438, 685-9 (2005); |
| | | | Akinc, A. et al., Nat Biotechnol 26, 561-9 (2008) |
| Hist1H1C | M. musculus | Reporter assay | Gatfield, D. et al., Genes Dev 23, 1313-26 (2009) |
| IGF1R | H. sapiens | Reporter assay | Bai, S. et al., J Biol Chem 284, 32015-27 (2009) |
| Igf2 | M. musculus | Reporter assay | This study |
| Jun | M. musculus | Reporter assay | This study |
| Klf6 | M. musculus | Reporter assay | This study |
| LAMC1 | H. sapiens | Reporter assay | Xu, H. et al., Hepatology 52, 1431-42 (2010) |
| Lass6 | M. musculus | Reporter assay | Akinc, A. et al., Nat Biotechnol 26, 561-9 (2008) |
| MAP3K12 | H. sapiens | Reporter assay | Xu, H. et al., Hepatology 52, 1431-42 (2010) |
| MAP3K3 | H. sapiens | Reporter assay | Id. |
| MAPK11 | H. sapiens | Reporter assay; qRT-PCR | Tsai, W. C. et al., Hepatology 49, 1571-82 (2009) |
| MARK1 | H. sapiens | Reporter assay | Xu, H. et al., Hepatology 52, 1431-42 (2010) |
| MECP2 | H. sapiens | Reporter assay; qRT-PCR | Tsai, W. C. et al., Hepatology 49, 1571-82 (2009) |
| NCAM1 | H. sapiens | Reporter assay; qRT-PCR | Id. |
| Ndrg3 | M. musculus | Reporter assay; Western blot; | Krutzfeldt, J. et al., Nature 438, 685-9 (2005); |
| | | qRT-PCR | Elmen, J. et al., Nucleic Acids Res 36, 1153-62 (2008) |
| NFATC2IP | H. sapiens | Reporter assay; qRT-PCR | Tsai, W. C. et al., Hepatology 49, 1571-82 (2009) |
| NUMBL | H. sapiens | Reporter assay; qRT-PCR | Id. |
| P4Ha1 | M. musculus | qRT-PCR | El Ouaamari, A. et al., Diabetes 57, 2708-17 (2008) |
| Ppard | M. musculus | Reporter assay | Gatfield, D. et al., Genes Dev 23, 1313-26 (2009) |
| Prom1 | M. musculus | Reporter assay | This study |
| RAB11FIP1 | H. sapiens | Reporter assay; qRT-PCR | Tsai, W. C. et al., Hepatology 49, 1571-82 (2009) |
| RAB6B | H. sapiens | Reporter assay; qRT-PCR | Id. |
| RAD21 | H. sapiens | Reporter assay | Xu, H. et al., Hepatology 52, 1431-42 (2010) |
| Rcan1 | M. musculus | Reporter assay | Gatfield, D. et al., Genes Dev 23, 1313-26 (2009) |
| Rell1 | M. musculus | Reporter assay | Gatfield, D. et al., Genes Dev 23, 1313-26 (2009) |
| RHOA | H. sapiens | Reporter assay | Coulouarn, C. et al., Oncogene 28, 3526-36 (2009) |
| Sbk1 | M. musculus | Reporter assay | Gatfield, D. et al., Genes Dev 23, 1313-26 (2009) |
| Slc35A4 | M. musculus | Reporter assay | Krutzfeldt, J. et al., Nature 438, 685-9 (2005); |
| | | | Akinc, A. et al., Nat Biotechnol 26, 561-9 (2008) |
| SLC7A1 | H. sapiens; | Reporter assay; Western blot; | El Ouaamari, A. et al., Diabetes 57, 2708-17 (2008) |
| | M. musculus | qRT-PCR | Tsai, W. C. et al., Hepatology 49, 1571-82 (2009); |
| | | | Coulouarn, C. et al., Oncogene 28, 3526-36 (2009) |
| | | | Fabani, M. M. et al., RNA 14, 336-46 (2008); |
| | | | Akinc, A. et al., Nat Biotechnol 26, 561-9 (2008) |
| SLC7A11 | H. sapiens | Reporter assay; qRT-PCR | Tsai, W. C. et al., Hepatology 49, 1571-82 (2009) |
| Smarcd1 | M. musculus | Reporter assay | Gatfield, D. et al., Genes Dev 23, 1313-26 (2009) |
| Sox4 | M. musculus | Reporter assay | This study |
| SRF | H. sapiens | Reporter assay | Bai, S. et al., J Biol Chem 284, 32015-27 (2009) |
| TBX19 | H. sapiens | Reporter assay; qRT-PCR | Tsai, W. C. et al., Hepatology 49, 1571-82 (2009) |
| Tgfbr1 | M. musculus | Reporter assay | Gatfield, D. et al., Genes Dev 23, 1313-26 (2009) |
| Tmed3 | M. musculus | Reporter assay | Krutzfeldt, J. et al., Nature 438, 685-9 (2005); |
| | | | Akinc, A. et al., Nat Biotechnol 26, 561-9 (2008) |
| Tmem50B | M. musculus | Reporter assay | Akinc, A. et al., Nat Biotechnol 26, 561-9 (2008) |
| TPD52L2 | H. sapiens | Reporter assay; qRT-PCR | Tsai, W. C. et al., Hepatology 49, 1571-82 (2009) |
| TRIB1 | H. sapiens | Reporter assay; qRT-PCR | Id. |
| UBAP2 | H. sapiens | Reporter assay; qRT-PCR | Id. |
| VAV3 | H. sapiens | Reporter assay | Xu, H. et al., Hepatology 52, 1431-42 (2010) |
| XPO6 | H. sapiens | Reporter assay; qRT-PCR | Tsai, W. C. et al., Hepatology 49, 1571-82 (2009) |

Non-functional miRNA-target interactions (Negative samples)

| | | | |
|---|---|---|---|
| MSN | H. sapiens | Reporter assay | Xu, H. et al., Hepatology 52, 1431-42 (2010) |
| B2m | M. musculus | Reporter assay | This study |
| Afp | M. musculus | Reporter assay | This study |
| Ccl2 | M. musculus | Reporter assay | This study |
| Csf3r | M. musculus | Reporter assay | This study |
| Cxcl13 | M. musculus | Reporter assay | This study |
| Cyp2b13 | M. musculus | Reporter assay | This study |
| Dbp | M. musculus | Reporter assay | This study |
| Il1b | M. musculus | Reporter assay | This study |
| Per1 | M. musculus | Reporter assay | This study |
| Ccnd1 | M. musculus | Reporter assay | Gatfield, D. et al., Genes Dev 23, 1313-26 (2009) |
| Irf6 | M. musculus | Reporter assay | Id. |
| Socs2 | M. musculus | Reporter assay | Id. |
| Rbl2 | M. musculus | Reporter assay | Id. |
| Camk2b | M. musculus | Reporter assay | Id. |
| Tmem20 | M. musculus | Reporter assay | Id. |
| Gapdh | M. musculus | Reporter assay | Elmen, J. et al., Nucleic Acids Res 36, 1153-62 (2008) |

H. sapiens, Homo sapiens; M. musculus, Mus musculus

In the absence of cholestasis, the elevated expression of AlpI in mir-122 deficiency seems to offer a reasonable explanation for the higher serum ALP levels in mutant mice. KLF6 is a pro-fibrogenic transcription factor known to transactivate the gene expression of TGFβ1, TGFβR1, TGFβR2 and β1 collagen.

We further performed binding site mutation analysis and confirmed the predicted sites in the 3′UTR of the Klf6 transcript (FIGS. 10e-10g). To elucidate the pro-fibrogenic potential of Klf6 and Ctgf in mir-122 deficiency, we downregulated their elevated expressions using the shRNA approach.

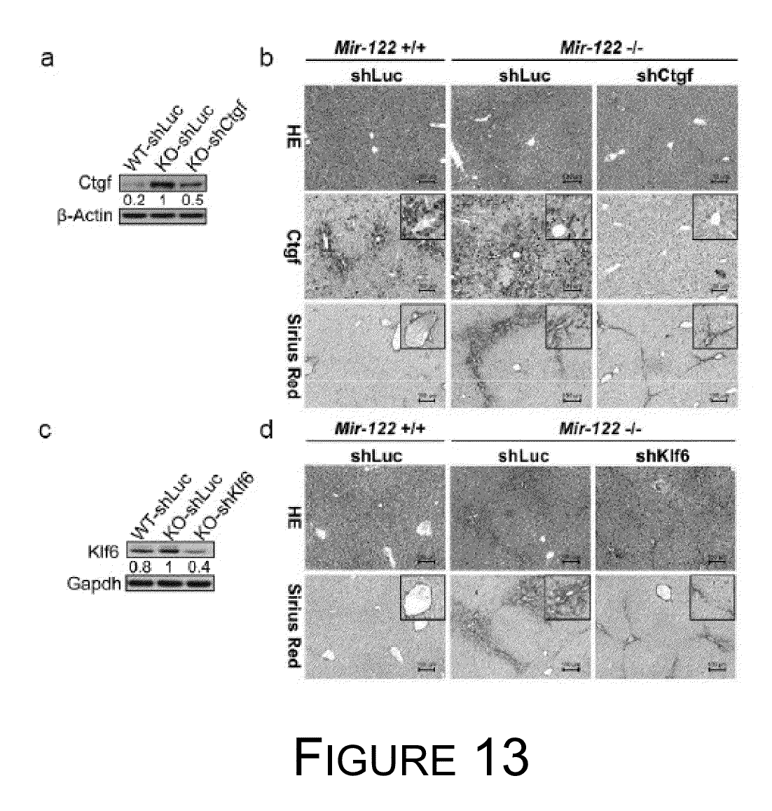

In vivo suppression of either Klf6 or Ctgf led to a decrease in the collagen deposition (FIG. 13). These preliminary results provided important initial evidence to elucidate the mechanism behind mir-122 and its prevention of liver fibrosis. Moreover, the identification of Igf2, Prom1, Jun and Sox4 as targets of mir-122 corroborates the notion that mir-122 deficiency facilitates EMT in livers.

### Methods of the Present Disclosure

### Serological Analysis

Serum biochemical studies including total cholesterol, triglyceride, alanine aminotransferase (ALT) and alkaline phosphatase (ALP) were performed monthly. Serum was collected and analyzed using a DRI-CHEM3500S (FUJIFILM).

### Histology and Immunohistochemistry

Resected liver tissue was processed for either paraffin sections or cryosections. Oil Red O staining was performed on frozen sections fixed with formalin. The paraffin sections were processed for hematoxylin and eosin staining, periodic acid-Schiff (PAS) staining and immunohistochemical staining, the latter using antibodies against F4/80 (Abcam), Desmin (Milipore), Pcna (Abcam), Ecadherin (Cell Signaling), and Vimentin (Abcam).

### RNA Isolation, High-Density Oligonucleotide Microarray Analysis and Expression Validation

The microarray hybridizations were performed using total RNA prepared from the liver samples of three wild-type mice and four mir-122^{-/-} mice at an age of 2-months. Super RNApure (Geneisis Biotech Inc, Taiwan) was used to extract total RNA from the frozen liver samples. GeneChip U133 plus2 Affymetrix oligonucleotide Gene Chips (Affymetrix, Santa Clara, Calif.) were analyzed at Microarray & Gene Expression Analysis Core Facility (VGH-YM Genome Center, National Yang-Ming University) according to the Affymetrix protocols. The arrays were scanned using an Affymetrix GeneChip scanner 3000. The resulting image data was captured and converted to digital output using GeneChip Operating Software v.1.4.0.036. The absolute results (*.chp) from various experiments (probe arrays of the same type) that were scaled to the same target signal using the All Probe Sets scaling option (scaling factor, 500) so that direct comparison was possible (Parameter: Alpha1=0.05, Alpha2=0.065, Tau=0.015). Gene expression was quantified by robust multi-array analysis (RMA) using the Genomic Suite software from Partek. All the data files are presented in compliance with MIAMI guidelines and can be accessed online at the Gene Expression Omnibus (series accession number GSE27713). Microarray dataset was ranked using the expression ratio between mir-122^{-/-} and wild type, and then analyzed using the Gene Set Enrichment Analysis (GSEA, Version 3.2) from Broad Institute. Probe sets were collapsed to genes using median values and Signal2Noise method for GSEA (Gene set enrichment analysis). The differentially expressed genes are listed in Supplementary Table 3.

Expression analysis of mir-122 was done by TaqMan® MicroRNA Assay (Applied Biosystems). Gene expression was detected by quantitative real-time polymerase chain reaction (qRT-PCR) using the SYBR Green I protocol (Bio-Rad). All values were normalized against GAPDH mRNA. The primer sequences are listed in Supplementary Table 7.

SUPPLEMENTARY TABLE 7
Nucleotide sequences of the PCR primers for
the qRT-PCR assays

| | | | SEQ | | | | SEQ |
|---|---|---|---|---|---|---|---|
| | | | ID | | | | ID |
| Gene | | Sequence | NO: | Gene | | Sequence | NO: |
| Acaca | F | GGATTCCACGAAAAGAGC | 1 | Mlxipl | F | GCGCTTTGACCAGATG | 33 |
| | R | GCTGTAGCAAAAGTGGAG | 2 | | R | GGAAGTGCTGAGTTGGC | 34 |
| Acly | F | ATGCGAGTGCAGATCC | 3 | Mttp | F | AGGCAATTCGAGACAAAG | 35 |
| | R | AAGGTAGTGCCCAATG | 4 | | R | ACGTCAAAGCATATCGTTC | 36 |
| Afp | F | TCCAGAAGGAAGAGTGGAC | 5 | Nr1h2 | F | GTGGTGTCTTCTTGAAGATGG | 37 |
| | R | AGACTAGGAGAAGAGAAATAGTT | 6 | | R | CACTCTTGGAAGACTCAATGG | 38 |
| B2m | F | GACCCTAGTCTTTCTGGTGC | 7 | Nr1h3 | F | TGTCCACGAGTGACTGTTTC | 39 |
| | R | TTGCTATTTCTTTCTGCGTGC | 8 | | R | CTGTTGACTCTCCCTTAATGC | 40 |
| Cd90 | F | CCCCAGACAGCGAGAGTCTT | 9 | Prom1 | F | GCTCGTTTTGGAGCTAC | 41 |
| | R | GCCCCTGAGATTAGGAGGTCTT | 10 | | R | ATTCTTACAAACCAGAGACTG | 42 |
| Cdh1 | F | GGAAATGCACCCCTCCAAT | 11 | Pklr | F | AGGAGTCTTCCCCTTG | 43 |
| | R | AATCGGCCAGCATTTTCTGT | 12 | | R | GCGTTTCAGGATATGGTC | 44 |
| Cpt1a | F | ACTGTAAGTCAAAGCCG | 13 | Ppara | F | GCTAATAGGATTCAGACAGTGAC | 45 |
| | R | CAGTGAAAGCCCACTC | 14 | | R | GATTTAAGAGAGTGCACATAGCC | 46 |
| Cpt2 | F | ATGCTGTTCACGATGAC | 15 | Pparg | F | GTCCATGAGATCATCTACACG | 47 |
| | R | CTCATTACCTTCAGTTGGG | 16 | | R | ACTGTCATCTAATTCCAGTGC | 48 |
| Epcam | F | CAGCTGGACACCGGCATT | 17 | Scd1 | F | TAATTGAACACGCGCTC | 49 |
| | R | TGGACCTGCACCTATAAGACGTT | 18 | | R | ACACCAGGACCTCAATG | 50 |
| Fasn | F | CCAAACTGAGCCTTTTCTACC | 19 | Slc27a5 | F | CTTGTTGCGAATGTACGAC | 51 |
| | R | AGAAACTTTCCCAGAAATCTTCC | 20 | | R | GATACGGATGAAATGAGGTG | 52 |
| Foxa1 | F | TGGTCATGTCATGCTGAG | 21 | Slco1a1 | F | TTCAACTGGCCTGTGC | 53 |
| | R | CACTGGATGAGCCAAG | 22 | | R | GTGCGTCACCGTAGATG | 54 |
| Foxa2 | F | GCCTATTATGAACTCATCCTAAG | 23 | Slco1a4 | F | CCTGTCACACAGTTGG | 55 |
| | R | GAATGACAGATCACTGTGG | 24 | | R | CCACCGAGATACAGCC | 56 |
| Gsta2 | F | AAACCGTTACTTGCCTG | 25 | Sox4 | F | CTCGCCTTGGTGATTTC | 57 |
| | R | TCCAAGGGAGGCTTTC | 26 | | R | CCTAAGCTCAACACAAATGC | 58 |
| Igf2 | F | CTTGTCTCTTCCCTACTG | 27 | Srebf1 | F | CGCACCGTAGAGAAGC | 59 |
| | R | AGGTTTGCGAGCGTTA | 28 | | R | CTAGAGGTCGGCATGG | 60 |
| Klf6 | F | AGATCCTTCTATTTTG | 29 | Src | F | AGGAACTAACGAGAACTGT | 61 |
| | R | CTAGACAGGTACTCAA | 30 | | R | ACCACCACTTCTACCC | 62 |
| Ldlr | F | CAACACTAACACGGAG | 31 | Vim | F | TCAAGTGCCTTTACTGCAGTTTTT | 63 |
| | R | AGTACCGAATGTCACGAG | 32 | | R | TGCTGAGCTTCTTTCTATTCCAAA | 64 |

F, forward primer; R, reverse primer

### Lipoprotein Electrophoresis

Blood for mouse serum lipoprotein analysis was obtained following two consecutive overnights (16 h) of fasting. Serum lipoproteins were analyzed on the Hydragel K20 electrophoresis System (Sebia, France) according to the manufacture's methodology.

Extraction of Total Lipids from Liver

Mice were fasted for two consecutive overnights (16 h) before liver tissue sampling. A 0.2-0.5 g portion of the liver was frozen in lipid nitrogen and ground into a powder in a mortar. A 4 ml mixture of chloroform and methanol was added to create a suspension to allow the extraction of lipids. The procedure was repeated twice. A total of 12 ml of extraction solution was used. The mixtures containing the extracted lipids were pooled into a 20-ml saponification tube. After adding 3 ml distilled water into the mortar to resuspend the tissue material, the suspension was added to the extracts. The pooled suspension was then extensively vortexed (30 sec×4) followed by centrifuging at 2,500 rpm for 30 min. A 4-ml portion of the upper layer and a 5-ml portion of the bottom layer were separately collected into 20 ml counting vials. The organic (bottom) layer was dried under a stream of N₂ gas. The upper aqueous layer was concentrated on a centrifugal concentrator. The two residues were then stored at −80° C. before NMR measurement.

### ¹H-NMR Measurement

The lipid residues were re-suspended in 400 μl deuterated chloroform (CDCl₃). The solution was transferred to a 5 mm NMR tube. NMR measurements were carried out on a 400 MHZ FT-NMR spectrometer (Bruker) with a BDI probehead. The pulse sequence and data acquisition for the NMR measurements were similar to those reported by Beckonert (Beckonert, O. et al., Nat. Protoc 2, 2692-703 (2007)). A reference sample containing 2 mg cholesterol in CDCl₃ and under the same NMR conditions was used for comparison and quantification (signal intensity of H-18, chemical shift 0.65 ppm).

Identifying miR-122 Targets in the Up-Regulated Genes of mir122^{-/-} Livers

Three computational tools, namely miRanda, TargetScanS and RNAhybrid, which had successfully integrated by us in miRNAMap previously (Hsu, S. D. et al., Nucleic Acids Res 36, D165-9 (2008)) were used in this study. In order to achieve higher prediction accuracy, we also integrated another tool, PITA. The integrated tools were then used to identify the miR-122 target sites located within the accessible regions of 3′-UTR of up-regulated genes in the mir122^{-/-} mouse liver. Up-regulated orthologous genes with target sites in both the mouse and human genomes were pinpointed.

The predictive parameters of each miRNA target prediction tool were optimized to yield a better set of miRNA target candidates (See Performance Evaluation). Furthermore, we recalculated the miRNA/target duplex score using the following single-position base-pairing values. A score of +5 was assigned for G:C and A:U pairs, +2 for G:U wobble pairs, and −3 for mismatch pairs, and the gap-open and gap-elongation parameters were set to −8.0 and −2.0, respectively. The match value s(i) is multiplied by a position specific weight w(i). The position specific weights emphasize the importance of the ‘seed region’ generally defined as the position 2-8 of the miRNA 5′-end. Thus the total score S for a particular alignment is

$$S = {\sum }_{i = 1}^{n} W ( i ) X s ( i )$$

A higher score indicates a more stable miRNA/target duplex. In the end 252 up-regulated orthologous genes, which were identified by at least three target prediction tools, were selected for experimental validation and further analysis (Supplementary Table 5).

### Performance Evaluation

In order to evaluate the performance of miRNA target prediction tools and our proposed method, we collected 80 experimentally validated miR-122 target genes and 18 miRNA non-target genes (Supplementary Table 6). This dataset is based on our validated miR-122 targets and was complemented by additional validated targets curated from miRTarBase. Before the comparing prediction accuracy of the target prediction tools and our proposed method, the parameters used by miRanda and RNAhybrid were optimized. The miRanda score was adjusted from 100 to 180 using step=5 and MFE was set from −10 kcal/mol to −30 kcal/mol with step=−2 kcal/mol. Furthermore, RNAhybrid MFE was adjusted from −10 kcal/mol to −30 kcal/mol with step=−2 kcal/mol. The predictive parameters of TargetScanS and PITA were set at their default values. The optimal parameters of each target prediction tool were determined by the maximizing the performance (PERF) using the following formula:

$$PERF - Sensitivity ( SENS ) \times Specificity ( SPEC ) ;$$

$$SENS - \frac{TP}{TP + FN} ;$$

$$SPEC - \frac{TN}{FP + TN}$$

In the equation, TN represents true negative, TP true positive, FN false negative and FP false positive. The MFE threshold of the miRNA and target duplex was −7 kcal/mol and the miRanda score cutoff was specified as 120. The MFE threshold of the miRNA and target duplex in RNAhybrid was set to −23 kcal/mol. The performances of the individual prediction tools and our combinatory method are displayed in Supplementary Table 8. We found that miRanda has the highest sensitivity, while TargetScanS has the highest specificity. It can be seen that our combinatory method is the best approach to the identification of miR-122 targets.

SUPPLEMENTARY TABLE 8
Performance comparisons of miRNA target prediction tools.

| | | Sensitivity | Specificity | Accuracy | PERF* |
|---|---|---|---|---|---|
| Performance of each tool | miRanda | 91.3% | 38.9% | 81.6% | 0.355 |
| | TargetScanS | 58.8% | 77.8% | 62.2% | 0.457 |
| | RNAhybrid | 68.8% | 66.7% | 68.4% | 0.459 |
| | PITA | 85.0% | 44.4% | 77.6% | 0.377 |
| Performance of integrated tools | At least 3 tools | 77.5% | 72.2% | 76.5% | 0.560 |
| ( This study ) | | | | | |

*PERF (Performance) = Sensitivity × Specificity

3′UTR Reporter Assay

The 3′UTR fragments of the candidate target genes were subcloned into the XhoI and NotI site downstream of the luciferase gene in the vector psi-CHECK2 (Promega, Madison Wis.). The negative controls were lenti-122M and lenti-GFP9. HEK-293T cells were infected with lenti-GFP and lenti-122 or lenti-122M for 24 h. Cells were then seeded into 24-well plate and co-transfected with 0.5 μg of the respective psi-CHECK2-3′UTR construct using jetPEI (Polyplus-Transfection, France). After 48 h, luciferase activity was measured using the Dual-Luciferase Reporter Assay System Kit (Promega). The effect of miR-122 was expressed relative to the average value from cells infected with lenti-GFP virus. Three mutants of the miR-122 binding sites in the 3′ UTR of Klf6 were included in this study, Klf6-mu1, Klf6-mu2, and Klf6-mu1+mu2. The nucleotide sequences of all of the PCR cloning primers (Supplementary Table 9) and mutagenesis primers (Supplementary Table 10) are listed.

```
SUPPLEMENTARY TABLE 9
  Nucleotide sequences
of the PCR cloning primers for the
  3′UTR reporter constructs.
```

| | | | SEQ |
|---|---|---|---|
| Gene | Primers | Sequence | ID NO: |
| Afp | Forward | CTCCGAGTCCAGAAGGAAGAGTGGAC | 65 |
| | Reverse | GCGGCCGCAGACTAGGAGAAGAGAAA | 66 |
| | | TAGTT | |
| Aldoa | Forward | CTCGAGCCAGAGCTGAACTAAGGC | 67 |
| | Reverse | GCGGCCGCCTTAAATAGTTGTTTATTGGC | 68 |
| Alpl | Forward | CTCGAGCAAGCCCGCAATGGAC | 69 |
| | Reverse | GCGGCCGCTCCAAACAGGAGAGCC | 70 |
| B2m | Forward | CTCGAGCTCTGAAGATTCATTTGAACCT | 71 |
| | Reverse | GCGGCCGCGCTAAGCATTGGGCAC | 72 |
| Cs | Forward | CTCGAGGGAATGACCAGCCTCT | 73 |
| | Reverse | GCGGCCGCCATCCTGAAGTCTGCATC | 74 |
| Ctgf | Forward | CTCGAGGCATGTGTCCTCCACT | 75 |
| | Reverse | GCGGCCGCATCGGACCTTACCCTGA | 76 |
| Igf2 | Forward | CTCGAGGACCTCCTCTTGAGCAG | 77 |
| | Reverse | GCGGCCGCTGTGGACAGGTGCTTAGA | 78 |
| Jun | Forward | CTCGAGGCTGAGTGCCCAATATAC | 79 |
| | Reverse | GCGGCCGCAGAGAAAGCTCACC | 80 |
| Klf6 | Forward | CTCGAGCTGGCAAGACACGTTC | 81 |
| | Reverse | GCGGCCGCCTTTCAGTATTACCAACAG | 82 |
| | | ATAGC | |
| Prom1 | Forward | CTCGAGTTTGGAGCTACCTGCG | 83 |
| | Reverse | GCGGCCGCGAACGTAATGCCCATTCT | 84 |
| Sox4 | Forward | CTCGAGTAGAGCTGGCCTGGAAC | 85 |
| | Reverse | GCGGCCGCCTTGACCATGAGGCAAAAT | 86 |

SUPPLEMENTARY TABLE 10
RT-PCR primers used in mutagenesis
reactions.

| Gene | Primers | Sequence | SEQ ID NO: |
|---|---|---|---|
| Klf6-M1 | Forward | CCTTCTATTTTGTAGCGCGCACATGCAAAATGATCTTG | 87 |
| | Reverse | CAAGATCATTTTGCATGTGCGCGCTGCAAAATAGAAGG | 88 |
| Klf6-M2 | Forward | CATACACACACGCGCGCGCAGGCTGTATTTATTATG | 89 |
| | Reverse | CATAATAAATACAGCCTGCGCGCGCGTGTGTGTATG | 90 |

### Western Blotting

Immunoblotting was performed as described previously (Naugler, W. E. et al., Science 317, 121-4 (2007)). Protein lysate (30 μg) was electrophoresed on 10% SDS polyacrylamide gels and transferred onto PVDF membranes (Millipore). The membranes were incubated with primary antibodies overnight at 4° C. and then with horseradish peroxidase-conjugated secondary antibody (Perkin Elmer Life Sciences). Primary antibodies against Apob 100 (Novus), Apob-48 (Novus), Apoe (Abcam), Mttp (Abcam), Klf6 (Santa Cruz Biotech), Fasn, desmin, Afp, Pten, phosphor-Akt, Akt, phosphor-c-Raf, c-Raf, phosphor-Mek1/2, Mek1/2, phosphor-Erk, Erk, Pcna, Bax, Xiap, Phosphor-Gys2, Gys2 (Cell Signaling Technology), E-cadherin (Cell Signaling), and Vimentin (Abcam) were used. Signals were detected by an enhanced chemiluminescence kit (PerkinElmer, Waltham, Mass.). The relative level of protein expression was normalized against Gapdh.

### Hydrodynamic Injection

A partial human pri-miR-122 gene was subcloned into the vector pcDNA3.1(B) (Invitrogen, Carlsbad, Calif.) and designated pcDNA-miR-122. Plasmid DNA was injected by the hydrodynamic technique as previously described (Yang, P. L. et al., Proc Natl Acad Sci USA 99, 13825-30 (2002)). Briefly, 20 μg of endotoxin-free plasmid DNA was dissolved in 2 ml of sterile pharmaceutical grade saline at room temperature and injected into the mouse tail vein with a 26.5 gauge needle in 6 seconds. All the mice received two injections, one on day 1 and one on day 15. The wild type mice were injected with the pcDNA3.1(B) HA vector DNA only, while the mir122^{-/-} mice were injected with either the pcDNA3.1(B) HA vector DNA or HA-miR-122 DNA. Each group included at least three mice of 3 month old. Serum biochemical studies were carried out at day 5 and day 14. The mice were sacrificed after one month for histological examination and gene expression analysis.

### Statistical Analyses

All data are expressed as means±SD and compared between groups using the Student's t test. A p value<0.05 was considered statistically significant. *p<0.05; **p<0.01; ***p<0.001.

### Implementation and Additional Notes

All references, including publications, patent applications and patents, cited herein are hereby incorporated by reference to the same extent as if each reference was individually and specifically indicated to be incorporated by reference and was set forth in its entirety herein.

Any combination of the above-described elements in all possible variations thereof is encompassed by the application unless otherwise indicated herein or otherwise clearly contradicted by context.

The terms “a” and “an” and “the” and similar referents as used in the context of describing the application are to be construed to cover both the singular and the plural, unless otherwise indicated herein or clearly contradicted by context.

The use of any and all examples, or exemplary language (e.g., “such as”) provided herein, is intended merely to better illuminate the application and does not pose a limitation on the scope of the application unless otherwise indicated. No language in the specification should be construed as indicating any element is essential to the practice of the application unless as much is explicitly stated.

The description herein of any aspect or embodiment of the application using terms such as “comprising,” “having,” “including” or “containing” with reference to an element or elements is intended to provide support for a similar aspect or embodiment of the application that “consists of,” “consists essentially of,” or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context). That said, the terms “comprising,” “having,” “including” or “containing” in the claims should be construed according to the conventional “open” meaning of those terms in the patent law to include those elements enumerated as well as other elements. Likewise, the terms “consisting of,” “consists of,” “consists essentially of,” or “substantially comprises” should be construed according to the “closed” or “partially closed” meanings ascribed to those terms in the patent law.

This disclosure includes all modifications and equivalents of the subject matter recited in the aspects or embodiments presented herein to the maximum extent permitted by applicable law.

1-35. (canceled)

36. A therapeutic for treating and/or preventing liver associated disorders, the therapeutic comprising a delivery vehicle carrying a mir-122 gene.

37. The therapeutic of claim 36, wherein the mir-122 gene is selected from the group consisting of human mir-122 gene and murine mir-122 gene.

38. The therapeutic of claim 36, wherein the delivery vehicle is a vector, a liposome, a polymer, a pharmaceutically acceptable composition, or a device which facilitates delivery of such delivery vehicle.

39. The therapeutic of claim 38, wherein the vector is selected from the group consisting of adenovirus vectors, retrovirus vectors, adeno-associated virus vectors, herpes simplex virus vectors, SV40 vectors, polyoma virus vectors, papilloma virus vectors, picarnovirus vectors, vaccinia virus vectors, lentiviral vectors, alphaviral vectors, a helper-dependent adenovirus, and a plasmid.

40. The therapeutic of claim 36 for use in treating liver associated disorders.

41. The therapeutic of claims 40, wherein the liver associated disorder is selected from the group consisting of hepatic steatosis, hepatitis, liver fibrosis, hepatocyte proliferation, and hepatocellular carcinoma.

42. The therapeutic of claim 36 for use in preventing liver associated disorders.

43. The therapeutic of claim 42, wherein the liver associated disorder is selected from the group consisting of hepatic steatosis, hepatitis, liver fibrosis, hepatocyte proliferation, and hepatocellular carcinoma.

44. A method of preventing and/or treating a liver associated disorder comprising:
administering to a subject in need thereof a therapeutically effective amount of the mir-122 gene.

45. The method of claim 44, wherein the liver associated disorder is selected from the group consisting of hepatic steatosis, hepatitis, liver fibrosis, hepatocyte proliferation, and hepatocellular carcinoma.

46. The method of claim 44, wherein the administering step comprises administering using a delivery vehicle.

47. The method of claim 46, wherein the delivery vehicle is a vector, a liposome, a polymer, a pharmaceutically acceptable composition, or a device which facilitates delivery of such delivery vehicle.

48. The method of claim 47, wherein the vector is selected from the group consisting of adenovirus vectors, retrovirus vectors, adeno-associated virus vectors, herpes simplex virus vectors, SV40 vectors, polyoma virus vectors, papilla a virus vectors, picarnovirus vectors, vaccinia virus vectors, lentiviral vectors, alphaviral vectors, a helper-dependent adenovirus, and a plasmid.

49. The method of claim 44, wherein the administering step includes administering in a manner selected from the group consisting of intravenous administration, subcutaneous administration, intra-bone marrow administration, intra-arterial administration, intra-cardiac administration, intracerebral administration, intraspinal administration, intra-peritoneal administration, intra-muscular administration, parenteral administration, intrarectal administration, intra-tracheal injection, intra-nasal administration, intradermal administration, epidermal administration, oral administration and combinations thereof.

50. The method of claim 44, wherein the administering step includes administering to the subject in need of therapeutically effective amounts of the mir-122 gene.

51. The method of claim 44, wherein the administering step includes administering the mir-122 gene in combination with another therapeutic.

52. The method of claim 44, wherein the subject in need is a human.

53. A method for detecting the presence or a predisposition to a liver associated disorder in a subject, comprising the steps of:
obtaining a test sample from the subject;
determining the level of mir-122 expression in the test sample;
comparing the mir-122 expression level from the test sample to the expression level present in a control sample known not to have, or not to be predisposed to a liver associated disorder,
wherein an alteration in the level of mir-122 expression in the test sample as compared to the control sample indicates the presence or predisposition to a liver associated disorder.

54. The method of claim 53, wherein the liver associated disorder is selected from the group consisting of hepatic steatosis, hepatitis, liver fibrosis, hepatocyte proliferation, and hepatocellular carcinoma.

55. The method of claim 53, wherein the alteration is a decreased level of mir-122 expression in the test sample as compared to the control sample.

56-62. (canceled)


SEQUENCE LISTING (90 sequences)

SEQ ID NO: 1 - DNA, 18 bp, Artificial
  primer
ggattccacgaaaagagc

SEQ ID NO: 2 - DNA, 18 bp, Artificial
  primer
gctgtagcaaaagtggag

SEQ ID NO: 3 - DNA, 16 bp, Artificial
  primer
atgcgagtgcagatcc

SEQ ID NO: 4 - DNA, 16 bp, Artificial
  primer
aaggtagtgcccaatg

SEQ ID NO: 5 - DNA, 19 bp, Artificial
  primer
tccagaaggaagagtggac

SEQ ID NO: 6 - DNA, 23 bp, Artificial
  primer
agactaggagaagagaaatagtt

SEQ ID NO: 7 - DNA, 20 bp, Artificial
  primer
gaccctagtctttctggtgc

SEQ ID NO: 8 - DNA, 21 bp, Artificial
  primer
ttgctatttctttctgcgtgc

SEQ ID NO: 9 - DNA, 20 bp, Artificial
  primer
ccccagacagcgagagtctt

SEQ ID NO: 10 - DNA, 22 bp, Artificial
  primer
gcccctgagattaggaggtctt

SEQ ID NO: 11 - DNA, 19 bp, Artificial
  primer
ggaaatgcacccctccaat

SEQ ID NO: 12 - DNA, 20 bp, Artificial
  primer
aatcggccagcattttctgt

SEQ ID NO: 13 - DNA, 17 bp, Artificial
  primer
actgtaagtcaaagccg

SEQ ID NO: 14 - DNA, 16 bp, Artificial
  primer
cagtgaaagcccactc

SEQ ID NO: 15 - DNA, 17 bp, Artificial
  primer
atgctgttcacgatgac

SEQ ID NO: 16 - DNA, 19 bp, Artificial
  primer
ctcattaccttcagttggg

SEQ ID NO: 17 - DNA, 18 bp, Artificial
  primer
cagctggacaccggcatt

SEQ ID NO: 18 - DNA, 23 bp, Artificial
  primer
tggacctgcacctataagacgtt

SEQ ID NO: 19 - DNA, 21 bp, Artificial
  primer
ccaaactgagccttttctacc

SEQ ID NO: 20 - DNA, 23 bp, Artificial
  primer
agaaactttcccagaaatcttcc

SEQ ID NO: 21 - DNA, 18 bp, Artificial
  primer
tggtcatgtcatgctgag

SEQ ID NO: 22 - DNA, 16 bp, Artificial
  primer
cactggatgagccaag

SEQ ID NO: 23 - DNA, 23 bp, Artificial
  primer
gcctattatgaactcatcctaag

SEQ ID NO: 24 - DNA, 19 bp, Artificial
  primer
gaatgacagatcactgtgg

SEQ ID NO: 25 - DNA, 17 bp, Artificial
  primer
aaaccgttacttgcctg

SEQ ID NO: 26 - DNA, 16 bp, Artificial
  primer
tccaagggaggctttc

SEQ ID NO: 27 - DNA, 18 bp, Artificial
  primer
cttgtctcttccctactg

SEQ ID NO: 28 - DNA, 16 bp, Artificial
  primer
aggtttgcgagcgtta

SEQ ID NO: 29 - DNA, 16 bp, Artificial
  primer
agatccttctattttg

SEQ ID NO: 30 - DNA, 16 bp, Artificial
  primer
ctagacaggtactcaa

SEQ ID NO: 31 - DNA, 16 bp, Artificial
  primer
caacactaacacggag

SEQ ID NO: 32 - DNA, 18 bp, Artificial
  primer
agtaccgaatgtcacgag

SEQ ID NO: 33 - DNA, 16 bp, Artificial
  primer
gcgctttgaccagatg

SEQ ID NO: 34 - DNA, 17 bp, Artificial
  primer
ggaagtgctgagttggc

SEQ ID NO: 35 - DNA, 18 bp, Artificial
  primer
aggcaattcgagacaaag

SEQ ID NO: 36 - DNA, 19 bp, Artificial if
  primer
acgtcaaagcatatcgttc

SEQ ID NO: 37 - DNA, 21 bp, Artificial
  primer
gtggtgtcttcttgaagatgg

SEQ ID NO: 38 - DNA, 21 bp, Artificial
  primer
cactcttggaagactcaatgg

SEQ ID NO: 39 - DNA, 20 bp, Artificial
  primer
tgtccacgagtgactgtttc

SEQ ID NO: 40 - DNA, 21 bp, Artificial
  primer
ctgttgactctcccttaatgc

SEQ ID NO: 41 - DNA, 17 bp, Artificial
  primer
gctcgttttggagctac

SEQ ID NO: 42 - DNA, 21 bp, Artificial
  primer
attcttacaaaccagagactg

SEQ ID NO: 43 - DNA, 16 bp, Artificial
  primer
aggagtcttccccttg

SEQ ID NO: 44 - DNA, 18 bp, Artificial
  primer
gcgtttcaggatatggtc

SEQ ID NO: 45 - DNA, 23 bp, Artificial
  primer
gctaataggattcagacagtgac

SEQ ID NO: 46 - DNA, 23 bp, Artificial
  primer
gatttaagagagtgcacatagcc

SEQ ID NO: 47 - DNA, 21 bp, Artificial
  primer
gtccatgagatcatctacacg

SEQ ID NO: 48 - DNA, 21 bp, Artificial
  primer
actgtcatctaattccagtgc

SEQ ID NO: 49 - DNA, 17 bp, Artificial
  primer
taattgaacacgcgctc

SEQ ID NO: 50 - DNA, 17 bp, Artificial
  primer
acaccaggacctcaatg

SEQ ID NO: 51 - DNA, 19 bp, Artificial
  primer
cttgttgcgaatgtacgac

SEQ ID NO: 52 - DNA, 20 bp, Artificial
  primer
gatacggatgaaatgaggtg

SEQ ID NO: 53 - DNA, 16 bp, Artificial
  primer
ttcaactggcctgtgc

SEQ ID NO: 54 - DNA, 17 bp, Artificial
  primer
gtgcgtcaccgtagatg

SEQ ID NO: 55 - DNA, 16 bp, Artificial
  primer
cctgtcacacagttgg

SEQ ID NO: 56 - DNA, 16 bp, Artificial
  primer
ccaccgagatacagcc

SEQ ID NO: 57 - DNA, 17 bp, Artificial
  primer
ctcgccttggtgatttc

SEQ ID NO: 58 - DNA, 20 bp, Artificial
  primer
cctaagctcaacacaaatgc

SEQ ID NO: 59 - DNA, 16 bp, Artificial
  primer
cgcaccgtagagaagc

SEQ ID NO: 60 - DNA, 16 bp, Artificial
  primer
ctagaggtcggcatgg

SEQ ID NO: 61 - DNA, 19 bp, Artificial
  primer
aggaactaacgagaactgt

SEQ ID NO: 62 - DNA, 16 bp, Artificial
  primer
accaccacttctaccc

SEQ ID NO: 63 - DNA, 24 bp, Artificial
  primer
tcaagtgcctttactgcagttttt

SEQ ID NO: 64 - DNA, 24 bp, Artificial
  primer
tgctgagcttctttctattccaaa

SEQ ID NO: 65 - DNA, 26 bp, Artificial
  primer
ctccgagtccagaaggaagagtggac

SEQ ID NO: 66 - DNA, 31 bp, Artificial
  primer
gcggccgcagactaggagaagagaaatagtt

SEQ ID NO: 67 - DNA, 24 bp, Artificial
  primer
ctcgagccagagctgaactaaggc

SEQ ID NO: 68 - DNA, 29 bp, Artificial
  primer
gcggccgccttaaatagttgtttattggc

SEQ ID NO: 69 - DNA, 22 bp, Artificial
  primer
ctcgagcaagcccgcaatggac

SEQ ID NO: 70 - DNA, 24 bp, Artificial
  primer
gcggccgctccaaacaggagagcc

SEQ ID NO: 71 - DNA, 28 bp, Artificial
  primer
ctcgagctctgaagattcatttgaacct

SEQ ID NO: 72 - DNA, 24 bp, Artificial
  primer
gcggccgcgctaagcattgggcac

SEQ ID NO: 73 - DNA, 22 bp, Artificial
  primer
ctcgagggaatgaccagcctct

SEQ ID NO: 74 - DNA, 26 bp, Artificial
  primer
gcggccgccatcctgaagtctgcatc

SEQ ID NO: 75 - DNA, 22 bp, Artificial
  primer
ctcgaggcatgtgtcctccact

SEQ ID NO: 76 - DNA, 25 bp, Artificial
  primer
gcggccgcatcggaccttaccctga

SEQ ID NO: 77 - DNA, 23 bp, Artificial
  primer
ctcgaggacctcctcttgagcag

SEQ ID NO: 78 - DNA, 26 bp, Artificial
  primer
gcggccgctgtggacaggtgcttaga

SEQ ID NO: 79 - DNA, 24 bp, Artificial
  primer
ctcgaggctgagtgcccaatatac

SEQ ID NO: 80 - DNA, 22 bp, Artificial
  primer
gcggccgcagagaaagctcacc

SEQ ID NO: 81 - DNA, 22 bp, Artificial
  primer
ctcgagctggcaagacacgttc

SEQ ID NO: 82 - DNA, 32 bp, Artificial
  primer
gcggccgcctttcagtattaccaacagatagc

SEQ ID NO: 83 - DNA, 22 bp, Artificial
  primer
ctcgagtttggagctacctgcg

SEQ ID NO: 84 - DNA, 26 bp, Artificial
  primer
gcggccgcgaacgtaatgcccattct

SEQ ID NO: 85 - DNA, 23 bp, Artificial
  primer
ctcgagtagagctggcctggaac

SEQ ID NO: 86 - DNA, 27 bp, Artificial
  primer
gcggccgccttgaccatgaggcaaaat

SEQ ID NO: 87 - DNA, 38 bp, Artificial
  primer
ccttctattttgtagcgcgcacatgcaaaatgatcttg

SEQ ID NO: 88 - DNA, 38 bp, Artificial
  primer
caagatcattttgcatgtgcgcgctgcaaaatagaagg

SEQ ID NO: 89 - DNA, 36 bp, Artificial
  primer
catacacacacgcgcgcgcaggctgtatttattatg

SEQ ID NO: 90 - DNA, 36 bp, Artificial
  primer
cataataaatacagcctgcgcgcgcgtgtgtgtatg